(19) United States
(12) Patent Application Publication
(10) Pub. No.: US 2012/0059173 A1
(43) Pub. Date: Mar. 8, 2012

(54) DENDRITIC MOLECULES

(75) Inventors: Michael John Monteiro, Brisbane (AU); Michael Raymond Whittaker, Brisbane (AU); Craig Andrew Bell, Brisbane (AU); Carl N. Urbani, Brisbane (AU); Daria Eden Lonsdale, Brisbane (AU)

(73) Assignee: THE UNIVERSITY OF QUEENSLAND, Brisbane (AU)

(21) Appl. No.: 12/597,691

(22) PCT Filed: Apr. 24, 2009
(86) PCT No.: PCT/AU08/00581

§ 371 Date: Nov. 21, 2011

(51) Int. Cl. C07D 403/14

(52) U.S. Cl. 548/255

(57) ABSTRACT

The invention relates to novel dendritic molecules and methods of making them. The dendritic molecules comprise arms, each of which arms is a polymer. The dendritic molecules can be synthesised by way of a reasonably small number of versatile and reliable step-wise reactions, especially click chemistry reactions. Chemical and structural heterogeneity is possible in the dendritic molecule. The invention also provides for surface and interior functionalisation of the molecule.

### FIELD OF THE INVENTION

This invention relates to novel dendrons and dendritic molecules and methods for their preparation.

### BACKGROUND OF THE INVENTION

Any discussion of the prior art throughout the specification should in no way be considered as an admission that such prior art is widely known or forms part of common general knowledge in the field.

Macromolecular architecture traditionally encompasses linear, cross-linked and branched polymers. A common drawback is that the polymers are often polydisperse products of varying molecular weight and structural control is difficult.

In contrast, a dendrimer is a relatively new form of macromolecular architecture, which is highly branched, or tree like, structurally controlled and has narrow polydispersity. The dendrimer is three-dimensional and its size is on the nano scale. The branches and the associated end-groups are built around a multi-functional core molecule.

Dendrimers differ from other hyperbranched polymers in that each of the monomer units in the dendrimer has at least one functional group that allows branching.

Synthesizing monodisperse polymers demands a high level of synthetic control, which is achieved through stepwise reactions, building the dendrimer up one polymer layer, or “generation,” at a time until the terminating generation.

Dendrimers are commonly synthesised by divergent or convergent synthesis. Divergent synthesis starts at the core and builds its way out to the periphery of the dendrimer. In divergent synthesis the dendrimer structure is built up in layers, or generations, from the core, each generation adding another layer to the structure in a radial fashion and increasing the size of the dendrimer. In most known methods, convergent synthesis starts at the periphery (i.e. what will be the surface of the dendrimer) and proceeds inward to the core of the dendrimer. Convergent synthesis involves the production of branches, or dendrons, and then reacting the dendrons with a multi-functional core to produce the dendrimer.

Dendrimers have two major chemical environments, the surface of the dendritic sphere which is the functional groups on the termination generation and the interior which is shielded from exterior environments due to the spherical shape of the dendrimer structure. The functional groups on the terminating generation provide a high degree of surface functionality to the macromolecule. Consequently, dendrimers have myriad potential applications which include areas such as medicine (eg, targeted delivery of pharmaceuticals or diagnostic agents, biomedical coatings, cellular transport), chemistry/engineering (eg, nanoreactors, chemical and biological sensors and detectors, sacrificial porogens, coatings and thin films), consumer goods (eg, inks, toners, dyes, paints, personal products, detergents) and environmental (eg, decontamination agents, filtration agents). Further the size of many dendrimers is in the nano-scale (about 1 to 500 nm). This is advantageous for numerous applications. For example, in the biological field nano-scale dendrimers might be able to cross cell membranes, raise an immune response or avoid rapid clearance by the kidneys and have a long half-life in serum.

As previously mentioned, conventional methods of synthesising dendrimers involve the building up of each generation using small molecules, see for example the PAMAM dendrimers of D. A. Tomalia, H. Baker, J. Dewald, M. Hall, G. Kallos, S. Martin, J. Roeck, J. Ryder and P. Smith, Dendritic Macromolecules: Synthesis of Starburst Dendrimers, Macromolecules, 19 (1986) 2466-2468. Producing dendrimers using small molecules requires a large number of reaction steps resulting in a large number of generations. Due to steric effects, continuing to react dendrimer repeat units can lead to steric overcrowding preventing complete reaction at a specific generation and destroying the molecule's monodispersity. As the number of generations increases, so too does the number of reactive end groups on the last generational layer, which increases the possibility of side reactions occurring. Thus, there is little control of the reaction process. In addition, dendrimers produced using small molecules are only capable of having functionality on the periphery and not in the interior of the molecule. Lastly, the controlled structure results in severe limitations in the structural and chemical heterogeneity of such dendrimers. For example, each generation must comprise of the same kind of chemical entities.

Recently there has been work on macromolecules whose branched chains are macromolecular segments as opposed to small molecular units. These require a multifunctional core initiator from which the polymer arms are grown to give a dendrimer like molecule. However, there are limitations to this synthetic method and it is not possible to build up layers or generations of macromolecule segments. Further limitations in the structural and chemical heterogeneity of the macromolecule remain.

Therefore there remains a need for dendritic molecules which retain the advantageous properties and controlled structure of a dendrimer whilst providing chemical and structural heterogeneity and precise surface and interior functionalisation. Importantly, there remains a need to synthesise such dendritic molecules by way of a reasonably small number of versatile and reliable step-wise reactions.

It is, accordingly, an object of the present invention to overcome Of at least alleviate one or more of the difficulties and deficiencies related to the prior art.

It will be understood that the invention disclosed and defined in this specification extends to all alternative combinations of two or more of the individual features mentioned or evident from the text or drawings. All of these different combinations constitute various alternative aspects of the invention.

### SUMMARY OF THE INVENTION

In a first aspect, the present invention relates to a dendron comprising at least three arms wherein each of the arms is a preformed polymer and wherein at least one of the arms comprises a functional group having an active site capable of bonding to one or more preformed polymers thereby to form a further generation.

In a second aspect, the present invention relates to a dendron comprising:

a first polymer;

one or more first generational polymers bound to the first polymer; and

wherein the first generational polymers include a functional group having at least one active site capable of bonding to a predetermined number of one or more further generational polymers.

In a third aspect the present invention relates to a dendron comprising a first polymer, one or more first generational polymers bound to the first polymer and one or more further generational polymers extending outwardly from the one or more first generational polymers.

The polymers of the dendrons of the invention may be linear or branched. Further, each generation is composed of the same or different polymers.

In a fourth aspect the present invention relates to a dendritic molecule comprising two or more dendrons wherein each arm of each of the dendrons is a preformed polymer.

In a fifth aspect the present invention relates to a dendritic molecule comprising two or more dendrons bound together by a common multifunctional group, each dendron comprising:

a first polymer;

one or more generational polymers bonded to the first polymer; and

optionally a predetermined number of further generational polymers extending outwardly from the first generational polymers.

Preferably the dendritic molecule includes a predetermined number of further generational polymers extending outwardly from the first generational polymers.

Preferably the two or more dendrons are bound together by a common multifunctional group and each dendron includes a first polymer, one or more first generational polymers bonded to the first polymer and a predetermined number of further generational polymers extending outwardly from the first generational polymers.

In a sixth aspect the invention relates to a dendritic molecule comprising:

a first polymer comprising two or more functional groups having at least one active site;

two or more generational polymers bonded to the active sites to form a first generational macromolecule, each of the first generational polymers comprising two or more functional group having an active site; and

optionally a predetermined number of further generational polymers extending outwardly from the first generational polymers.

In a seventh aspect the invention relates to a dendritic molecule comprising:

a core or first polymer that is a star polymer comprising three or more arms, at least one arm comprising a functional group having an active site; and

one or more generational polymers or one or more dendrons bound to the active site.

Preferably the dendritic molecule is a mikto-arm dendrimer.

In a eighth aspect the present invention relates to a method of forming a dendron comprising the steps of coupling three or more preformed polymer arms thereby to form the dendron and wherein at least one of the arms of the dendron comprises a functional group having an active site capable of bonding to one or more preformed polymers thereby to form a further generation.

In a ninth aspect the present invention relates to a method of forming a dendron comprising the steps of:

(a) forming a first polymer comprising a functional group having at least one active site;

(b) bonding at least one first generational polymer to the at least one active site of the first polymer to form a first generational macromolecule; and

(c) wherein the first generational polymer includes a functional group having at least one active site capable of bonding to at least one further generational polymer.

In a tenth aspect the present invention relates to a method of forming a dendron comprising the steps of

(a) forming a first polymer;

(b) bonding a functional group having at least one active site to the first polymer;

(c) bonding at least one generational polymer to the at least one active site of the first polymer to form a first generational macromolecule;

(d) bonding a functional group having at least one active site to at least one site on the first at least one generational polymer of the macromolecule to provide at least one active site on the macromolecule; and

(e) bonding at least one further generational polymer to the at least one active site on the macromolecule; and

(f) repeating steps (d) and (e) until a predetermined number of generational polymers have been added.

In a eleventh aspect the invention relates to a method of forming a dendritic molecule comprising the steps of coupling two or more dendrons wherein each arm of each of the dendrons is a preformed polymer.

Preferably the dendrons are prepared according to the eight, ninth or tenth aspect of the invention.

In a twelfth aspect the invention relates to a method of convergently forming a dendritic molecule comprising the steps of

(a) forming a plurality of dendrons, each dendron being formed by the steps of
(1) forming a first polymer,
(2) bonding a functional group having at least one active site to the polymer,
(3) bonding at least one generational polymer to the at least one active of the polymer to form a first generational macromolecule,
(4) bonding a functional group having at least one active site to the at least one generational polymer end of the macromolecule,
(5) bonding at least one further generational polymer to the at least one active site of the macromolecule to provide an active site on the macromolecule, and
(6) repeating steps (4) and (5) until a predetermined number of generational polymers have been added, and

(b) bonding a multifunctional group having two or more active sites to the non-functionalised end of the first polymer and bonding two or more dendrons to the active sites of the multifunctional group bonded to the first polymer.

In a thirteenth aspect of the invention there is provided a method of forming a dendritic molecule comprising the steps of

forming a first polymer comprising two or more functional groups having at least one active site;

bonding two or more first generational polymers with the active sites to form a first generational macromolecule thereby forming a first generational macromolecule wherein the first generational polymer comprises two or more functional groups having at least one active site; and

optionally iteratively bonding further generational polymers to the active site on the first generational macromolecule, each iterative step resulting in a generational macromolecule having a functional group with an active site until termination.

In a fourteenth aspect of the invention there is provided a method of divergently forming a dendritic molecule comprising the steps of:
(a) forming a first polymer
(b) bonding two or more functional groups having at least one active site to the first polymer;
(c) bonding two or more generational polymers to the active sites on the first polymer to form a first generational macromolecule;
(d) bonding one or more functional groups having at least one active site to a plurality of sites on the first generational macromolecule;
(e) repeating steps (c) and (d) until a predetermined number of generational polymers have been added.

In a fifteenth aspect the invention relates to a method of forming a dendritic molecule comprising the steps of forming a star polymer each of whose arms comprises a functional group having an active site and bonding one or more dendrons to the active site.

In a sixteenth aspect the invention relates to a delivery molecule comprising a dendron or dendritic molecule and one or more active molecules, wherein the active molecule(s) are bound to the polymeric arms by a degradable or cleavable linkage.

Preferably the dendron or dendritic molecule has polymeric arms

Preferably the dendritic molecule is comprised of dendrons wherein each arm of the dendrons is a preformed polymer. Preferably the active is linked to the pendant groups of the preformed polymer. Preferably the linkage is biodegradable.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

### BRIEF DESCRIPTION OF FIGURES & SCHEMES

FIG. 1: Attenuated total reflectance FT-IR spectra of 4-vinylbenzene chloride crosslinked beads [39], propargyl functionalized crosslinked beads [40] and azide functionalized crosslinked beads [41] of Example 5.

FIG. 2: Size exclusion chromatograms using refractive index detection of PSTY-(—≡)₂ [28], Dendron-G₀-G₁-PSTY-Sol [42], Dendron-G₀-G₁-PSTY-Sol [42]* and Dendron-G₀-G₁-G₂-PSTY-Sol [46] (*after reaction with crosslinked beads [40]) of Example 6.

FIG. 3: Size exclusion chromatograms using refractive index detection of PSTY-(—≡)₂ [28], Dendron-G₀-G₁-PSTY-Sol [42]* and Dendron-G₀-G₁-G₂-PSTY—(OH)₂ [47] (*after reaction with crosslinked beads [40]) of Example 6.

FIG. 4: Size exclusion chromatograms using refractive index detection of PSTY-(—≡)₂ [28], Dendron-G₀-G₁-PSTY-Sol [42]* and Dendron-G₀-G₁-PSTY-G₂-P^{t}BA-(OH)₂ [48] (*after reaction with crosslinked beads) of Example 6.

FIG. 5: Size exclusion chromatograms using refractive index detection of Dendron-G₀-G₁-G₂-PSTY-Sol [46] of Example 6 and after degradation reaction with NaOCH₃.

FIG. 6: Size exclusion chromatograms using refractive index detection of (≡—)₂—PSTY-(—≡)₂ [29], Sym-G₀-G₁-PSTY-Sol [49]* and Sym-G₀-G₁-G₂-PSTY-Sol [53]* (*after reaction with functionalized crosslinked beads [41]) of Example 7.

FIG. 7: Size exclusion chromatograms using refractive index detection of (≡—)₂—PSTY-(—≡)₂ [29], Sym-G₀-G₁-PSTY-Sol [49]* and Sym-G₀-G₁-G₂-PSTY—(OH)₂ [54] (*after reaction with functionalized crosslinked beads [40]) of Example 7.

FIG. 8: Size exclusion chromatograms using refractive index detection of (≡—)₂—PSTY-(—≡)₂ [29], Sym-G₀-G₁-PSTY-Sol [49]* and Sym-G₀-G₁-G₂-P^{t}BA-(OH)₂ [55] (*after reaction with functionalized crosslinked beads [40]) of Example 7.

FIG. 9: Size exclusion chromatograms using refractive index detection of (≡—)₂—PSTY-(—≡)₂ [29], Sym-G₀-G₁-PSTY-Sol [49]* and Sym-G₀-G₁-PSTY-G₂-PMA-(OH)₂ [56] (*after reaction with functionalized crosslinked beads [40]) of Example 7.

FIG. 10: Size exclusion chromatograms using refractive index detection of Sym-G₀-G₁-G₂-PSTY-Sol [53] of Example 7 and after degradation reaction with NaOCH₃.

FIG. 11a-11d: Size exclusion chromatograms of Example 8 using refractive index detection of HO—PSTY—Br [15], HO—PSTY—(PSTY)₂ [58] and —(PSTY—(PSTY)₂)₃ [68]. ^{f}After fractionation by SEC. (a) [58] and [68] prepared by Method A: 10×CuBr/PMDETA, (b) [58] and [68] prepared by Method B: 0.5×CuBr/PMDETA, (c) [58] and [68] prepared by Method C: Cu (wire) and (d) [68] prepared by Method C from starting functional stars prepared by Method B.

FIG. 12a-12c: Size exclusion chromatograms of Example 8 using refractive index detection of HO—PSTY—Br [15], HO—PSTY—(PSTY)₂ [58] and (PSTY)₂—PSTY—(PSTY—(P^{t}BA₂))₂ [69]. After fractionation by SEC. (a) [58] and [69] prepared by Method A: 10×CuBr/PMDETA, (b) [58] and [69] prepared by Method B: 0.5×CuBr/PMDETA and (c) [69] prepared by Method C from starting functional stars prepared by Method B.

FIG. 13a-13c: Size exclusion chromatograms of Example 8 using refractive index detection of HO—PSTY—Br [15], HO—PSTY—(PSTY)₂ [58] and (PSTY)₂—PSTY—(PSTY—(PMA)₂)₂ [70]. ^{f}After fractionation by SEC. (a) [58] and [70] prepared by Method A: 10×CuBr/PMDETA, (b) [58] and [70] prepared by Method B: 0.5×CuBr/PMDETA and (c) [70] prepared by Method C from starting functional stars prepared by Method B.

FIG. 14: Size exclusion chromatograms of Example 8 using refractive index detection of —(PSTY—(PSTY)₂)₃ [68] and after degradation reaction with NaOCH₃.

FIG. 15: Size exclusion chromatograms using refractive index detection of G2[G1PSTY—N₃, G2PSTY₂] [64], Star P(^{t}BA₁₁₇-(≡)₂)₄ [73b] and G3[G1P(AA₃₇)₄, G2PSTY₈, G3PSTY₁₆] [77a].

FIG. 16: SDS-PAGE

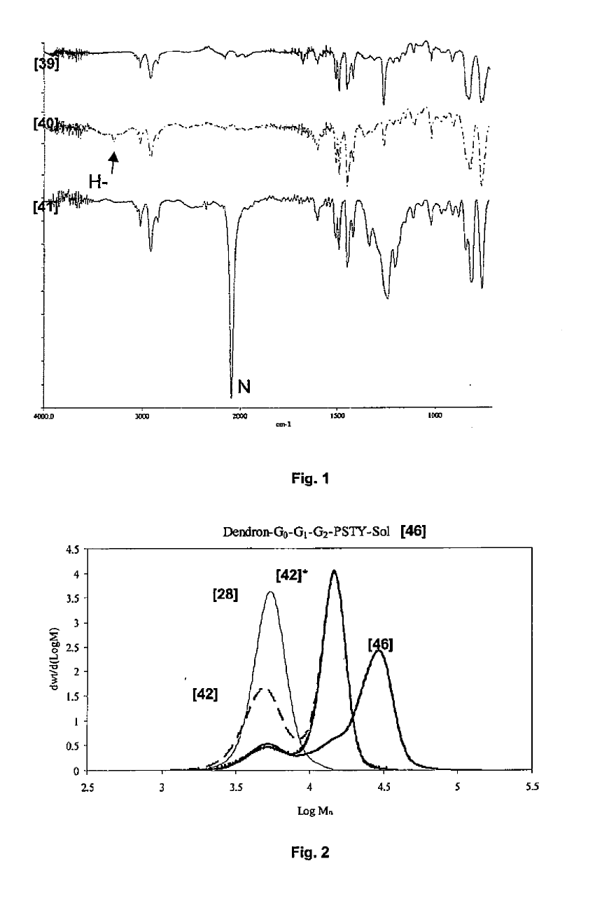

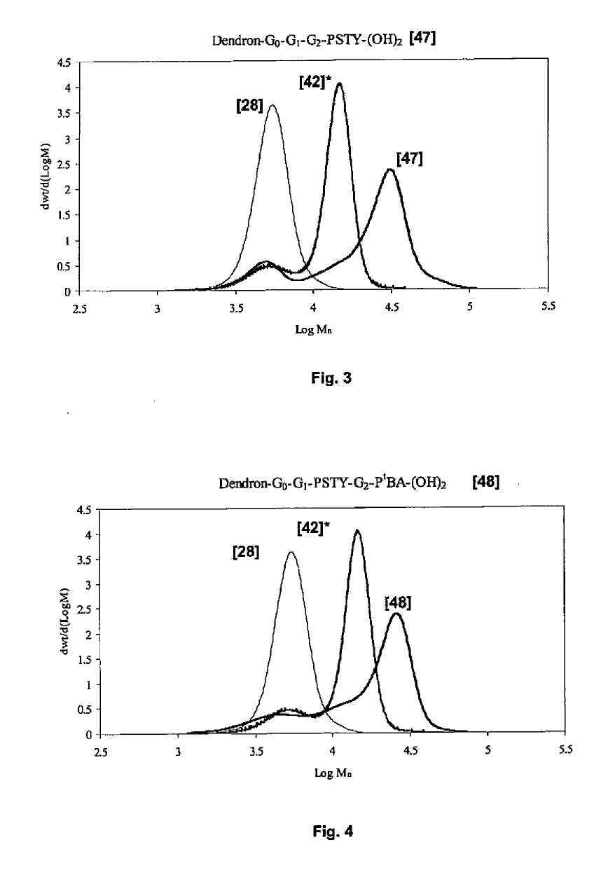

Scheme 1: Synthesis of 4-arm stars by ATRP at 35° C. 71a (Mₙ=19000 and PDI=1.09) and 71b (Mₙ=60000 and PDI=1.11).

Scheme 2: Methodology to make reactive PSTY dendrons.

Scheme 3: Synthesis of 3^{rd} generation dendrimer where the 1^{st} generation consists of PtBA and the 2^{nd} and 3^{rd} generation consists of PSTY.

Scheme 4: Coupling siRNA to dendrimer.

### DETAILED DESCRIPTION OF THE INVENTION

In general, a dendrimer has well-regulated branch structures which extend three-dimensionally from a core. Dendrons are usually dendrimer sections which extend in one direction from a core. The terms “extend”, “extend outwardly” are well known in dendrimer art and are not defined further.

The term dendritic molecule in the text is usually used interchangeably with the term dendrimer, however it is to be understood that the term can also be used interchangeably with the term dendron.

The term polymer as used throughout this specification is any macromolecule having multiple repeat units. The term therefore includes oligomers. The polymer can be linear or branched. Branched polymers include those conventionally known in the art.

The term polymer when used to define the structure of the dendritic molecule can also be understood as commonly known terms of dendrimer art like “arms”, “dendrite”, and “branch”, “segment” and the like, Similarly the term “generation” can be used interchangeably with the term “layer” and the like.

The term generation is as understood in dendrimer art. Each generation has twice as many branch points as the previous generation.

The term “functional arm star” may also be used for the dendron.

Conventional small molecule dendrimers follow the nomenclature proposed by Newkome. In this text, the nomenclature depends upon the method of making the dendron or dendrimer. The numbering is therefore flexible but the naming follows the convention of successive numbering. In general the core or the starting polymer is usually termed Generation 0 or G₀. Subsequent generations are defined as Generation 1, 2, 3 and so on (G₁, G₂, G₃ etc.). Each generation comprises polymer arms which are designated by way of a suffix to G₁, G₂ etc. as P, Pₐ, P_{b} and so on. Like with conventional dendrimer structures, the diameter and the number of arms increases linearly with each generation.

Throughout the specification there is reference to the bonding of a functional group having an active site to a polymer or dendron. The term is intended to indicate that the functionalisation of a polymer or dendron can result from polymerisation or dendronisation or functionalisation can be introduced post polymerisation or dendronisation.

The present invention relates to a novel dendron comprising at least three arms wherein each of the arms is a preformed polymer and wherein at least one of the arms comprises a functional group having an active site capable of bonding to one or more preformed polymers thereby to form a further generation.

Typically the dendron comprises a first polymer and one or more fast generational polymers bound to the first polymer. The first generational polymer includes a functional group having at least one active site capable of bonding to one or more further generational polymers. The further generational polymers extend outwardly from the one or more first generational polymers.

In its simplest arm the invention relates to a three-arm dendron. This differs from conventional three arm star polymers in that at least one arm has an active site that can form the next generation of the dendron.

The core or “generation 0” (G₀) of the dendron is the first polymer to which is bonded a functional group having an active site. A single functional group or multiple functional groups can be bonded either post polymer formation or by way of polymer formation. Further, each functional group can have one or more active sites. The functional groups may be terminal or located along the length of the polymer chain.

In its simplest form, the first polymer is a linear polymer. In other embodiments the first polymer can be a branched polymer. Similarly the first generational polymer and subsequent generational polymers can also be linear or branched polymers.

The first polymer can be coupled or bonded with a generational polymer thereby to give a first generation or G₁. The resulting three arm dendron can be represented as G₀-G₁-P—X wherein G₀ is the first polymer, G₁ is the first generation comprising two or more polymer P arms and X is a functional group having an active site that is capable of bonding to the next generational polymer to form the next generation i.e. G₂. When the next generation is formed the structure is represented as G₀-G₁-Pₐ-G₂-P_{b}—X wherein G₀ is the first polymer, G₁ is the first generation comprising polymer Pₐ arms, G₂ is the second generation comprising polymer P_{b} arms and X is a functional group having an active site that is capable of bonding to further generational polymers.

A similar nomenclature as above is followed as successive generations are added.

Pₐ and P_{b} may be the same or different. Further, it is understood that the number of arms will increase in each successive generation i.e. G₂ will comprise more polymer arms than G₁.

More than one functional group X can be present on the polymer arms of each generation and each functional group can have one or more active sites. As with the first polymer, the functional groups may be terminal or located along the length of the polymer chain. The functional groups on each generation are such as to provide twice the number of branch points as the previous generation.

The invention also relates to a dendritic molecule, which has a first polymer comprising two or more functional groups having at least one active site. Two or more generational polymers are bonded to the active sites to form a first generational macromolecule, each of the first generational polymers having two or more functional group having an active site. A predetermined number of further generational polymers extend outwardly from the first generational polymers. In some embodiments the first polymer has a functional group having an active site at both terminal ends. When bonded to the first generational polymer, a symmetrical first generational macromolecule is formed. The resulting dendrimer can be represented as Sym-G₀-G₁-P—X wherein G₀, G₁, P and X are as above. Further generational polymers can be added as discussed earlier. A similar nomenclature is used for the resulting dendrimers e.g. Sym-G₀-G₁-Pₐ-G₂-P_{b}—X when a second generation comprising polymer P_{b} arms is formed. As before Pₐ and P_{b} may be the same or different.

A similar nomenclature as above is followed as successive generations are added.

Thus the polymers in the first generational layer and in each further generational layers can be the same polymers or different polymers. Hence each generational polymer and consecutive generational layers may contain the same or different polymers. The polymers used will depend on the requirements of the resulting dendron and/or dendrimer, in terms of chemical composition, chemical functionality and size. Additionally, the fast polymer, the first generational layer and each subsequent generational layer may be functionalised in the same way or in a different way.

The invention also relates to a dendritic molecule comprising two or more dendrons wherein each arm of each of the dendrons is a preformed polymer.

The dendrons are as described above i.e. each dendron includes a first polymer, one or more first generational polymers bonded to the first polymer and optionally a predetermined number of further generational polymers extending outwardly from the first generational polymers. The dendrons are bound or coupled together to form a dendrimer. Preferably the two or more dendrons are bound or coupled together by a common multifunctional group.

A first dendron of such a dendritic molecule or dendrimer can be as discussed earlier. However, the first polymer is a preformed polymer that has a functional group having two or more active sites on its non-functionalised end. The other dendrons are also synthesised as discussed earlier, however the first polymer is a preformed polymer having a functional group with one active site at its non-functionalised end.

The dendrons may be the same or different. When the dendrons are different, it is possible to obtain mikto-arm or “mixed” arm star dendrimers. Structural heterogeneity within each generation of a dendritic molecule is hitherto unknown.

A simple way of representing the dendrons or functional arm stars which can be coupled to form the dendrimer is G₂[G₁Pₐ—X, G₂P_{b}] where each of G₁, Pₐ, X, G₂ and P_{b} have the same meaning as before. In this nomenclature, the first polymer is taken as G₁ in order to clearly indicate the functional group with an active site at its proximal end i.e. X. Again Pₐ and P_{b} may be the same or different. A similar nomenclature as above is followed as successive generations are added.

A first dendron having at least two active sites at the non-functionalised end of the first polymer can be coupled with two dendrons, each of which has a functional group with an active site, to form a dendrimer that is a three arm dendritic star. The polymer arms Pₐ of the first dendron can be different from the P_{b} arms of the two dendrons bonded to the first dendron thereby generating mikto-arm dendrimers. Such structures are hitherto unknown.

Another dendritic molecule comprises a core or first polymer that is a star polymer comprising three or more arms, with at least one arm comprising a functional group having an active site. One or more first generational polymers or one or more dendrons are bound to the active site.

In one embodiment of the “star” dendrimer, a star polymer has one or more first generational polymers bonded to each of its arms. Each generational polymer can optionally carry a predetermined number of further generational polymers extending outwardly from the first generational polymer. Alternatively, the dendrimer comprises a star polymer to each of whose arms is bonded a dendron. Such dendrons may be of the type described above.

In the most preferred embodiments, the star polymer is prepared from a multifunctional initiator and has one or more functional groups with at least two active sites bonded to each arm of the star polymer. Each arm can be bound or coupled to two or more dendrons G₂[G₁Pₐ—X, G2P_{b}] where X is a functional group having at least one active site bonded to the non-functionalised end of the first polymer. Third generation dendrimers can therefore be obtained by way of a small number of reactions. In this text, such dendrimers are represented as G₃[G₁Pₐ, G₂P_{b}, G₃P_{c}], Pₐ, P_{b} and P_{c} may be the same or different. As before, when the two or more dendrons are different, it is possible to obtain mikto-arm or “mixed” arm star dendrimers having structural heterogeneity within each generation of a dendrimer or dendritic molecule.

The two or more dendrons making up any of the dendritic molecules of the invention may be the same or different. Where the two or more dendrons are the same, the dendritic molecule will be symmetrical. Where the dendrons are different, the dendrons may have a different chemical composition, different chemical functionality, and/or different chain lengths. Where the dendrons in the dendritic molecule are different, the dendritic molecule may include two or more different dendrons. Where the dendrons in the dendritic molecule are different, the resulting dendritic molecule may be asymmetric in a number of ways, including, but not limited to, asymmetric in terms of function, polarity, hydrophobicity (amphipathic), or generation number).

It will be appreciated that in general bonding between any of the polymers/dendrons discussed above and a functional group comprising an active site may be direct or by way of a linker or spacer molecule. Similarly the bond between any of the polymers/dendrons discussed above may be direct or by way of a linker or spacer molecule. The choice of the linker or spacer molecule will depend upon a number of factors including the kind of polymer and functional group.

The polymers/dendrons as discussed above can include one or more functional groups that do not participate in bonding or coupling. Such a group can be terminal or be present on any site along the length of the polymer. Where appropriate such a group can be protected by conventional methods in the art and then deprotected when required. Such a functional group can be bonded directly to the polymer/dendron or by way of a linker. However, such functional groups can include active sites capable of facilitating bonding or coupling in subsequent reactions, examples of which include solketal, hydroxyl and halogen groups.

The methods for preparing the dendrons and dendrimers according to the invention will now be discussed in detail.

In the present invention three or more preformed polymer arms are coupled to form the dendron. At least one of the arms of the dendron comprises a functional group having an active site capable of bonding to one or more preformed polymers thereby to form a further generation.

A method of preparing dendrons for the formation of a dendritic molecule comprises the steps of forming a first polymer comprising a functional group having at least one active site and bonding at least one first generational polymer to the at least one active site of the first polymer to form a first generational macromolecule. The first generational polymer includes a functional group having at least one active site capable of bonding to at the next generational polymer.

For example, a three-arm dendron can be prepared by bonding two generational polymers to a first polymer having two active sites.

A functional group having an active site is bonded to a site on the aforesaid first generational polymer of the macromolecule to provide an active site on the macromolecule and at least one further generational polymer is then bonded to the at least one active site on the macromolecule to form the next generation. The father generational polymer can be a dendron thereby forming two or more generations by way of a single bonding or coupling reaction. This step can be repeated to provide further generations.

For example, each of the two polymer arms (G₁) of the aforesaid three arm dendron may have a functional group with an active site, X. This can be a “precursor” active site which has to be appropriately functionalised before bonding to the further generational polymer or may be an active site itself capable of bonding with the further generational polymer. For example, each polymer arm of G₁ can carry two active sites which can bond to the further generational molecule to form the polymer arms of G₂ thereby giving G₀-G₁-Pₐ-G₂-P_{b}—X. This step can be repeated until a predetermined number of generations are obtained.

As will be appreciated, the above iterative steps can also be applied to a first polymer, which is functionalised at both ends. Since the final structure is symmetrical, a dendrimer is obtained. Similarly the above iterative steps can also be applied to a star polymer having functionalised arms to obtain a dendrimer. Further, the invention provides for methods of forming dendritic molecules either divergently or convergently or by combining both approaches.

In the convergent method of forming a dendritic molecule, two or more dendrons are coupled or reacted together to form a dendritic molecule. Each arm of the two or more dendrons is a preformed polymer. Each dendron is formed in accordance with the invention. A functional group having two or more active sites is then bonded to the non-functionalised end of the first polymer of a first dendron or may be present on the first dendron. Two or more dendrons are then bonded to the active sites of the functional group bonded to the first polymer. Therefore the dendron “wedges” constituting the periphery and interior are formed first and then coupled to form a core. G₂[G₁Pₐ—X, G₂P_{b}] dendrons or functional arm stars in particular can be reacted or coupled convergently to form dendrimers or dendritic stars.

Preferably three or more dendrons are bound to the active sites of the (multi)functional group bonded to the first polymer, more preferably four or more dendrons are bound to the active sites, most preferably five or more dendrons are bound to the active sites.

In the divergent method of forming a dendritic molecule, a first polymer comprising two or more functional groups having at least one active site is formed and two or more generational polymers are bonded or reacted with the active sites to form a first generational macromolecule. Each of the first generational polymers comprises two or more functional groups having an active site. The steps are repeated with a predetermined number of further generational polymers which carry two or more functional groups having an active site until termination. The iterative coupling forms the dendritic molecule.

The two or more functional groups having at least one active site may be bonded to the polymer or may be present on the polymer. To these active sites is bonded two or more generational polymers to form a first generational macromolecule. One or more functional groups having at least one active site are then bonded to a plurality of sites on the first generational macromolecule or are present on these sites. Further iterative coupling of a predetermined number of generational polymers forms the dendritic molecule. Symmetrical dendrimers as well as star dendrimers discussed earlier may be prepared by this method.

Preferably the two functional groups are at the terminal ends of the first polymer or at the terminal end of the star polymer or first generational polymers.

A combination of methods may also be used. For example when a star dendrimer is formed by bonding dendrons to a star polymer, the star polymer itself is formed divergently. However, the bonding or coupling between the star polymer and dendron is more akin to a convergent approach as the periphery is first formed and then the interior of the dendrimer.

The methods of forming dendrons and dendritic molecules described above may include the use of protecting groups. Suitable protecting groups would be known to the person skilled in the art.

At any stage in the method of forming a dendritic molecule or dendron, any unreacted polymer is easily separated by binding the same to an appropriately functionalised cross-linked polymeric bead. When building a dendritic molecule, preferably this step is repeated after each bonding or coupling step. Thus its possible to obtain a substantially pure dendron or dendritic molecule. However, a small amount of unreacted polymers and/or reagents may be present without affecting the properties of the dendron or dendritic molecule.

It is clear from the description as a whole that the dendron/dendritic molecule of the invention is formed by bonding or reaction between a polymer or dendron having a functional group carrying an active site with another polymer or dendron having a functional group carrying an active site. Such functional groups having an active site may be any functional group known to the person skilled in the art. In some embodiments, the bonding or reaction may also take place via a linker. Such a linking group may be any suitable bifunctional chemical moiety known to a person skilled in the art. Similarly in some embodiments the polymer or dendron may be bonded to the functional group having an active site via a linker, which is a suitable bifunctional chemical moiety.

Typically such functional groups include, but are not limited to those that are complementary and capable of reacting together to form a stable bond. Further the functional groups require to be selected such that each generation will comprise more arms than the previous so as to build the required dendritic molecule structure.

Preferably the functional groups are able to participate in pericyclic reactions. Pericyclic reactions are a type of organic reaction wherein the transition state of the molecule has a cyclic geometry, and the reaction progresses in a concerted fashion. Pericyclic reactions include, amongst others, electrocyclic reactions, cycloadditions, sigmatropic rearrangements and group transfer reactions. Common examples of pericyclic reactions include the Diels-Alder reactions, e.g. between maleimides and furans and “click” chemistry reactions. The click chemistry approach and the possible click reactions are discussed in H. C. Kohl, M. G. Finn and K. B. Sharpless, Angew. Chem. Int. Ed., 2001, 40, 2004-2021 included herein by reference. Ideally these reactions would be modular, wide in scope, high-yielding, create inoffensive by-products that are readily removed, simple to form and require benign or easily removed solvents. Preferably the reactions occur under mild conditions, give rise to few by-products and approach 100% yields.

The click chemistry strategy relies mainly upon the construction of carbon-heteroatom bonds using spring-loaded reactants. Several processes are considered especially suitable for click chemistry including cycloadditions of unsaturated species, Diels-Alder family of transformations, nucleophilic substitution chemistry including ring-opening reactions of strained heterocyclic electrophiles such as epoxides, aziridines, aziridiniumions, and episulfoniumions, carbonyl chemistry of the non-aldol type, such as formation of ureas, thioureas, aromatic heterocycles, oxime ethers, hydrazones, and amides, and additions to carbon-carbon multiple bonds including oxidative cases such as epoxidation, dihydroxylation, aziridination, and sulfenyl halide addition, Michael additions of Nu-H reactants and 1,3 dipolar cycloaddition reactions.

Examples of functional groups which are complementary are hydroxy groups and carboxylic acid groups (which produce ester bonds), amines and carboxylic acid groups (which produce amide bonds), epoxide groups and amine groups (which will produce C—N bonds), thiols and Michael acceptors (which produce C—S bonds), hydrosilation reaction of H—Si and simple non-activated vinyl compounds, urethane formation from alcohols and isocyanates, Menshutkin reaction of tertiary amines with alkyl iodides or alkyl trifluoromethanesulfonates, Michael additions chemistry reaction groups and the like. In some cases, such as vinyl groups, the complementary functional groups may be identical.

Especially preferred reaction is a ‘click’ chemistry approach. An example is the Azide-Alkyne Huisgen Cycloaddition or 1,3-dipolar cycloaddition between an azide and a terminal or internal alkyne to give a 1,2,3-triazole. A preferred variant of the Huisgen 1,3-dipolar cycloaddition is the copper(I) catalyzed variant, in which organic azides and terminal alkynes are coupled to afford 1,4-regioisomers of 1,2,3-triazoles as sole products from complementary functional groups azides and alkynes. A particularly preferred exemplification of the alkyne for the present invention is a tripropragyl or dipropargyl moiety.

The copper catalyst used may include, but is not limited to, commercial sources of copper or copper (I) including copper wire, copper shavings, copper (I) bromide, copper (I) iodide; or a mixture of copper (II) and a reducing agent which produces copper (I) in situ, for example, a mixture of copper (II) sulphate and sodium ascorbate. Especially preferred is copper wire since it may easily be removed after the reaction is completed.

The copper catalysed reaction between the azide-moiety and the alkyne moiety may be performed in the presence of a ligand. Where a ligand is used, the ligand may be selected from N-(n-propyl)pyridylmethanimine (NPPMI), N-(n-octyl)pyridylmethanimine (NOPMI), Tris(2-(dimethylamino)ethyl)amine (Me6TREN), 4,11-dimethyl-1,4,8,11-tetraazabicyclo[6.6.2]hexadecane (Cyclam-B), 4,4′-di(9-heptadecyl)-2,2′-bipyridyne (dHDbpy), 4,4′-di(5-nonyl)-2,2′-bipyridyne (dNbpy), 4,4′,4″-tris(5-nonyl)-2,2′:6′,2″-terpyridine (tNtpy), N,N-bis(2-pyridylmethyl)octadecylamine (BPMODA), tris-[(2-pyridyl)methyl]amine (TPMA), N,N,N′,N′-tetramethylethylenediamine (TMEDA), 1,4,8,11-tetramethyl-1,4,8,11-tetraazacyclotetradecane (Me4Cyclam), N,N,N′,N′,N″-pentamethyldiethylenetriamine (PMDETA), 1,1,4,7,10,10-hexamethyltriethylenetetramine (HMTETA), 2,2′-bipyridyne (bpy) and 1-methyl-8-ammine-3,13,16-trithia-6,10,19-triazabicyclo[6.6.6]icosane (NH2capten). Especially preferred is PMDETA.

The functional groups having at least one active site referred to above may be added to or be present on any position of the polymer/dendron as required. For example, the functional group may be added to one of the ends of the first polymer or the distal end of the generational polymer(s) or to any position along the length of the first polymer or the generational polymer(s). Where the functional group is added to a site other than the end of the polymer, the functional group forms a side group off the main polymer structure. These active sites then form the sites for bonding the next generational polymer. When two or more active sites are formed on the end of each polymer, the dendron produced has a branched structure.

The polymers of the dendritic molecules of the invention i.e. the arms or segments of the molecule can be prepared by known polymerisation techniques. These include, but are not limited to, addition polymerisation (including anionic and cationic polymerisation), chain polymerisation, free radical or ‘living radical’ polymerisation (including atom transfer radical polymerisation or ATRP), metal catalysed, nitroxide, degenerative chain transfer, Reversible Addition-Fragmentation chain Transfer polymerisation (RAFT), SET-LRP and condensation polymerisation. Especially preferred is ATRP which provides controlled polymerisation and end products with low polydispersity. ATRP commonly uses a transition metal catalyst in a small amount and has the ability to polymerise a wide variety of monomers, Polymers produced by ATRP methods often contain a terminal halogen atom at the growing chain end which can be efficiently modified in various end-group transformations, replacing terminal halogen for example, with azides, amines, phosphines and other functionalities.

Examples of polymers that may be conveniently synthesised by ATRP include polystyrene, polyacrylates and the like.

The first polymer and the generational polymers may be of any suitable molecular size or weight depending on the requirements of the dendron and dendritic molecule. Where required they may also be oligomers. Preferably, the polymers have more than 5 repeating units, more preferably the polymers have more than 10 repeating units, most preferably, the polymers have more than 20 repeating units.

Where required it is also possible to degrade or break down the dendritic molecule into smaller discrete elements. This is useful for example in pharmaceutical applications where it may be required that the dendritic molecule break down within the body to facilitate delivery of actives. In some embodiments the polymer arm itself may be a biodegradable polymer, in other embodiments, the linkages between the polymer arms are degradable.

The pendant groups of the polymer arms of dendrons or dendrimers can also be deprotected if required. In particular, where polymer arms are a polyacrylate, the acrylate groups can be easily converted to the corresponding acid. Such acrylic acid polymer containing dendrimers can micellise to form amphiphilic dendrimers.

The polymers and starting monomers of the present invention are now described in greater detail.

The first polymer and the first generational and further generational polymers may be of any suitable type known to the person skilled in the art and may be selected depending on the requirements of the resulting dendron and/or dendrimer. For example, the polymer may be a homopolymer (a polymer made up from identical monomers), a gradient polymer, or a co-polymer (a polymer made up of two or more chemically different monomers. The co-polymer may be a “block copolymer” (a copolymer in which the repeating units in the main polymer chain occur in blocks) or a “graft co-polymer” (a polymer that consists of homopolymeric branches joined or grafted to another homopolymer). The polymer may be linear (a polymer whose molecules form long chains without cross-linked or branch structures) or branched (a polymer having side-chains extending from the polymer backbone). Where the polymer is branched, the polymer may be of any suitable type, including, but not limited to, a star-branching polymer (a polymer where the branches ultimately emanate from a single point), or a dendrimer, also known as cascade polymers (a polymer with a high degree of branching, where the branches themselves are typically also further branched). The polymer may also be a biodegradable polymer (such as a biodegradable polylactic acid), a biocompatible polymer (eg, PEG), or a polymeric biomolecule (including, but not limited to, a carbohydrate, a saccharide chain, a protein, a polypeptide, a peptide, a form of DNA, a form of RNA, or other nucleic acid, such as PNA).

The term “block polymer” as used herein refers to a block copolymer containing two or more polymerised blocks of sections of like monomer. The block copolymers may be diblock copolymers, or may have three or more blocks. Each block may be different or the blocks may alternate.

The block copolymers useful in accordance with the present invention are generally diblock polymers of formula -(A)ₘ(B)ₙ— where A represents the polymerised residue of the monomer of one block, B represents the polymerised residue of the monomer of the second block, and in and n represent the number of repeat units of monomers A and B respectively

In preferred embodiments, at least one block of the block copolymers of the present invention should be synthesised using living/controlled free radical polymerisation. More preferably the whole block copolymer is synthesised using living/controlled (free radical) polymerisation. It is to be understood that the nature of the end groups of the block polymers of the present invention will depend on the nature of the initiators used, and the type of living/controlled free radical polymerisation employed, and the desired functionality.

The term “graft polymer” as used herein refers to a graft polymer comprising a polymeric backbone, which may be of one monomer type or may be a block copolymer, to which a further polymeric chain, which may also be of one monomer type or may be a block copolymer, is grafted, usually through pendant reactive or polymerisable groups present on the polymeric backbone, or through unsaturation in the polymeric backbone. The polymeric backbone is prepared using living/controlled free radical polymerisation techniques. The grafted polymer may be introduced using any suitable technique. The polymer to be grafted may be prepared separately and attached to the polymeric backbone through reaction of a reactive group present on the graft polymer with a complementary reactive group on the backbone. The term “complementary” as used herein when referring to functional groups means that two functional groups are capable of reacting together to form a stable bond. Examples of functional groups which are complementary are hydroxy groups and carboxylic acid groups (which will produce ester bonds) epoxide groups and amine groups (which will produce C—N bonds), thiols and Michael acceptors (which will produce C—S bonds) and the like. In some cases, such as vinyl groups, the complementary functional groups can be identical. A person skilled in the art would be able to select appropriate functionalities to attach the graft to the backbone. In another embodiment the graft polymer is polymerised onto the polymeric backbone using a suitable polymerisation technique.

Where the polymer is conjugated, the dendritic molecule can fund application in a light-emitting device.

Preferably, one or more of the polymers or a part thereof is composed of a biodegradable polymer.

The polymers as described above may be formed from any suitable monomer(s) known to the person skilled in the art, including, but not limited to, at least one monomer selected from the group consisting of styrene, substituted styrene, alkyl acrylate, substituted alkyl acrylate, alkyl methacrylate, substituted alkyl methacrylate, acrylonitrile, methacrylonitrile, acrylamide, methacrylamide, N-alkylacrylamide, N-alkylmethacrylamide, N,N-dialkylacrylamide, N,N-dialkylmethacrylamide, isoprene, 1,3-butadiene, ethylene, vinyl acetate, vinyl chloride, vinylidene chloride, oxidants, lactones, lactams, cyclic anhydrides, cyclic siloxanes and combinations thereof. Functionalized versions of these monomers may also be used. Specific monomers or comonomers that may be suitable include, but are not limited to, methyl methacrylate, ethyl methacrylate, propyl methacrylate (all isomers), butyl methacrylate (all isomers), 2-ethylhexyl methacrylate, isobornyl methacrylate, methacrylic acid, benzyl methacrylate, phenyl methacrylate, methacrylonitrile, a-methylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, methacrylamide, N-methylacrylamide, N,N-dimethylacrylamide, N-tert-butylmethacrylamide, N-n-butylmethacrylamide, N-methylolmethacrylamide, N-ethylohnethacrylamide, N-tert-butylacrylamide, N-n-butylacrylamide, N-methylolacrylamide, N-ethylolacrylamide, vinyl benzoic acid (all isomers), diethylaminostyrene (all isomers), a-methylvinyl benzoic acid (all isomers), diethylamino alpha-methylstyrene (all isomers), p-inylbenzene sulfonic acid, p-vinylbenzene sulfonic sodium salt, trimethoxysilylpropyl methacrylate, triethoxysilylpropyl methacrylate, tributoxysilylpropyl methacrylate, dimethoxymethylsilylpropyl methacrylate, diethoxymethylsilylpropyl methacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, diisopropoxysilylpropyl methacrylate, trimethoxysilylpropyl acrylate, triethoxysilylpropyl acrylate, tributoxysilylpropyl acrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, maleic anhydride, N-phenylmaleimide, N-butylmaleimide, chloroprene, ethylene, vinyl acetate, vinyl chloride, vinylidene chloride, 2-(2-oxo-1-imidazolidinyl)ethyl 2-methyl-2-propenoate, 1-[2-[2-hydroxy-3-(2-propyl)propyl]amino]ethyl]-2-imidazolidinone, N-vinyl pyrrolidone, N-vinyl imidazole, crotonic acid, vinyl sulfonic acid, and combinations thereof.

As would be appreciated by the person skilled in the art, the monomers useful in the preparation of the polymers depend on the particular polymerisation method being used. For living/controlled radical polymerisation, for example, the monomers are selected from olefinically unsaturated monomers. These may be any type of unsaturated monomer ranging from low molecular weight monomers, such as vinyl, to large macromers. These monomers include those of formula 1:

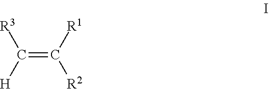

where R1 and R3 are independently selected from the group consisting of hydrogen, halogen, optionally substituted C1-C4 alkyl wherein the substituents are independently selected from the group consisting of hydroxy, —CO2H, —CS2H, —CO2RN, —CS2RN, —CORN, —CSRN, —CSOH, —CSORN, —COSH, —COSRN, —CSOH, —CSORN, —CN, —CONH2, —CONHRN, —CONRN2, —ORN, —SRN, —O2CRN, —S2CRN, —SOCRN, and —OSCRN; and
R2 is selected from the group consisting of hydrogen, RN, —CO2H, —CS2H, —CO2RN, —CS2RN, —CORN, —CSRN, —CSOH, —CSORN, —COSH, —COSRN, —CSOH, —CSORN,
—CN, —CONH2, —CONHRN, —CONRN2, —ORN, —SRN, —O2CRN, —S2CRN, —SOCRN, and —OSCRN;
where RN is selected from the group consisting of optionally substituted C1-C18 alkyl, C2-C18 alkenyl, aryl, heteroaryl, carbocyclyl, heterocyclyl, aralkyl, heteroarylalkyl, alkaryl, alkylheteroaryl, and polymer chains wherein the substituents are independently selected from the group consisting of alkyleneoxidyl (epoxy), hydroxy, alkoxy, acyl, acyloxy, formyl, alkylcarbonyl, carboxy, sulfonic acid, alkoxy- or aryloxy-carbonyl, isocyanato, cyano, silyl, halo, amino, or a substituent of biological origin or activity, such as saccharide, peptide, antibody, nucleic acid or the like;
   including salts, inner salts, such as zwitterions and derivatives thereof.

Examples of monomers include, but are not limited to, maleic anhydride, N-alkylmaleimide, N-arylmaleimide, dialkyl fumarate and cyclopolymerisable monomers, acrylate and methacrylate esters, acrylic and methacrylic acid, styrene, acrylamide, methacrylamide, and methacrylonitrile, mixtures of these monomers, and mixtures of these monomers with other monomers. As one skilled in the art would recognise, the choice of comonomers is determined by their steric and electronic properties. The factors which determine copolymerisability of various monomers are well documented in the art. For example, see: Greenley, R Z. in Polymer Handbook 3rd Edition (Brandup, J., and Immergut, E. H Eds.) Wiley: New York. 1989 pII/53.

Specific examples of monomers or comonomers include the following: methyl methacrylate, ethyl methacrylate, propyl methacrylate (all isomers), butyl methacrylate (all isomers), 2-ethylhexyl methacrylate, isobornyl methacrylate, methacrylic acid, benzyl methacrylate, phenyl methacrylate, methacrylonitrile, alpha-methylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, functional methacrylates, acrylates and styrenes selected from glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, methacrylamide, N-methylacrylamide, N,N-dimethylacrylamide, N-tert-butylmethacrylamide, N-n-butylmethacrylamide, N-methylolmethacrylamide, N-ethylohnethacrylamide, N-tert-butylacrylamide, N-n-butylacrylamide, N-methylolacrylamide, N-ethylolacrylamide, vinyl benzoic acid (all isomers), diethylamino styrene (all isomers), alpha-methylvinyl benzoic acid (all isomers), diethylamino alpha-methylstyrene (all isomers), p-vinylbenzene sulfonic acid, p-vinylbenzene sulfonic sodium salt, trimethylsilyl methacrylate, trimethoxysilylpropyl methacrylate, triethoxysilylpropyl methacrylate, tributoxysilylpropyl methacrylate, dimethoxymethylsilylpropyl methacrylate, diethoxymethylsilylpropyl methacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, diisopropoxysilylpropyl methacrylate, trimethylsilyl acrylate, trimethoxysilylpropyl acrylate, triethoxysilylpropyl acrylate, tributoxysilylpropylacrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, vinyl acetate, vinyl butyrate, vinyl benzoate, vinyl chloride, vinyl fluoride, vinyl bromide, maleic anhydride, N-phenylmaleimide, N-butylmaleimide, N-vinylpyrrolidone, 2,2-dimethyl azlactone, N-vinylcarbazole, butadiene, isoprene, chloroprene, ethylene, propylene, 2-methacryloyloxy ethyl phosphorylcholine, 2-acryloyloxy ethyl phosphorylcholine, 3-methacryloylamino propyl dimethyl-3-sulfopropyl ammonium hydroxide inner salt, 2-methacryloyloxy ethyl dimethyl-3-sulfopropyl ammonium hydroxide inner salt, trimethylsilylethyl methacrylate, ethoxyethyl methacrylate, N-3-NNNN-dicarboxymethyl aminopropyl methacrylamide, tetrahydrofurfuryl methacrylate, glycerol methacrylate, 2-methacryloylethyl glucoside.

In one embodiment, the dendritic molecule of the invention may be functionalised to modify the structure and/or function of the molecule. The dendritic macromolecule may be functionalised by the addition of one or more chemical moieties to the outermost generational polymers of the dendritic molecule, (ie, modification of the dendrimer surface), the addition of one or more chemical moieties to the first and/or further generational polymers, and/or encapsulating one or more small molecules within the cavities within the dendritic molecule.

When a dendritic macromolecule is functionalised by the addition of one or more chemical moieties to the outermost generational polymers of the dendritic molecule and/or the addition of one or more chemical moieties to the first and/or further generational polymers, the chemical moiety may be any moiety suitable for the desired structure or function of the dendritic molecule. For example, suitable chemical moieties include, but are not limited to, ligands for receptors, property modifiers, pharmaceuticals, signaling moieties, genetic material and the like. In this way, dendritic molecules may be produced which exhibit a range of functional properties to enable the dendritic molecule to exhibit strong affinity for, and interact with a target entity, cross the cell wall to transport genetic material into cells, and so on,

Ligands for receptors include, but are not limited to, mono and oligosaccharides or analogues thereof, peptide ligands or fragments or analogues thereof, and small molecules which are receptor agonists or antagonists, or fragments thereof.

Property modifiers include, but are not limited to, solubility modifiers, hydrophilic groups (eg; PEGs or other hydrophilic polymers, polyhydroxyl chains, oligosaccharides, aryl or heteroaryl groups and the like), hydrophobic groups (eg; long chain alkyl groups, steroids, and the like), charged end groups (eg; groups with a negative charge, groups with a positive charge, groups that are zwitterionic).

Pharmaceuticals include any pharmaceutically active component including, but not limited to, one or more selected from the group consisting of analgesics, anti-arthritic, antibiotics, anti-convulsants, anti-fungals, anti-histimines, anti-infectives, anti-inflammatories, anti-microbials, anti-protozoals, antiviral pharmaceuticals, contraceptives, growth promoters, hematinics, hemostatics, hormones and analogues, immunostimulants, minerals, muscle relaxants, vaccines and adjuvants, vitamins or their mixtures thereof. The pharmaceutical may be bound directly to the macromolecule, or may be bound to the macromolecule via a cleavable linker. The cleavable linker may be of any suitable type (eg; acid labile, reductively labile, enzymatically cleavable (eg; protease, esterase and the like)).

Signaling moieties include, but are not limited to, radioactive labels, PET labels, PET active, MRI active, fluorescent labels, and the like. Suitable signaling moieties include, but are not limited to, radio active halogen atoms, lanthanide metal ions (eg, gadolinium ions).

Genetic material includes a DNA sequence or a RNA sequence.

The cavities within dendritic molecules may be used to encapsulate small molecules, including but not limited to, one or more pharmaceutically active components.

For many applications it is desirable that an active molecule be bound to the dendritic molecule. The dendritic molecules of the present invention are particularly advantageous as the active can be bonded at any predetermined site of the dendritic molecule (or dendron). Even more advantageously, more than one active can be bonded to the dendritic molecule of the invention. Where more than one active molecule is bound to the dendritic molecule, the active molecules may be the same or different.

It may be desirable to protect the active molecule(s) to allow delivery of the active molecule(s) to be targeted to particular sites in the body. The purpose of protecting the active molecule may include, but is not limited to, protecting the active molecule from destruction in harsh conditions, targeting delivery of the active molecule to the specific site of action, preventing delivery of the active molecule to healthy regions of the body.

The active molecule may be a pharmaceutical, a chemical entity, a chemotherapy agent, a carbohydrate, a saccharide chain, a radio-isotope for in vivo diagnostic purposes, a peptide, a polypeptide, a protein, a form of DNA, a form of RNA including small interfering RNA (siRNA) and/or or other nucleic acid, such as PNA and/or a molecule that modifies the properties of the dendritic molecule. Mixtures of the above are also envisaged. This is by no means an exhaustive list and it will be appreciated that any active molecule can be bonded or attached to any part of the dendron or dendrimer depending upon the end use and application of the dendron or dendrimer.

One or more active molecules may also be bound to the surface of the dendritic macromolecule to protect the dendritic macromolecule from destruction in harsh conditions. In such a case, the active molecule functions as a coating.

Pharmaceuticals include any pharmaceutically active component including, but not limited to, one or more selected from the group consisting of analgesics, anti-arthritic, antibiotics, anti-angiogenics, anti-cancers, anti-convulsants, anti-fungals, anti-histimines, anti-infectives, anti-inflammatories, anti-microbials, anti-protozoals, antiviral pharmaceuticals, contraceptives, growth promoters, hematinics, hemostatics, hormones and analogues, immunostimulants, minerals, muscle relaxants, vaccines and adjuvants, vitamins and mixtures thereof.

The active molecule may be bound directly to the macromolecule, or may be bound to the macromolecule via a cleavable linker. The cleavable linker may be of any suitable type (eg; biodegradable, acid labile, reductively labile, enzymatically cleavable (e.g.; protease, esterase), degradable (e.g., by heat, UV light, oxidation, reduction) and the like).

Therefore the invention also relates to a delivery molecule comprising a dendron or dendritic molecule and one or more active molecules, wherein the active molecules are bound to the dendritic macromolecule by a degradable or cleavable linkage.

Preferably the dendron or dendritic molecule is according to the invention.

Preferably the linkage is biodegradable.

The biodegradable or cleavable linkage may be of any type known to the person skilled in art. Preferably, the biodegradable or cleavable linkage is selected so as to be degraded or cleaved to release the active molecule at an appropriate time. For example, the biodegradable or cleavable linkage may be selected to enable the delivery of the active molecule to a particular site in the body, or to enable the staggered release of a number of active molecules from the dendritic molecule (whether the same active molecule or different active molecules) in the body.

In preparing the delivery molecule, it is advantageous that any residual functional groups on the dendron or dendrimer be protected or capped by methods known in the art. If required, the polymer arms, in particular the pendant groups can be deprotected or reacted to form a functional group more amenable to bonding to an active. As an example, acrylate end groups can be converted to the corresponding acid groups. Once the active is bound or linked, the dendrimer can be micellised to give an amphiphilic dendrimer molecule.

It will be appreciated from the above discussion that by precise synthesis of the first polymer and the generational polymers and dendrons it is possible to generate a number of hitherto unprecedented dendritic molecules by way of a reasonably small number of iterative reactions. The method of the present invention therefore provides for a hitherto unknown flexibility in forming the dendritic molecule as well as the resulting structure. Further the methods and molecules of the invention retain the advantageous properties of dendritic molecules like narrow polydispersity and controlled architecture.

The invention is now described by way of non-limiting examples and/or drawings.

### EXAMPLES

Reagents

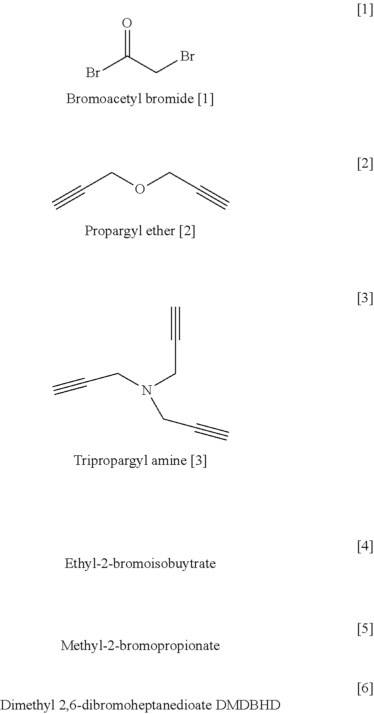

Analytical Methodologies

¹H and ¹³C Nuclear Magnetic Resonance (NMR)

All NMR spectra were recorded on a Broker DRX 500 MHz spectrometer using an external lock (D₂O, CDCl₃) and utilizing a standard internal reference (1,4-dioxane, solvent reference). ¹³C NMR spectra were recorded by decoupling the protons and all chemical shifts are given as positive downfield relative to these internal references.

### Size Exclusion Chromatography (SEC)

The molecular weight distributions of the polymers were measured by SEC. All polymer samples were dried prior to analysis in a vacuum oven for two days at 40° C. The dried polymer was dissolved in tetrahydrofuran (THF) (Labscan, 99%) to a concentration of 1 mg/mL. This solution was then filtered through a 0.45 μm PTFE syringe filter. Analysis of the molecular weight distributions of the polymer nanoparticles was accomplished by using a Waters 2690 Separations Module, fitted with two Ultrastyragel linear columns (7.8×300 mm) kept in series. These columns were held at a constant temperature of 35° C. for all analyses. The columns used separate polymers in the molecular weight range of 500-2 million g/mol with high resolution. THF was the eluent used at a flow rate of 1.0 mL/min. Calibration was carried out using narrow molecular weight PSTY standards (PDI≦1.1) ranging from 500-2 million g/mol. Data acquisition was performed using Waters Millenium software (ver. 3.05.01) and molecular weights were calculated by using a 5^{th} order polynomial calibration curve.

The absolute Mw's of the polymer constructs were determined using a PL-GPC-50 SEC system using dual angle light scattering, UV and RI detection operating in THF. Separation was achieved using two PLgel 5 μm (300*7.5 mm) MIXED C GPC columns held at 35° C.

Attenuated Total Reflectance Fourier Transform Spectroscopy (ATR-FTIR)

ATR-FTIR spectra were recorded between 4000 and 550 cm-1 in a Perkin Elmer FT-2000 FTIR spectrometer equipped with a single reflection diamond window. Each spectrum had a 32 scan accumulation using a spectral resolution of 8 cm-1.

Dynamic Light Scattering (DLS)

Dynamic light scattering measurements were performed using a Malvern Zetasizer Nano Series running DTS software and operating a 4 mW He—Ne laser at 633 nm. Analysis was performed at an angle of 90° and a constant temperature of 25° C. Dilute particle concentrations ensure that multiple scattering and particle-particle interactions can be considered negligible during data analysis. The number average hydrodynamic particle size is reported (Dₕ).

Transmission Electron Microscopy (TEM)-Ambient-TEM

A drop of the micelle solution was allowed to air dry onto a formavar precoated copper TEM support grid. To obtain a negative stain the samples were exposed to a drop of a 2% solution of uranyl acetate for 1 minute after which excess staining solution was removed via careful blotting. The polymer nanoparticles were characterised on a Jeol-1010 instrument utilizing an accelerating voltage of 80 kv operating at ambient temperature.

### Example 1

### Synthesis of Metal-Catalysed Initiators

### Synthesis of 2,2-Dimethyl-1,3-dioxolane-4-methoxy-(2-bromo-2-methylpropionyl) [7]

(reference: Perrier, S.; Armes, S.; Wang, X.; Malet, F.; Haddleton, D., J. Polym. Sci., Part A: Polym. Chem., 2001, 39, 1696-1707)

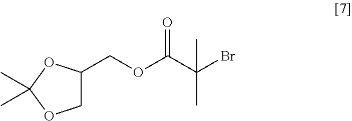

The synthesis of [7] is as follows: DL-1,2-Isopropylideneglycerol (10.63 g, 0.080 mol), triethylamine (9.77 g, 0.097 mol) and THF (50 mL) were added to a round bottom flask and stirred at 0° C. under N₂. A solution of 2-Bromoisobutyryl bromide (23.064 g, 0.10 mol) in THF (100 mL) was added to a pressure equalising funnel, and added drop wise to the reaction vessel over a 1 h period. The reaction mixture was then stirred at room temperature for a further 3 h, after which a white precipitate became visible. The solvent (THF) was removed by rotary evaporation, diethyl ether (50 mL) was added, and the mixture filtered and then washed with a 10% HCl solution (50 mL), brine (50 mL) and Milli-Q water (50 mL). The mixture was then dried over MgSO₄, filtered, the solvent removed by rotary evaporation and dried in vacuo. The product was used without further purification.

¹H NMR (CDCl₃) δ=0.196 (Si(CH₃)₃), 1.350 (3H s, CHCH₂OCCH₂), 1.425 (3H s, CH₂CHOCCH₂), 1.930 (6H s, OC(═O)C(CH₃)2), 3.8-4.3 (5H m)

### Synthesis of 3-hydroxypropyl 2-bromo-2-methylproponoate [8]

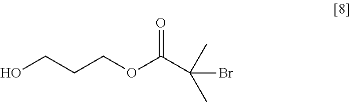

1,3-Propanediol (33.20 g, 0.44 mol) and triethylamine (2.21 g, 0.02 mol) were stirred in THF (60 ml) and cooled in an ice bath. 2-Bromo isobutyrylbromide (5.00 g, 0.02 mol) in THF (40 mL) was added dropwise, and the reaction mixture was stirred overnight at room temperature. The mixture was filtered and the solvent evaporated on a rotary evaporator. The resultant clear oil was re-dissolved in diethyl ether, washed with 10% (v/v) HCl, then with brine and water, and the solvent was then evaporated on a rotary evaporator. The product was purified by column chromatography (with 40/60 ethyl ether/hexane as eluting solvent), resulting in a clear oil. 1H NMR: δ 4.60-4.49 (s, br, 1H, OH); 4.17 (t, ¹J=7.96 Hz, 2H, CH₂); 3.48 (t, ¹J=6.32 Hz, 2H, CH₂); 1.87 (s, 6H, CH₃); 1.75 (q, ¹J=6.32 Hz, 2H, CH₂).

### Synthesis of 3-(1,1,1-trimethylsilyl)-2-propynyl 2-bromo-2-methylpropanoate [9]

(reference: J. A. Opsteen, J. A.; van Hest, J. C. M., Chem. Commun., 2005, 57-59)

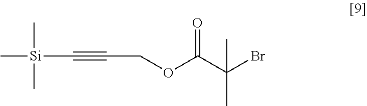

3-(Trimethylsilyl)-2-propyn-1-ol (2.0024 g, 0.0156 mol), triethylamine (2.2858 g, 0.0226 mol), and THF (20 mL) were added to a round bottom flask and stirred at 0° C. under N₂. A solution of 2-Bromoisobutyryl bromide (7.0303 g, 0.0306 mol) in THF (50 la) was added dropwise over 1 h to the reaction mixture. The mixture was stirred at room temperature for 3 h until a white precipitate was visible. Solvent was removed by rotary evaporation, and diethyl ether (50 mL) added. The solution was then filtered and washed with 10% HCl solution (50 mL), brine (50 mL) and Milli-Q water (50 mL), and dried with MgSO₄. The mixture was then filtered, solvent removed by rotary evaporation and dried in vacuo. Purification was achieved with flash column chromatography (distilled hexane/ethyl acetate=19:1). ¹H NMR (CDCl₃) δ=0.164 (Si(CH₃)₃), 1.935 (OC(═O)C(CH₃)₂), 4.745 (CH₂OC(═O)C); ¹³C NMR (CDCl₃) δ=−0.38 (Si(CH₃)₃), 30.64 (OC(═O)C(CH₃)₂), 54.19 (CH₂OC(═O)C), 55.06 (OC(═O)C(CH₃)₂), 92.71 (SiC≡CCH₂OC(═O)), 98.15 (SiC≡CCH₂OC(═O)), 170.81 (OC(═O)C(CH₃)₂). Anal. Calcd. for C₁₀H₁₇: C, 43.32; H, 6.18. Found: C, 43.29; H, 6.25.

### Example 2

### Synthesis of Near Uniform Polymers by Metal-Catalysed ‘Living’ Radical Polymerization

### Synthesis of Polystyrene (PSTY—Br, [10])

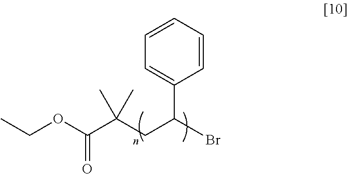

Freshly purified styrene (15.06 g, 0.145 mol), PMDETA (0.190 mL, 9.09×10⁻⁴ mol), ethyl-2-bromoisobutyrate ([4], 0.145 g, 7.44×10⁻⁴ mol) and CuBr₂ (0.0346 g, 1.55×10⁻⁴ mol) was added to a 50 mL round bottom flask then purged with N₂ for 20 min. After 1 h stirring, CuBr (0.109 g, 7.60×10⁻⁴ mol) was added under positive N₂ flow, the flask sealed and purged with N₂ for a further 5 min. The flask was placed in a temperature controlled oil bath at 80° C. for 3 h 25 min. The reaction was terminated by quenching with liquid nitrogen and then exposure to air. The polymerization mixture was diluted with THF then the copper salts removed by passage through an activated basic alumina column. The solution was concentrated by airflow and the polymer recovered by precipitation into methanol, filtration and drying for 48 h under high vacuum at 25° C. The polymer [10] was characterized by SEC (Mₙ=5125, PDI=1.09).

### Synthesis of PMA-Br [11]

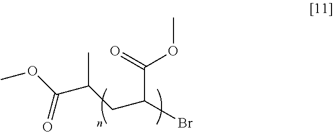

Freshly purified methyl acrylate (25.057 g, 0.291 mol), PMDETA (0.304 g, 1.45×10⁻³ mol) and ethyl-2-bromoisobutyrate ([4], 0.216 g, 1.46×10⁻³ mol) and anisole (10 mL) were added to a 50 mL, Schlenk flask equipped with a magnetic stirrer then purged with N₂ for 15 min. CuBr (0.104 g, 7.28×10⁻⁴ mol) and CuBr₂ (0.162 g, 7.28×10⁻⁴ mol) was then added under positive N₂ flow then the mixture was flushed with N₂ for a further 10 min. The mixture was placed in an oil bath at 50° C. for 24 h. The polymerization was stopped by exposing the reaction mixture to air. The reaction was diluted with chloroform, and the copper salts were removed by passing through a basic alumina column. The polymer solution was washed 3 times with water and the organic layer dried over anhydrous MgSO₄. The polymer then recovered by removal of the chloroform under vacuum. The polymer [11] was dried for 24 h under vacuum at 25° C., and analysed by SEC (Mₙ=7372, PDI=1.06).

### Synthesis of P^{t}BA-Br [12]

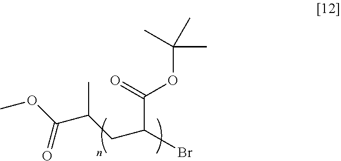

Freshly purified tert-butyl acrylate (15.03 g, 0.117 mol), PMDETA (0.516 mL, 2.47×10⁻³ mol), methyl-2-bromopropionate ([5], 0.392 g, 2,35×10⁻³ mol), CuBr₂ (0.029 g, 1.30×10⁻⁴ mol) and acetone (4.2 mL) were added to a 50 mL round bottom flask, equipped with a magnetic stirrer, and purged with N₂ for 20 min. After 1 h stirring, CuBr (0.338 g, 2.36×10⁻³ mol)was added under positive N₂ flow purged with N₂ for a further 5 min, and then sealed. The flask was placed in a temperature controlled oil bath at 60° C. for 4 h. The reaction was terminated by quenching with liquid nitrogen and exposure to air. The polymerization mixture was diluted with THF then the copper salts removed by passage through an activated basic alumina column. The solution was concentrated by airflow, and the polymer recovered by precipitation into cold 50/50 v/v MeOH/Water. The filtrate was dried for 48 h under high vacuum at 25° C. The polymer [12] was characterized by SEC (Mₙ=6186, PDI=1.10).

### Synthesis of Br—PSTY—Br [13]

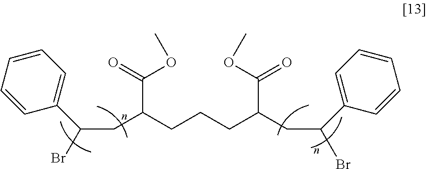

Freshly purified styrene (16.236 g, 0.156 mol), PMDETA (0.332 mL, 1.59×10³ mol), DMDBHD ([6], 0.279 g, 8.1×10⁻⁴ mol) was added to a 50 mL Schlenk flask equipped with a magnetic stirrer. The solution was degassed by 4 freeze-pump-thaw cycles under high vacuum. The Schlenk flask was then flushed with high purity argon and CuBr (0.114 g, 7.9×10⁻⁴ mol) added carefully added under argon flow. The flask was sealed, and polymerization commenced by heating to 100° C. for 20 min. The reaction was terminated by quenching with liquid nitrogen and exposure to air. The polymerization mixture was diluted with THF, and the copper salts removed by passage through an activated basic alumina column. The solution was concentrated by airflow, and the polymer recovered by precipitation into methanol. The filtrate was dried for 48 h under high vacuum at 25° C. The polymer [13] was characterized by SEC (Mₙ=3560, PDI=1.11).

### Synthesis of Sol-PSTY—Br [14]

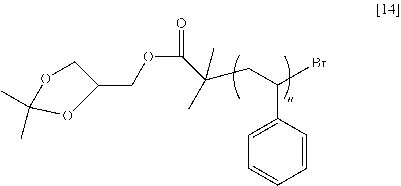

Freshly purified styrene (30.0 g, 0.288 mol), PMDETA (0.262 g, 1.5×10⁻³ mol), [7] (0.427 g, 1.5×10⁻³ mol) and pre-formed CuBr₂/PMDETA complex (0.061 g, 1.5×10⁻⁴ mol) were added to a 50 mL round bottom flask equipped with a magnetic stirrer, and purged with N₂ for 20 min. Under a positive N₂ flow, CuBr (0.216 g, 1.5×10⁻³ mol) was added, the flask sealed and purged with IV, for a further 5 min. The flask was placed in an oil bath at 80° C. for 2 h. The polymerization was stopped by quenching with liquid N₂, dilution with THF and exposure to air. The copper salts were removed by passage through an activated basic alumina column. The polymer [14] was precipitated in MeOH, then filtered and dried for 24 h under high vacuum. The polymer analysed by SEC (Mₙ=4661, PDI=1.09).

### Synthesis of HO—PSTY—Br [15]

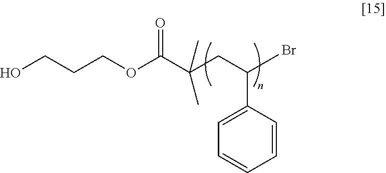

Freshly purified styrene (3.0 g, 2.88×10⁻² mol), PMDETA (0.026 g, 1.5×10⁻⁴ mol), pre-formed CuBr₂/PMDETA complex (0.00595 g, 1.5×10⁻⁵ mol), and [8] (0.031 g, 1.38×10⁻⁴ mol) were added to a 10 mL Schlenk flask equipped with a magnetic stirrer, and the reaction mixture deoxygenated by bubbling with a stream of N₂ for 15 min. CuBr (0.0215 g, 1.5×10⁻⁴ mol) was then added under N₂ and the reaction mixture was further flushed with N₂ for 10 min., and placed in an oil bath at 80° C. for 2 h. The polymerization was stopped by exposing the reaction to air and dilution with DMF (approx. 30 mL). The copper salts were removed by passage through activated basic alumina column. The polymer [15] was precipitated in a large volume of MeOH, filtered and dried for 24 h in vacuo at 40° C. (SEC analysis gave an Mₙ=6258 and PDI=1.10).

### Synthesis of TMS—≡—PSTY—Br [16]

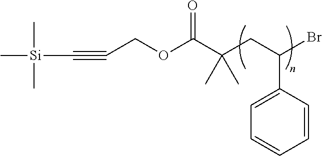

Freshly purified styrene (27.0866 g, 0.26 mol), anisole (5 mL), [9] (0.5996 g, 2.16×10⁻³ mol), CuBr₂/PMDETA complex (0.2104 g, 5.3×10⁻⁴ mol) and PMDETA (0.4503 mL, 2.15×10⁻³ mol) were added to a round bottom flask and degassed by purging with argon. A positive pressure of argon was permitted to flow through the system, and CuBr (0.3727 g, 2.6×10⁻³ mol) was added. A rubber septum was immediately fitted to the flask, the vessel placed in an oil bath at 80° C., and the reaction mixture was stirred, and the polymerization stopped after 2 h. The polymerization was quenched with liquid N₂ and exposed to air. The excess styrene was evaporated off; and THF was added to the reaction mixture. The solvent was removed, and the polymer mixture dissolved in CHCl₃. The solution was washed 3 times with water to remove the copper, dried with MgSO₄, and reduced in volume under N₂ stream, before precipitating into methanol. The polymer [16] was then collected by vacuum filtration and the molecular weight distribution measured by SEC (Mₙ=4651, PDI=1.085).

### Synthesis of TMS—≡—P^{t}BA-Br [17]

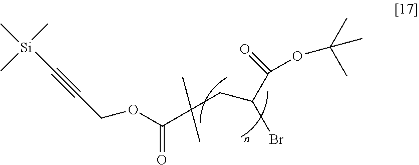

Freshly purified tert-butyl acrylate (8.83 g, 0.07 mol), PMDETA (0.12 g, 7.16×10⁻⁴ mol), CuBr₂ (0.02 g, 6.72×10⁻⁵ mol), [9] (0.32 g, 1.16×10⁻³ mol) and acetone (2.5 mL) were added to a 50 mL Schlenk flask equipped with a magnetic stirrer and purged with N₂ for 15 min. CuBr (0.1 g, 6.81×10⁻⁴ mol) was then added under positive N₂ flow then the mixture was further flushed with N₂ for 10 min. The mixture was placed in an oil bath at 60° C. for 220 min. The polymerization was stopped by exposing the reaction to air. The reaction medium was diluted with chloroform and the copper salts were removed by extraction with water. The organic layer was dried with anhydrous MgSO₄ and the polymer then recovered by removal of the chloroform under vacuum. The polymer [17] was dried for 24 h under vacuum at 25° C. (Mₙ=4200, PDI=1.11).

### Synthesis of TMS—≡—PMA-Br [18]

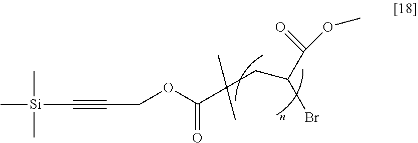

Freshly purified methyl acrylate (3.824 g, 0.044 mol), PMDETA (0.048 g, 2.78×10⁻⁴ mol), CuBr₂/PMEDTA complex (0.0056 g, 1.41×10⁻⁵ mol), 191 (0.154 g, 5.6×10⁻⁴ mol) and anisole (1.6 mL) were added to a 20 mL Schlenk flask equipped was purged with N₂ for 15 min. CuBr (0.0398 g, 2.78×10⁻⁴ mol) was then added under positive N₂ flow, and the mixture further flushed with N₂ for 10 min. The mixture was placed in an oil bath at 50° C. for 220 min. The polymerization was stopped by exposing the reaction to air. The reaction medium was diluted with chloroform, and the copper salts were removed by extraction with water. The organic layer was dried over anhydrous MgSO₄, and the polymer recovered by removal of the chloroform under vacuum. The polymer [18] was dried for 24 h under vacuum at 25° C. (SEC: Mₙ=5339, PDI=1.09).

### Example 3

### Azidation of ATRP Polymers

### Synthesis of PSTY—N₃ [19]

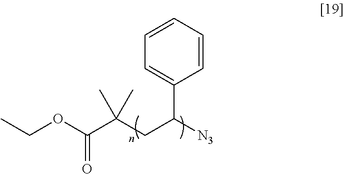

A typical azidation procedure was as follows: PSTY—Br ([10], 2.0 g, 0.39 mmol) was dissolved in 20 mL of DMF in a 50 mL screw-capped vial. NaN₃ (0.278 g, 4.3 mmol) was added, and the mixture stirred for 24 h at 50° C. The polymer was precipitated in MeOH, recovered by vacuum filtration and washed exhaustively with water and MeOH. The polymer [19] was dried under vacuum for 48 h at 25° C.

PMA-Br [11] and P^{t}BA-Br [12] were azidated using the same procedure as above but purified by precipitation into cold 50/50 MeOH/Water, filtered and dried under vacuum to give azidated polymers PMA-N₃ (POD and P^{t}BA-N₃ ([21]).

Structures of [20], [21], [22], [23], and [24].

### PMA-N₃ [20]

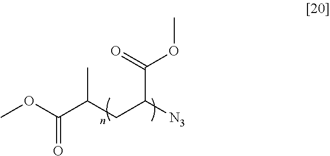

### P^{t}BA-N₃ [21]

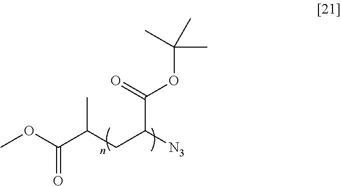

### N₃—PSTY—N₃ [22]

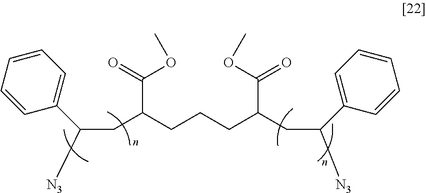

### Sol-PSTY—N₃ [23]

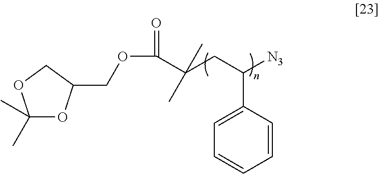

### HO—PSTY—N₃ [24]

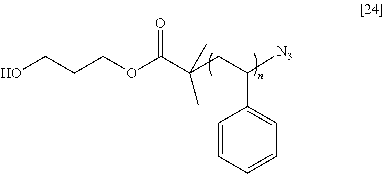

### Synthesis of TMS—≡—P(STY)—N₃ [25]

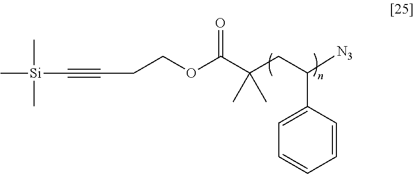

TMS—≡—P(STY)—Br ([16], 2.0 g, 4.00×10⁻⁴ mol) was dissolved in DMF (15 mL). NaN₃ (0.109 mg, 8.68×10⁻⁴ mol) was added and the mixture stirred for 24 h at room temperature. The polymer was precipitated in MeOH, then recovered by vacuum filtration and washed exhaustively with water and MeOH. The polymer [25] was dried for 48 h under vacuum at 25° C.

TMS—≡—P(tBA)-Br [17] and TMS—≡—P(MA)-Br [18] were azidated using the same procedure as above but purified by dilution into chloroform (100 mL) and washing three times with water (100 mL). The chloroform was dried over anhydrous MgSO₄ after which the chloroform was removed under vacuum and the polymer dried for 24 h at 25° C. under vacuum to give the azidated polymers, TMS—≡—P(tBA)-N₃ [26] and TMS—≡—P(MA)-N₃ [27].

### TMS—≡—P(tBA)-N₃ [26]

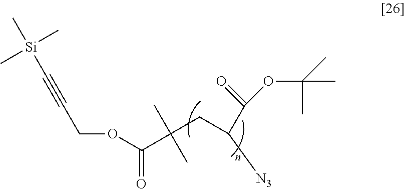

### TMS—≡—P(MA)-N₃ [27]

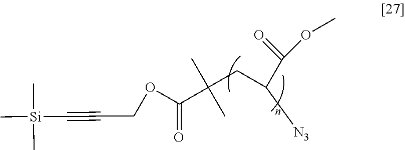

A summary of Examples 1-3 is provided in the following table:

| ATRP Polymer | After Azidation |
|---|---|
| PSTY—Br [10] | PSTY—N₃ [19] |
| PMA—Br [11] | PMA—N₃ [20] |
| P^{t}BA—Br [12] | P^{t}BA—N₃ [21] |
| Br—PSTY—Br [13] | N₃—PSTY—N₃ [22] |
| Sol-PSTY—Br [14] | Sol-PSTY—N₃ [23] |
| HO—PSTY—Br [15] | HO—PSTY—N₃ [24] |
| TMS—≡—PSTY—Br [16] | TMS—≡—PSTY—N₃ [25] |
| TMS—≡—P^{t}BA—Br [17] | TMS—≡—P^{t}BA—N₃ [26] |
| TMS—≡—PMA—Br [18] | TMS—≡—PMA—N₃ [27] |

### Example 4

### Functionalisation of Azidated ATRP Polymers

### Synthesis of Dendron starting core PSTY-(—≡)₂ [28]

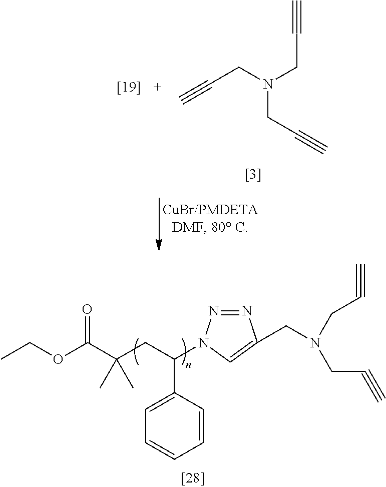

PSTY—N₃ ([19], 0.179 g, 3.49×10⁻⁵ mol), PMDETA (0.075 mL, 3.59×10⁻⁴ mol) and tripropargylamine [3] (0.100 mL, 7.07×10⁻⁴ mol) in DMF (1.8 mL) were added to a 10 mL Schlenk flask, and purged with N₂ for 10 min. CuBr (0.0521 g, 3.63×10⁻⁴ mol) was added under a positive flow of N₂, the flask sealed and purged with N₂ for a further 5 min. The flask was placed in a temperature controlled oil bath at 80° C. for 2 h. The reaction was diluted with 5 mL THF, and passed through activated basic alumina to remove the copper salts. The polymer [28] was precipitated in MeOH, then filtered and dried for 24 h under vacuum.

### Synthesis of symmetrical starting core (≡—)₂—PSTY-(—≡)₂ [29]

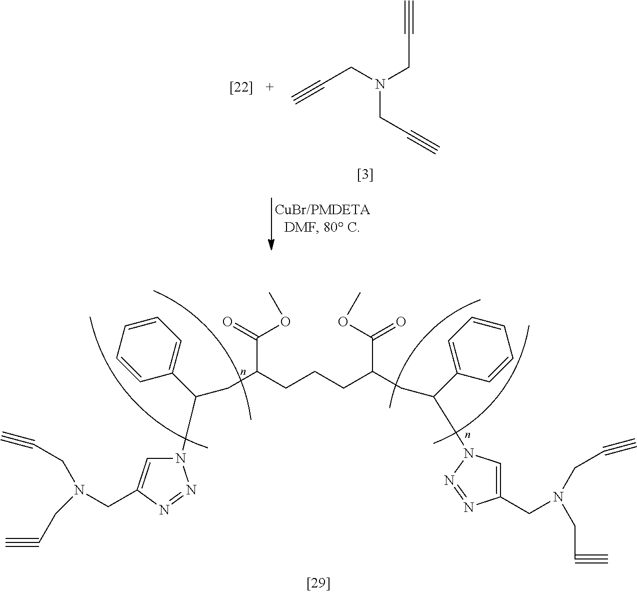

N₃—PSTY₃₄—N₃ ([22], 0.5 g, 1.40×10⁻⁴ mol), PMDETA (0.587 mL, 2.81×10⁻³ mol) and tripropargylamine ([3], 0.791 mL, 5.60×10⁻³ mol) in DMF (5 mL) was added to a 10 mL Schlenk flask, equipped with magnetic stirrer, and purged with N₂ for 10 min. CuBr (0.403 g, 2.81×10⁻³ mol) was added under a positive flow of N₂. the flask was sealed and purged with N₂ for a further 5 min. The flask was placed in a temperature controlled oil bath set at 80° C. for a period of 2 h. The reaction was diluted with 5 mL THF, and passed through activated basic alumina to remove the copper salts. The polymer [26] was precipitated in MeOH, filtered and dried for 24 h under vacuum.

### Synthesis of Sol-PSTY-(—≡) [30]

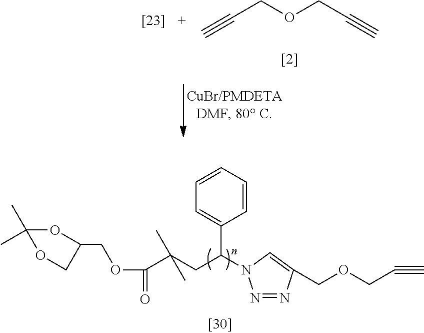

Sol-PSTY—N₃ ([23], 0.501 g, 9.7×10⁻⁵ mol), propargyl ether ([2], 0.210 mL, 2.04×10⁻³ mol), PMDETA (0.035 mL, 1.67×10⁻⁴ mol) in DMF (5 mL) were added to a 10 mL Schlenk flask, and purged with N₂ for 10 min. CuBr (0.0218 g, 1.52×10⁻⁴ mol) was added under a positive flow of N₂, the flask was sealed and purged with N₂ for a further 5 min. The flask was placed in a temperature controlled oil bath set at 80° C. for 2 h. The reaction was diluted with THF, and the copper salts were removed by passage through an activated basic alumina column. The polymer [30] was precipitated in MeOH, filtered and dried under vacuum at 25° C.

### Synthesis of the Propargyl Ether of Dimethyl 5-Hydroxyisophthalate [31]

(Ref: Joralemon, Maisie J.; O'Reilly, Rachel K.; Matson, John B.; Nugent, Anne K.; Hawker, Craig J.; Wooley, Karen L. Macromolecules (2005), 38(13), 5436-5443)

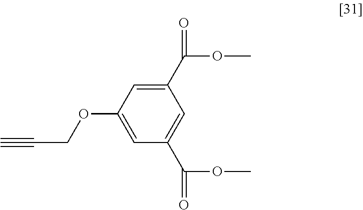

A two-neck 1 L round-bottom flask was charged with dimethyl 5-hydroxyisophthalate (10.0 g, 47.6×10⁻³ mol), acetone (200 mL), K₂CO₃ (7.9 g, 57×10⁻³ mol), 18-crown-6 (0.13 g, 4.9×10⁻⁴ mol), and propargyl bromide (80 wt %) in xylene (6.3 mL, 57×10⁻³ mol). The reaction was reflux overnight under N₂ with stirring. Upon cooling to room temperature, the reaction mixture was filtered and the filter cake was washed with 50 mL of acetone. The filtrate was concentrated by rotary evaporation. The residue was recrystallised in ethanol and dried under vacuum. Isolated yield of [31] (10.45 g, 89%). ¹H NMR (300 MHz, CDCl3): \ddot{α} 2.56 (t, J) 2 Hz, 1H, CH2CtCH), 3.95 (s, 6H, COOCH3), 4.79 (d, J) 2 Hz, 2H, CH2Ct CH), 7.84 (d, J) 2 Hz, 2H, ArH), 8.34 ppm (t, J) 2 Hz, 1H, ArH).

### Synthesis of 1-Propargylbenzene-3,5-dimethanol [32]

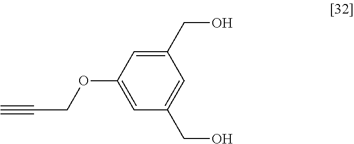

Prior to use, all glassware and the magnetic stir bar were dried in an oven (110° C.) for 1 h. A solution of [31] (10.10 g, 0.04069 mol) in THF (100 mL) was added dropwise to a cold slurry of LiAlH₄ (5.80 g, 0.153 mol) in THF (400 mL) in a flame-dried two-neck 1 L round-bottom flask in an ice bath. The reaction mixture was refluxed with stirring under N₂ for 18 h. A saturated aqueous solution of NH₄OH was added until no more H₂ gas was observed, and then diluted with aqueous HCl (10%) until the pH reached 7. The reaction mixture was filtered, the filter cake was washed with THF, and the filtrate was concentrated by rotary evaporation. The resulting solid was recrystallised in EtOAc:hexane (1/1). Isolated yield of [32] was 5.82 g (75%). ¹H NMR (300 MHz, CD₃OD): δ 2.92 (t, J=2 Hz, 1H, CH₂C≡CH), 4.58 (s, 4H, CH₂OH), 4.73 (d, J=2 Hz, 21-1, CH₂C≡CH), 6.89 (d, J=2 Hz, 2H, ArH), 6.96 ppm (t, J=2 Hz, 1H, ArH). Anal. Calcd. for C₁₁H_{g}: C, 68.74; H, 6.29. Found: C, 68.25; H, 6.21.

### Synthesis of TMS—≡—PSTY—(OH)₂ [33]

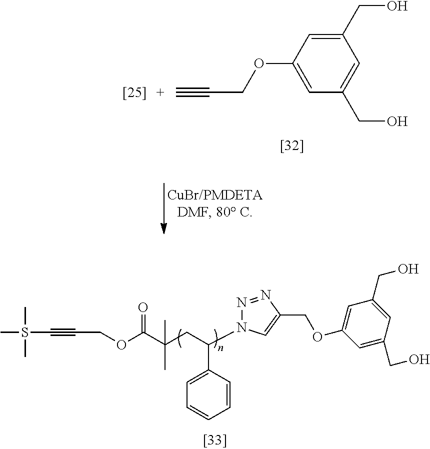

TMS—≡—PSTY—N₃ ([25], 1.0 g 2.00×10⁻⁴ mol), PMDETA (0.035 g, 2.00×10⁻⁴ mol) and [32] (0.156 g, 8×10⁻⁴ mol) in DMF (5 mL) was added to a 10 mL Schlenk flask equipped with a magnetic stirrer. The solution was purged with nitrogen for 10 min. CuBr (0.0286 g, 2.00×10⁻⁴ mol) was then added under positive N₂ flow, and the mixture further flushed with N₂ for 10 min. The mixture was stirred in a temperature controlled oil bath at 80° C. for 60 min. The flask was opened and the solution diluted with chloroform and extracted three times with water. The solution was concentrated under airflow, and the polymer precipitated into methanol, recovered by filtration and washed with MeOH. The polymer [33] was dried for 48 h under vacuum at 25° C.

### Synthesis of TMS—≡—P(tBA)—(OH)₂ [34]

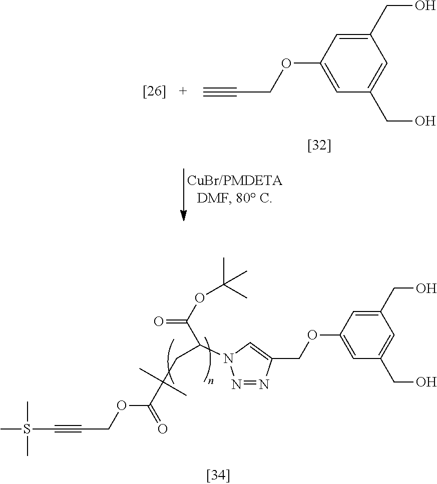

TMS—≡—P(tBA)-N₃ ([26], 1.0 g, 2.34×10⁻⁴ mol), PMDETA (0.021 g, 1.2×10⁻⁴ mol) and [32] (0.182 g, 9.36×10⁻⁴ mol) in DMF (5 mL) were added to a 10 mL Schlenk flask equipped with a magnetic stirrer. The solution was purged with nitrogen for 10 min. CuBr (0.0172 g, 1.2×10⁻⁴ mol) was added under positive N₂ flow, and the mixture further flushed with N₂ for 10 min. The mixture was stirred in a temperature controlled oil bath at 80° C. for 60 min. The flask was opened and the solution diluted with chloroform and extracted 3 times with water. The solution was concentrated under airflow, and the polymer was precipitated into methanol, recovered by filtration and washed with MeOH. The polymer [34] was dried for 48 h under vacuum at 25° C.

### Synthesis of TMS—≡—P(MA)-(OH)₂ [35]

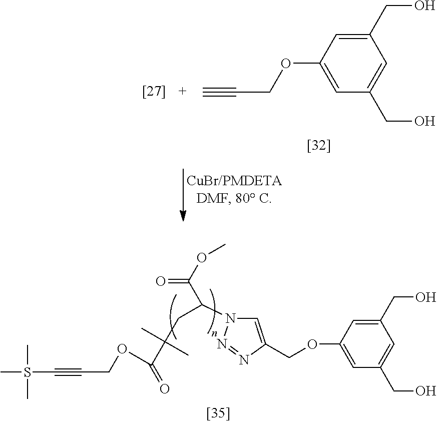

TMS—≡—P(MA)-N₃ ([27], 1.0 g, 1.87×0⁻⁴ mol), PMDETA (0.0162 g, 0.94×10⁻⁴ mol) and [32] (0.145 g, 7.48×10⁻⁴ mol) in DMF (5 mL) were added to a 10 mL Schlenk flask equipped with a magnetic stirrer. The solution was purged with nitrogen for 10 min. CuBr (0.0133 g, 0.94×10⁻⁴ mol) was then added under positive N₂ flow, and the mixture further flushed with N₂ for 10 nun. The mixture was stirred in a temperature controlled oil bath at 80° C. for 60 min. The flask was opened and the solution diluted with chloroform and extracted 3 times with water. The solution was concentrated under airflow and the polymer was precipitated into methanol, recovered by filtration and washed with MeOH. The polymer [35] was dried for 48 h under vacuum at 25° C.

### Synthesis of ≡—P(STY)—(OH)₂ [36]

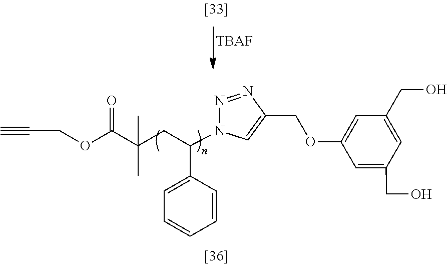

TMS—≡—P(STY)—(OH)₂ ([33], 0.5 g, 9.03×10⁻⁵ mol) was dissolved into THF (5 mL). Tetrabutyl ammonium fluoride hydrate (TBAF, 0.236 g, 9.03×10⁻⁴ mol) was added, and the solution was stirred overnight at 25° C. The polymer [36] was recovered by precipitation into MeOH and dried for 24 h under vacuum at 25° C.

### Synthesis of ≡—P(tBA)-(OH)₂ [37]

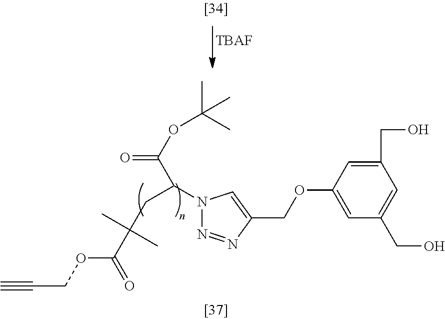

TMS—≡—P(tBA)-(OH)₂ ([34], 0.5 g, 1.19×10⁻⁴ mol) was dissolved in THE (5 mL). TBAF (0.236 g, 9.03×10⁻⁴ mol) was added and the solution was stirred overnight at 25° C. The polymer solution was then taken to dryness under a stream of N₂. The residue was taken up into chloroform (100 mL) and washed 3 times with water (100 mL). The chloroform was removed under vacuum and the polymer [37] dried for 24 h at 25° C. under vacuum.

### Synthesis of ≡—P(MA)-(OH)2 [38]

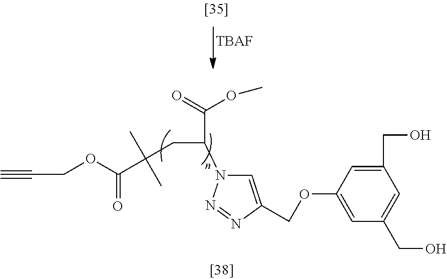

TMS—≡—P(MA)-(OH)₂ ([35], 0.5 g, 9.38×10⁻⁵ mol) was dissolved into THF (5 mL). TBAF (0.236 g, 9.03×10⁻⁴ mol) was added and the solution was stirred overnight at 25° C. The polymer solution was then taken to dryness under a stream of N₂. The residue was taken up into chloroform (100 ML) and washed 3 times with water (100 mL). The chloroform was removed under vacuum and the polymer [38] dried for 24 h at 25° C. under vacuum.

### Example 5

### Synthesis of Reactive (Alkyne or Azide Functional) Beads

### Synthesis of 4-vinylbenzene chloride Crosslinked Beads [39]

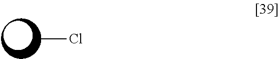

4-vinylbenzene chloride (4 mL, 0.028 mol), styrene (3.2 mL, 0.028 mol), divinylbenzene (3.96 mL, 0.028 mol) and AIBN (6.9 mg, 4.19×10⁻⁵ mol) were added to a 20 mL glass vial equipped with a magnetic stirrer and sealed with rubber septa. The mixture was purged with N₂ for 10 min then heated in a temperature controlled oil bath set at 50° C. for 24 h. The crosslinked polymer was ground to a fine powder with mortar and pestle then stirred in DMF (50 mL) at 50° C. for 1 h. The mixture was filtered hot, and this washing procedure repeated twice. The polymer was then filtered and washed with DMF and then acetone. The polymer [39] was then dried under high vacuum for 16 h.

### Synthesis of propargyl Functionalized Crosslinked Beads [40]

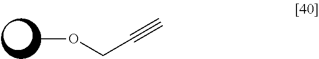

Propargyl alcohol (4.9 mL, 0.087 mol), NaOH (0.07 g, 0.017 mol) and DMF (40 mL) were added to a 50 mL round bottom flask under N₂. The mixture was heated in a temperature controlled oil bath at 40° C. After 20 min, [39] (4 g) was added and the mixture and allowed to stir for 24 h. The mixture was filtered, washed 3 times with water (3×20 mL) and then once with acetone (20 mL). The polymer was then stirred in DMF (50 mL) at 90° C. After 30 min, the mixture was filtered hot and this washing procedure repeated twice, The polymer was then filtered and washed with DMF and then acetone. The polymer [40] was then dried under high vacuum for 16 h.

### Synthesis of azide Functionalized Crosslinked Beads [41]

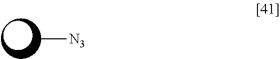

[39] (4 g), NaN₃ (5.68 g, 0.087 mol) and DMF (40 mL) were added to a 50 mL round bottom flask equipped with a magnetic stirrer. The mixture was heated in a temperature controlled oil bath at 50° C. for 48 h. The mixture was filtered and washed 3 times with water (3×20 mL) and once with acetone (20 mL). The functionalised crosslinked polymer was then stirred in DMF (50 mL) at 90° C. After 30 min, the mixture was filtered hot and this washing procedure repeated twice. The polymer was then filtered and washed with DMF and then acetone. The polymer [41] was then dried under high vacuum for 16 h.

### Example 6

### Synthesis of Dendrons

In the nomenclature of dendritic molecules like dendrons and dendrimers the core is termed “generation 0”. Subsequent layers are termed generation 1, 2, 3 and so on. In the present invention, the first polymer is termed generation 0 or G₀. The subsequent generational polymers are termed generation 1, 2, 3 i.e. G₁, G₂ and so on.

Synthesis of Dendron-G₀-G₁-PSTY-Sol [42]

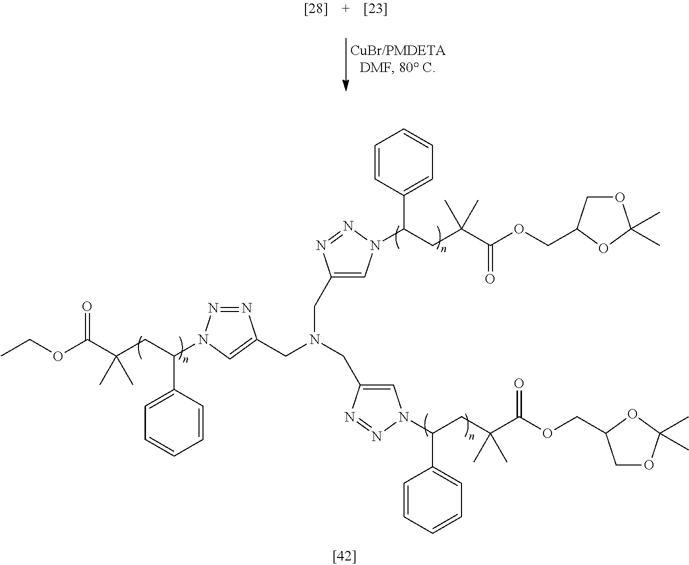

PSTY-(—≡)₂ ([28], 0.110 g, 2.15×10⁻⁵ mol), Sol-PSTY—N₃ ([23], 0.226 g, 4.76×10⁻⁵ mol), PMDETA (0.014 mL, 6.70×10⁻⁵ mol) in DMF (3.5 mL) were added to a 10 mL Schlenk flask, and purged with N₂ for 10 min. CuBr (0.0104 g, 7.3×10⁻⁵ mol) was added under a positive flow of N₂, the flask sealed and purged with N₂ for a further 5 min. The flask was placed in a temperature controlled oil bath at 80° C. for 2 h. The reaction was diluted with 5 mL of THF then passed through activated basic alumina to remove the copper salts.

Further purification of starting material with azide groups from [42] was as follows: THF was removed by evaporation and the polymer [42] in residual DMF was added to a 10 mL Schlenk flask equipped with magnetic stirrer. PMDETA (0.023 mL, 1.1×10⁻⁴ mol) and [40] (0.1 g) were added, and the mixture purged with N₂ for 10 min. CuBr (1.6 mg, 1.11×10⁻⁵ mol) was added under a positive flow of N₂, the flask was sealed and purged with N₂ for a further 5 min. The flask was placed in a temperature controlled oil bath at 80° C. for 4 h. The reaction was filtered hot through a fine glass frit and the beads [40] washed with THF (10 mL). The filtrate was passed through activated basic alumina to remove the copper salts, and the polymer [42]* was precipitated in MeOH, filtered and dried for 24 h under vacuum.

### Synthesis of Dendron-G₀-G₁-PSTY—OH [43]

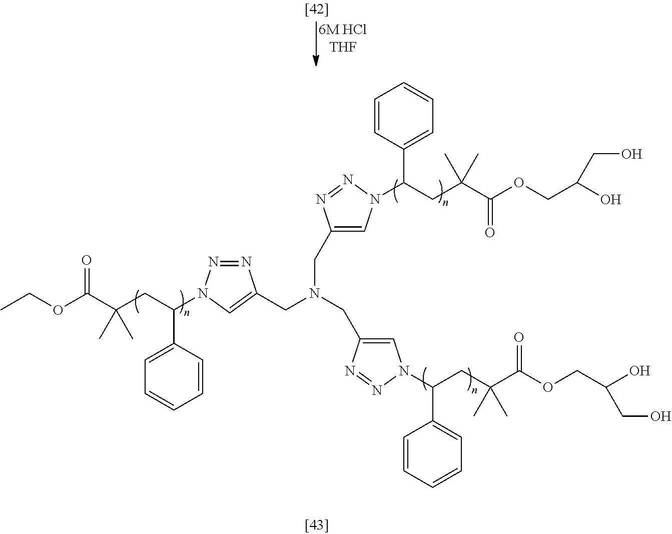

[42] (0.3 g, 2.1×10⁻⁵ mol) was dissolved in 20 mL THF in a 100 mL conical flask equipped with magnetic stirrer. 6M HCl (1 mL) was added dropwise to the solution over 5 min, maintaining the solubility of the polymer. The mixture was allowed to stir for 6 h at room temperature. The polymer [43] was precipitated in MeOH, filtered and dried for 24 h under vacuum.

### Synthesis of Dendron-G₀-G₁-PSTY—Br [44]

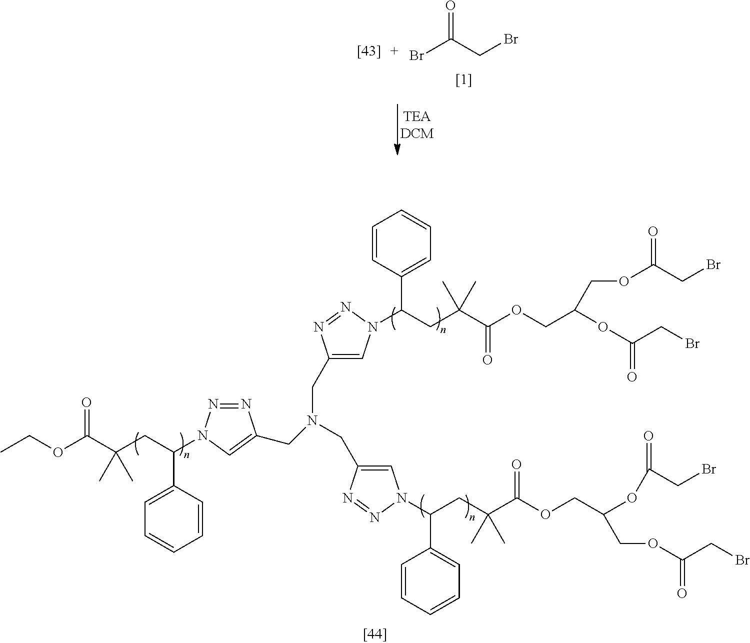

[43] (0.27 g, 1.8×10⁻⁵ mol), triethylamine (0.011 mL, 8.6×10⁻⁵ mol) in 3 mL of dry DCM was added to a 10 mL Schlenk flask equipped with stirrer bar, under N₂. Bromoacetyl bromide ([1], 0.032 mL, 3.67×10⁻⁴ mol) in 2 mL dry DCM was added dropwise to the stirred mixture over 10 min at room temperature. After complete addition, the mixture was allowed to stir for 16 h. The polymer was precipitated in MeOH, filtered and washed 3 times with MeOH (20 mL). The recovered polymer [44] was dried for 24 h under vacuum.

### Synthesis of Dendron-G₀-G₁-PSTY—N₃ [45]

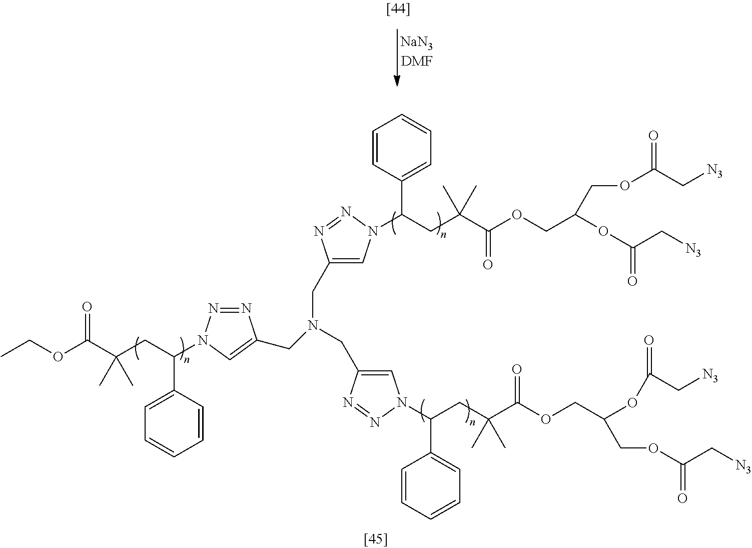

[44] (0.206 g, 1.4×10⁻⁵ mol) was dissolved in 2 mL of DMF. NaN₃ (0.038 g, 5.8×10⁻⁴ mol) was added and stirred for 24 h in a temperature controlled oil bath at 50° C. The polymer [45] was precipitated in MeOH, filtered and dried under vacuum.

### Synthesis of Dendron-G₀-G₁-G₂-PSTY-Sol [46]

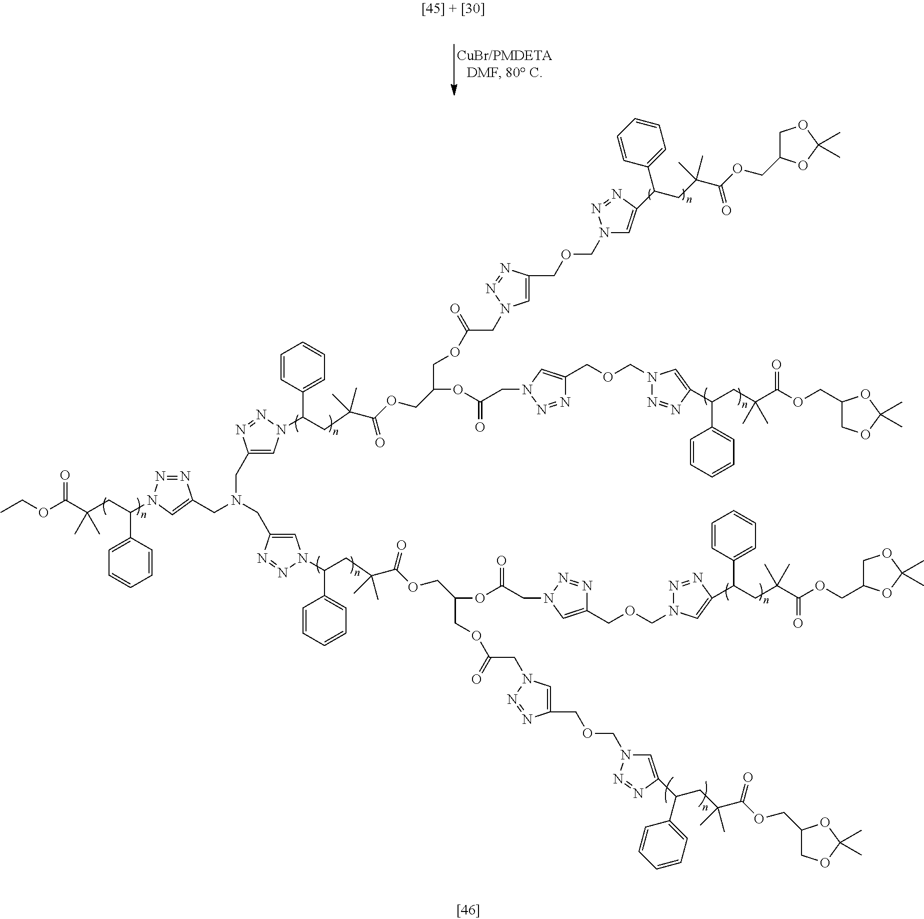

[45] (5.0 mg, 3.4×10⁻⁷ mol), [30] (8.2 mg, 1.5×10⁻⁶ mol), PMDETA (2.9 μL, 1.4×10⁻⁵ mol) in 0.5 mL of DMF were added to a 10 mL Schlenk flask, and purged with N₂ for 10 min. CuBr (2.0 mg, 1.4×10⁻⁵ mol) was added under a positive flow of N₂, the flask was sealed and purged with N₂ for a further 5 min. The flask was placed in a temperature controlled oil bath at 80° C. for a period of 2 h. The reaction was diluted with 5 mL of THF then passed through activated basic alumina to remove the copper salts to give dendron [46].

This procedure was repeated for the synthesis of Dendron-G₀-G₁-G₂-PSTY—(OH)₂ [47] and Dendron-G₀-G₁-PSTY-G₂-P^{t}BA-(OH)₂ [48] using ≡—P(STY)—(OH)₂ [36] and ≡—(^{t}BA)-(OH)₂ [37] respectively.

### Synthesis of Dendron-G₀-G₁-G₂-PSTY—(OH), [47]

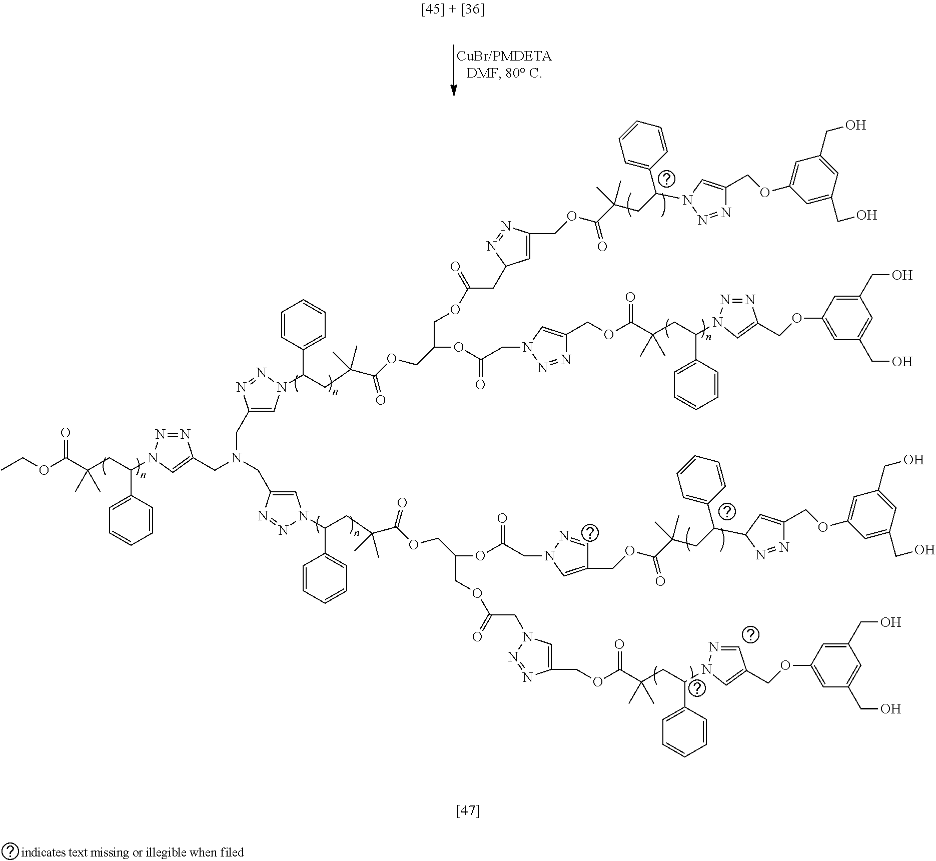

### Synthesis of Dendron-G₀-G₁-PSTY-G₂-P^{t}BA-(OH)₂ [48]

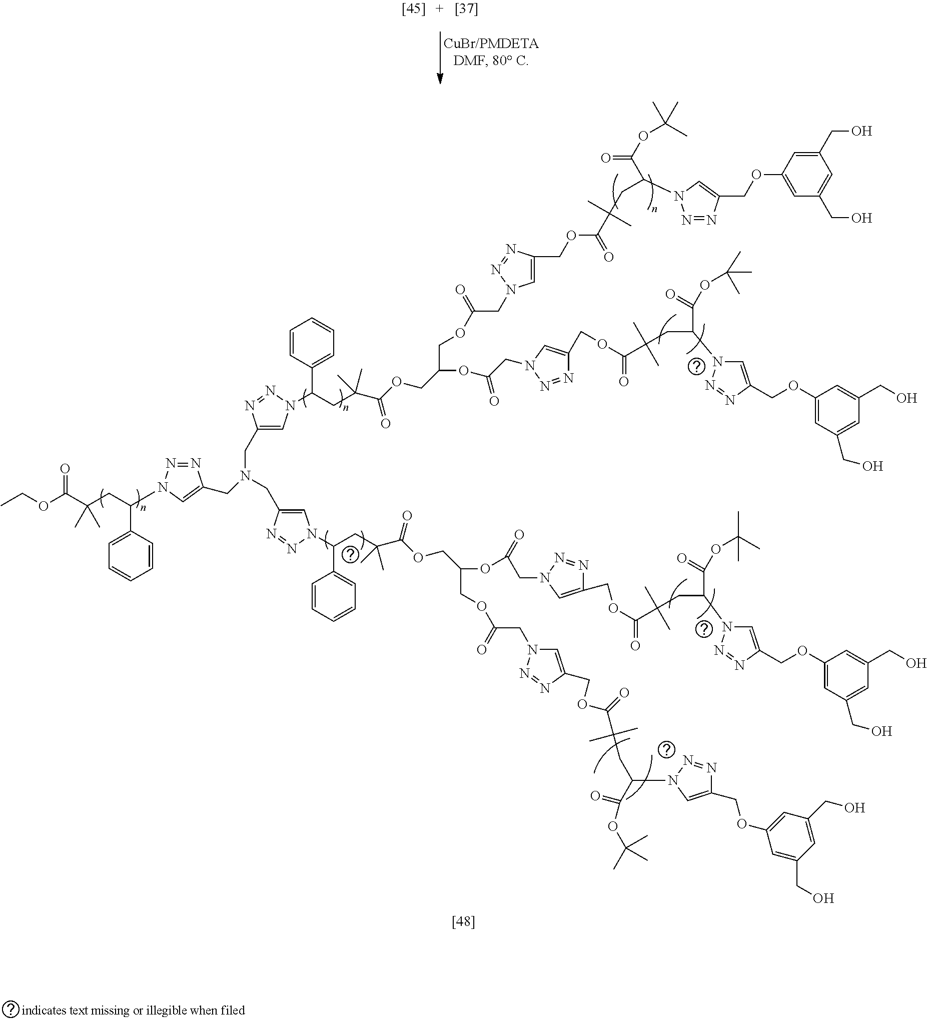

### Degradation of Dendron-G_{D}-G₁-G₂-PSTY-Sol [46]

Where required, the dendrons of Example 6, in particular [46], can be degraded to obtain the constituent arms as follows:

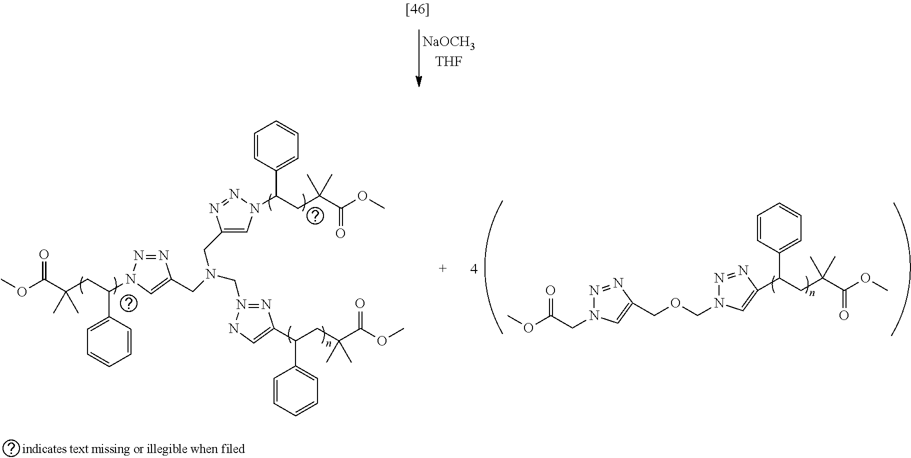

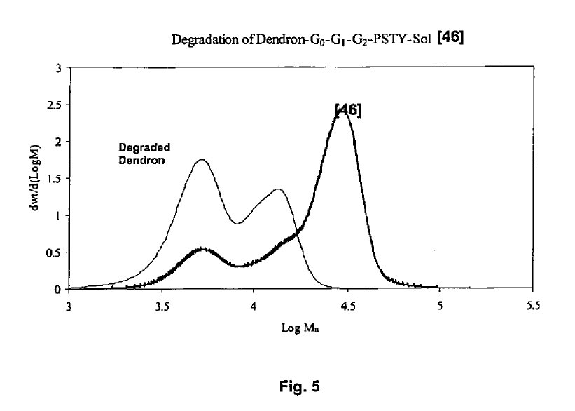

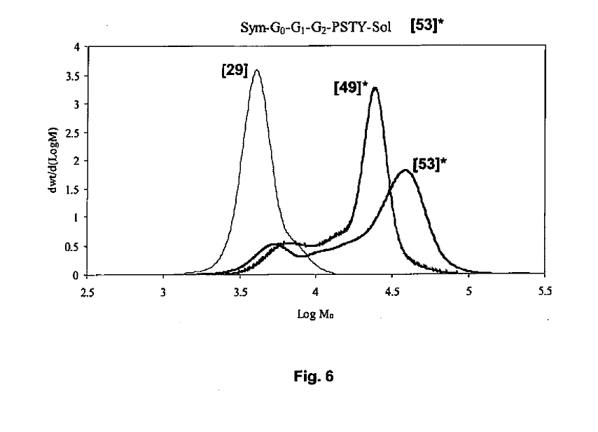

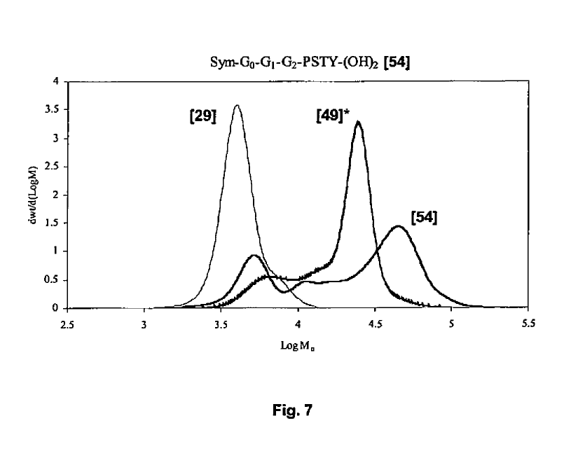

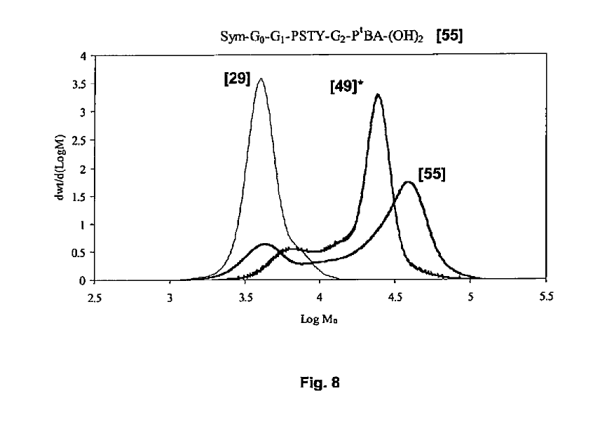

To a 250 μL aliquot of the reaction mixture from the synthesis of Dendron-G₀-G₁-G₂-PSTY-Sol [46] was added THF (1 mL) and NaOCH₃ (10 mg, 1.85×10⁻⁴ mol). The mixture was stirred at room temperature for 16 h, then diluted and analysed by SEC. The SEC is shown in FIG. 5.

The number average molecular weight (Mₙ), polydispersity index (PDI) and the yield of the dendrons of Example 6 are presented in tabular form overleaf.

Size Exclusion Chromatography Analyses

| Starting Functional Polymers/Dendrons | | Dendrons | Mₙ | PDI | Yield % |
|---|---|---|---|---|---|
| PSTY—(—≡)₂ [28] | Sol-PSTY—N₃ [23] | Dendron-G₀-G₁-PSTY-Sol [42] | 14039 | 1.05 | 61 |
| (Mₙ = 5120, PDI = 1.09) | (Mₙ = 4650, PDI = 1.09) | Dendron-G₀-G₁-PSTY-Sol [42]* | 13941 | 1.05 | 85 |
| Dendron-G₀-G₁-PSTY—N₃ [45] | Sol-PSTY—(—≡) [30] | Dendron-G₀-G₁-G₂-PSTY-Sol [46] | 27317 | 1.07 | 68 |
| (Mₙ = 13941, PDI = 1.05) | (Mₙ = 4650, PDI = 1.09) | | | | |
| Dendron-G₀-G₁-PSTY—N₃ [45] | ≡—PSTY—OH)₂ [36] | Dendron-G₀-G₁-G₂-PSTY—(OH)₂ [47] | 29036 | 1.10 | 72 |
| (Mₙ = 13941, PDI = 1.05) | (Mₙ = 4650, PDI = 1.09) | | | | |
| Dendron-G₀-G₁-PSTY—N₃ [45] | ≡—P^{t}BA—(OH)₂ [37] | Dendron-G₀-G₁-PSTY-G₂-P^{t}BA—(OH)₂ [48] | 23722 | 1.08 | 68 |
| (Mₙ = 13941, PDI = 1.05) | (Mₙ = 4200, PDI = 1.11) | | | | |

### Example 7

### Symmetrical Dendrimers

### Synthesis of Sym-G₀-G₁-PSTY-Sol [49]

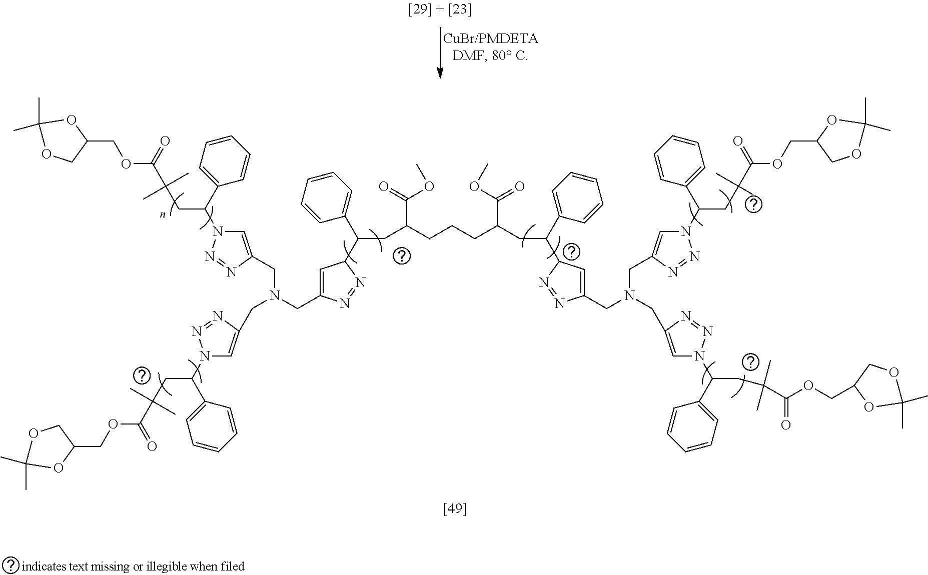

(≡—)₂-PSTY-(—≡)₂ ([29], 0.1 g, 2.5×10⁻⁵ mol), Sol-PSTY—N₃ ([23], 0.512 g, 1.1×10⁻⁴ mol), PMDETA (0.209 mL, 1.0×10⁻³ mol) in 5 mL of DMF were added to a 10 mL Schlenk flask, and purged with N₂ for 10 min. CuBr (0.148 mg, 1.03×10⁻³ mol) was added under a positive flow of N₂, the flask was sealed and purged with N₂ for a further 5 min. The flask was placed in a temperature controlled oil bath at 80° C. for 2 h. The reaction was diluted with 5 mL of THF, and passed through activated basic alumina to remove the copper salts to give dendron [49].

Further purification of starting material with azide groups from [49] was as follows: THF was removed by evaporation and the polymer in residual DMF was added to a 10 mL Schlenk flask, equipped with magnetic stirrer. PMDETA (0.057 mL, 0.27×10⁻⁴ mol) and [40] (0.18 g) were added to the flask and the mixture purged with N₂ for 10 min. CuBr (0.036 g, 2.5×10⁻⁴ mol) was added under a positive flow of N₂, the flask was then sealed and purged with N₂ for a further 5 min. The flask was placed in a temperature controlled oil bath set at 80° C. for 4 h. The reaction was filtered hot through a fine glass frit and the beads washed with THF (10 mL) at the filter. The filtrate was passed through activated basic alumina to remove the copper salts and the polymer [49]* was precipitated in MeOH, then filtered and dried for 24 h under vacuum.

### Synthesis of Sym-G₀-G₁-PSTY—OH [50]

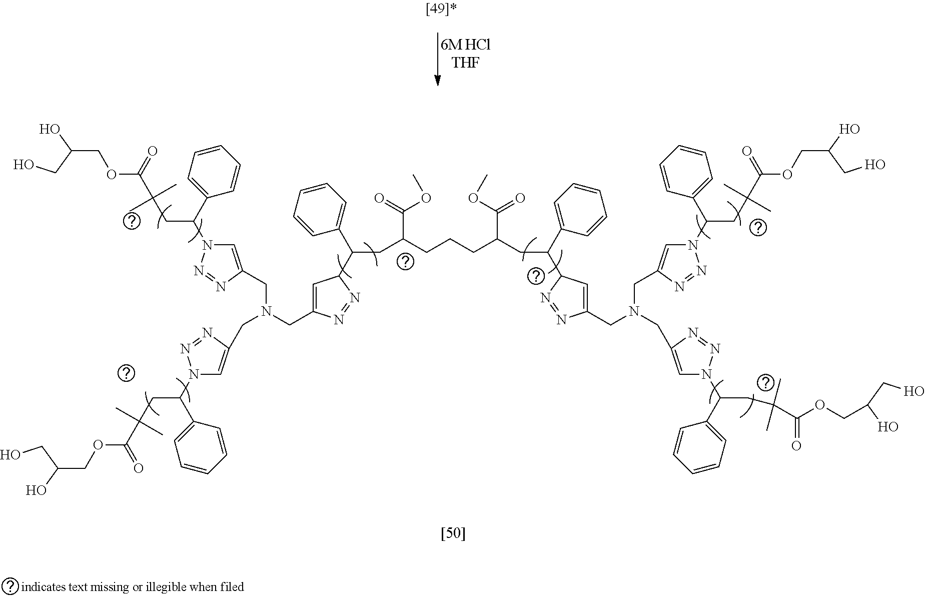

[49]* (0.55 g, 2.4×10⁻⁵ mol) was dissolved in 50 mL THF in a 100 mL conical flask equipped with magnetic stirrer. 6M HCl (1-2 mL) were added dropwise to the solution over a period of 5 min maintaining solubility of the polymer. The mixture was allowed to stir for 6 h at room temperature. The polymer [50] was precipitated in MeOH, filtered and dried for 24 h under vacuum.

### Synthesis of Sym-G₀-G₁-PSTY—Br [51]

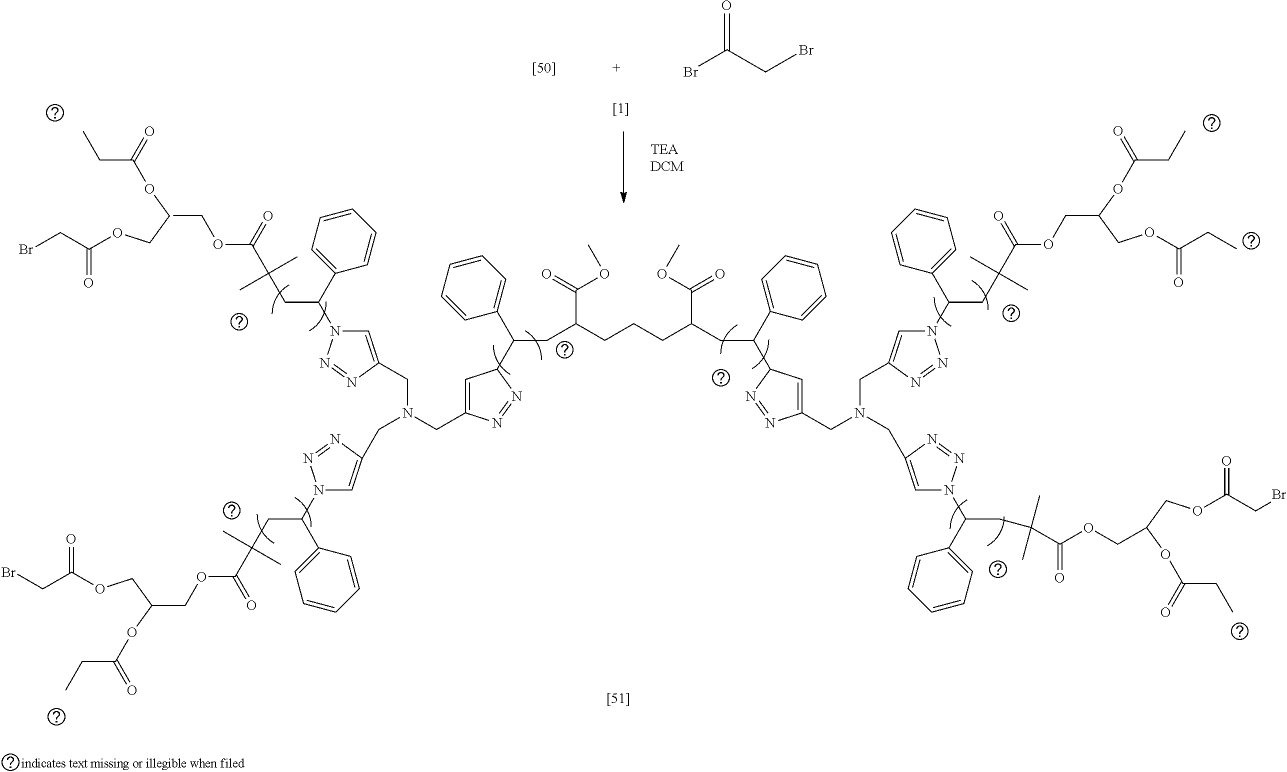

150] (0.5 g, 2.2×10⁻⁵ mol), triethylamine (0.026 mL, 1.9×10⁴ mol) in 10 mL of dry DCM were added to a 50 mL round bottom flask equipped with stirrer bar and pressure equalizing dropping funnel, under N₂. Bromoacetyl bromide ([1], 0.8 g, 8.8×10⁻⁴ mol) in 5 mL dry DCM was added dropwise to the stirred mixture over 10 min at room temperature. After complete addition, the mixture was stirred for 16 h. The polymer was precipitated in MeOH, then filtered and washed 3 times with MeOH (20 mL). The recovered polymer [51] was dried for 24 h under vacuum.

### Synthesis of Sym-G₀-G₁-PSTY—N₃ [52]

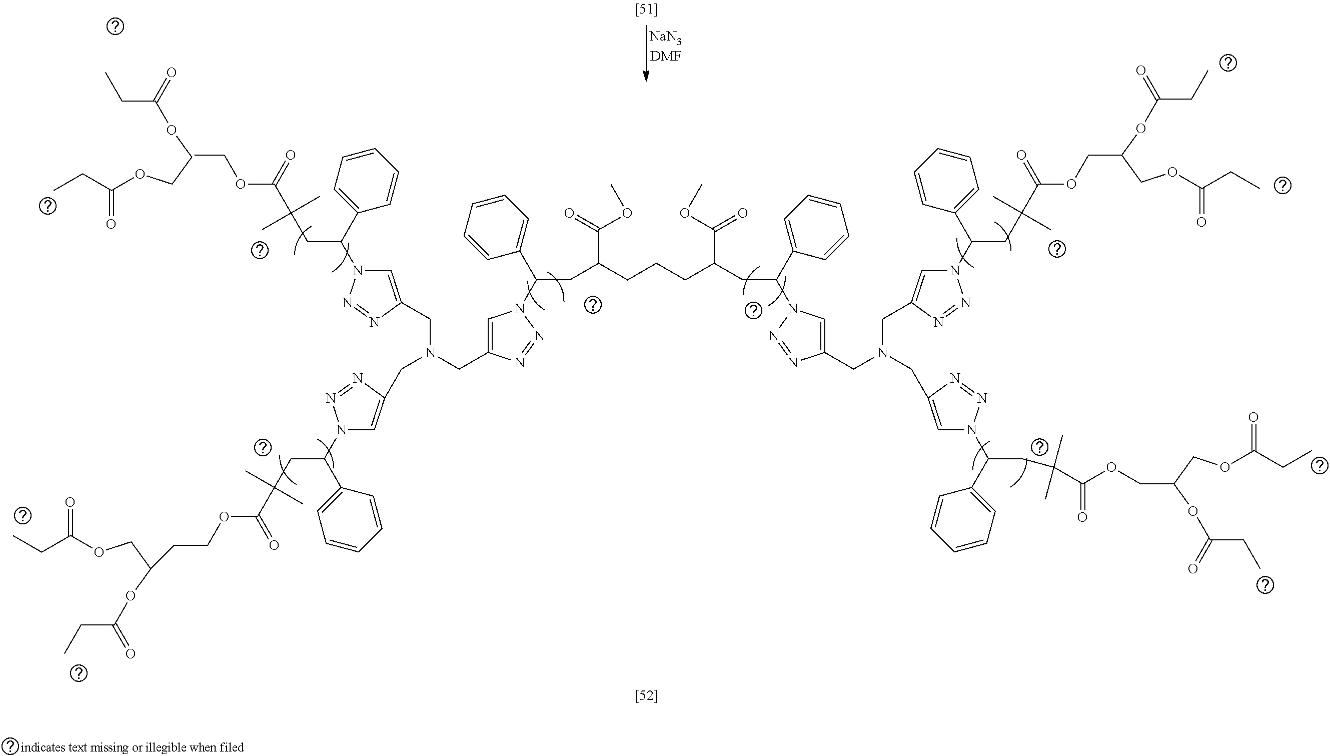

[51] (0.48 g, 2.1×10⁻⁵ mol) was dissolved in 5 mL of DMF. NaN₃ (0.108 g, 1.67×10⁻³ mol) was added and stirred for 24 h in a temperature controlled oil bath at 50° C. The polymer [52] was precipitated in MeOH, filtered and dried under vacuum for 48 h at 25° C.

### Synthesis of Sym-G₀-G₁-G₂-PSTY-Sol [53]

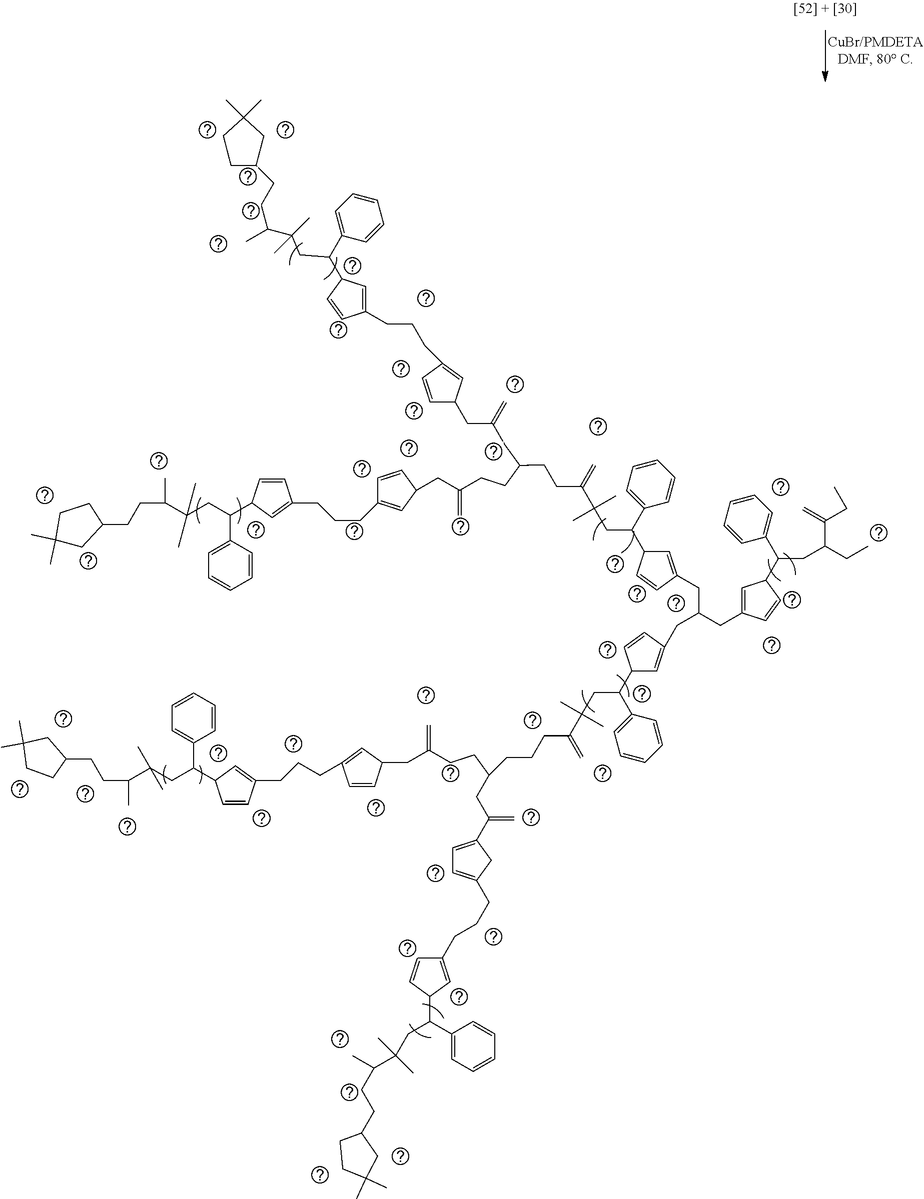

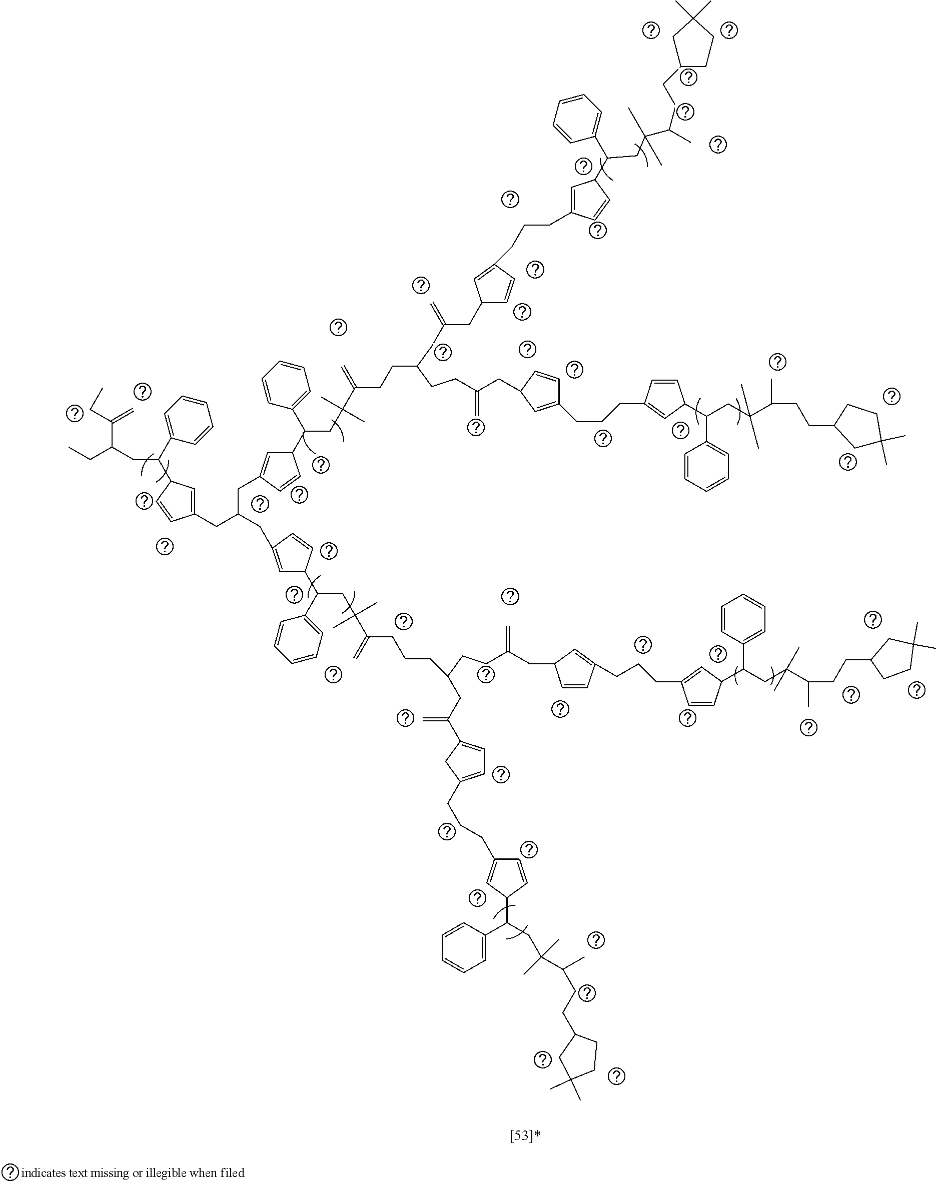

[52] (4.8 mg, 2.1×10⁴ mol), Sol-PSTY-(—≡) ([30], 8.6 mg, 1.8×10⁻⁶ mol), PMDETA (3.5 μL, 1.7×10⁻⁵ mol) in 0.5 mL of DMF was added to a 10 mL Schlenk flask, equipped with magnetic stirrer, and purged with N₂ for 10 min. CuBr (2.4 mg, 1.7×10⁻⁵ mol) was added under a positive flow of N₂, the flask was sealed and purged with N₂ for a further 5 min. The flask was placed in a temperature controlled oil bath at 80° C. for a period of 2 h. The reaction was diluted with 5 mL of THF then passed through activated basic alumina to remove the copper salts. The THF was removed by evaporation, and the polymer in residual DMF was added to a 10 mL Schlenk flask equipped with magnetic stirrer.

Further purification of starting material with azide groups from [53] was as follows: PMDETA (1.7 uL, 8.1×10⁻⁶ mol) and [41] (0.02 g) were added to the flask and the mixture purged with N₂ for 10 min. CuBr (1.2 mg, 8.3×10⁻⁶ mol) was added under a positive flow of N₂, the flask was sealed and purged with N₂ for a further 5 min. The flask was placed in a temperature controlled oil bath at 80° C. for 4 h to give [53]*.

This procedure was repeated for the synthesis of Sym-G₀-G₁-G₂-PSTY—(OH)₂ [54], Sym-G₀-G₁-PSTY-G₂-P^{t}BA-(OH)₂ [55] and Sym-G₀-G₁-G₂-PMA-(OH)₂ [56] using ≡—P(STY)—(OH)₂ [36], ≡—P^{t}BA-(OH)₂ [37] and ≡—PMA-(OH)₂ [38] respectively. However, in these cases, the beads [41] were not used to further purify the starting polymer from the product.

### Synthesis of Sym-G₀-G₁-G₂-PSTY—(OH)₂ [54]

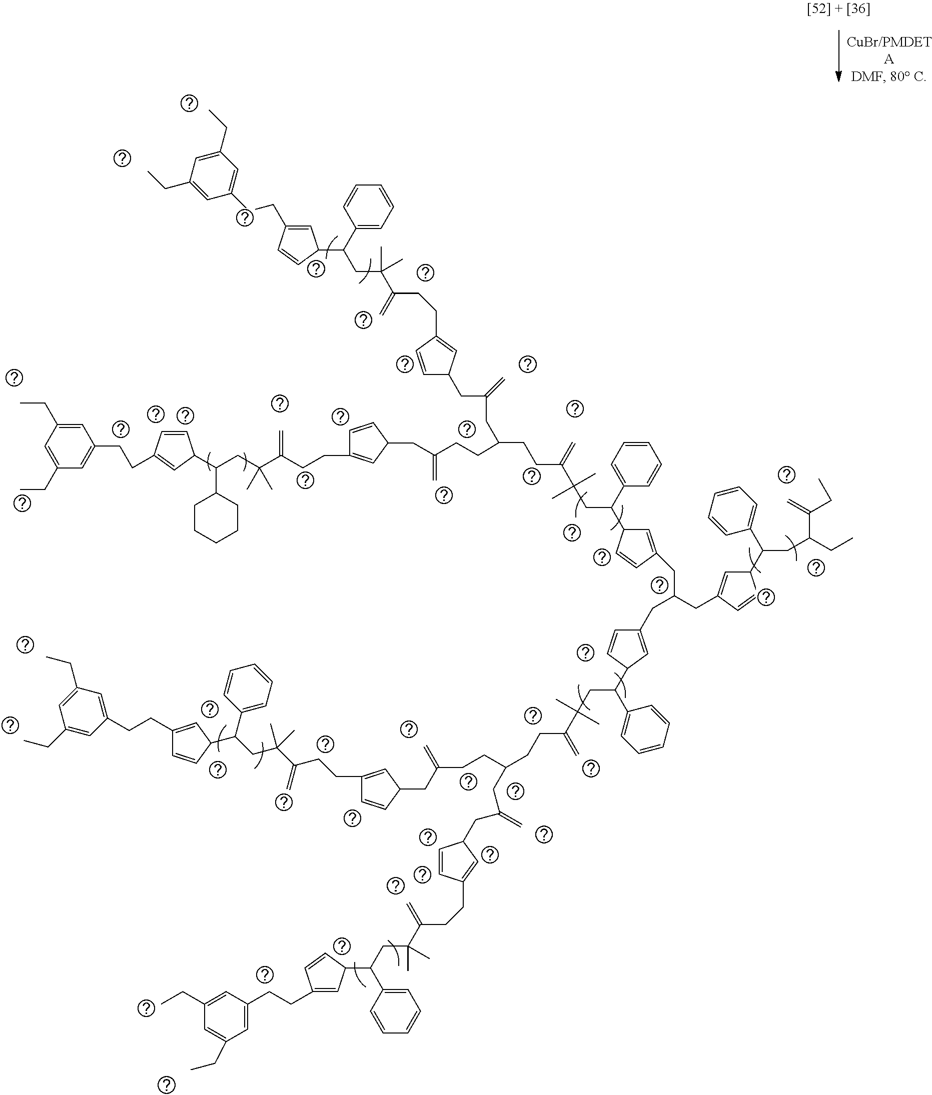

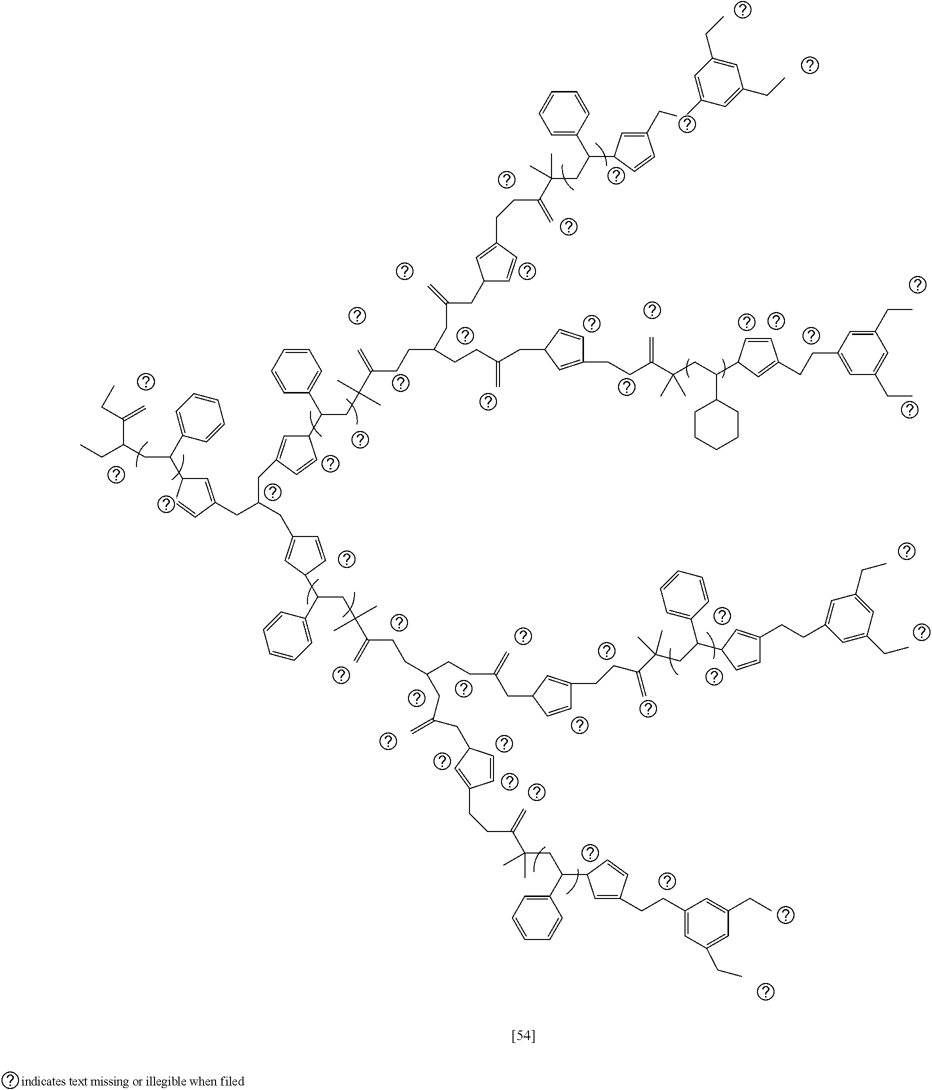

### Synthesis of Sym-G₀-G₁-G₂-P^{t}BA-(OH)₂ [55]

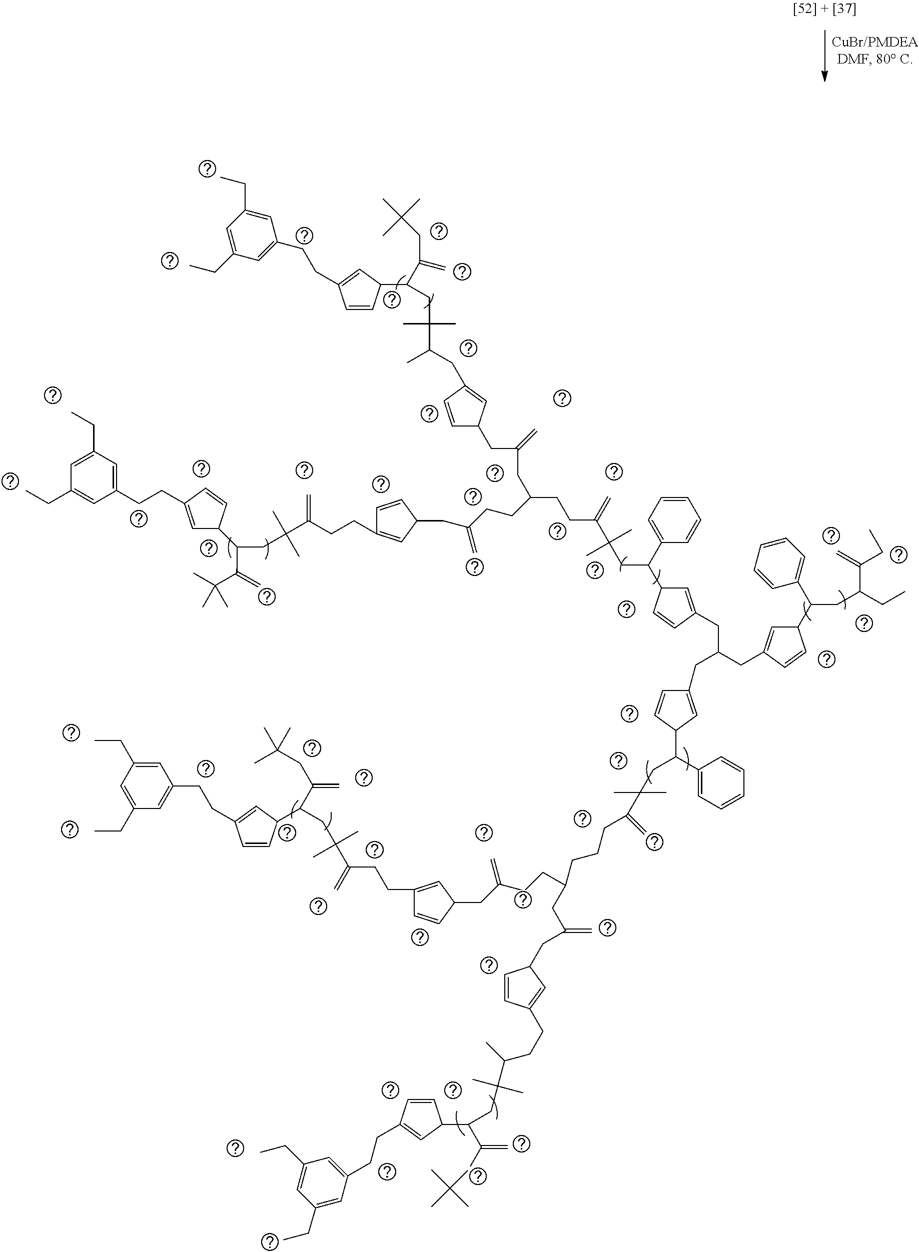

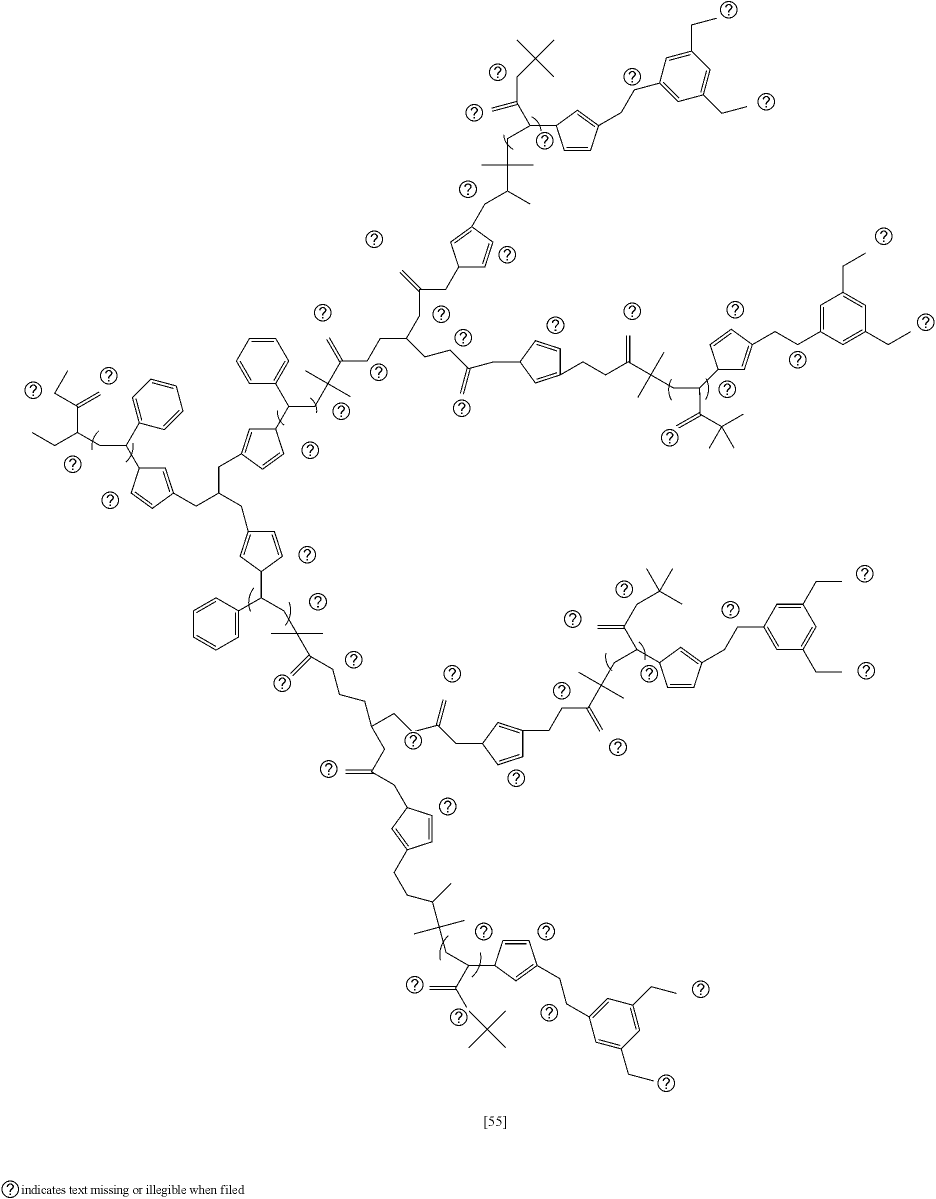

### Synthesis of Sym-G₀-G₁-PSTY-G₂-PMA-(OH)₂ [56]

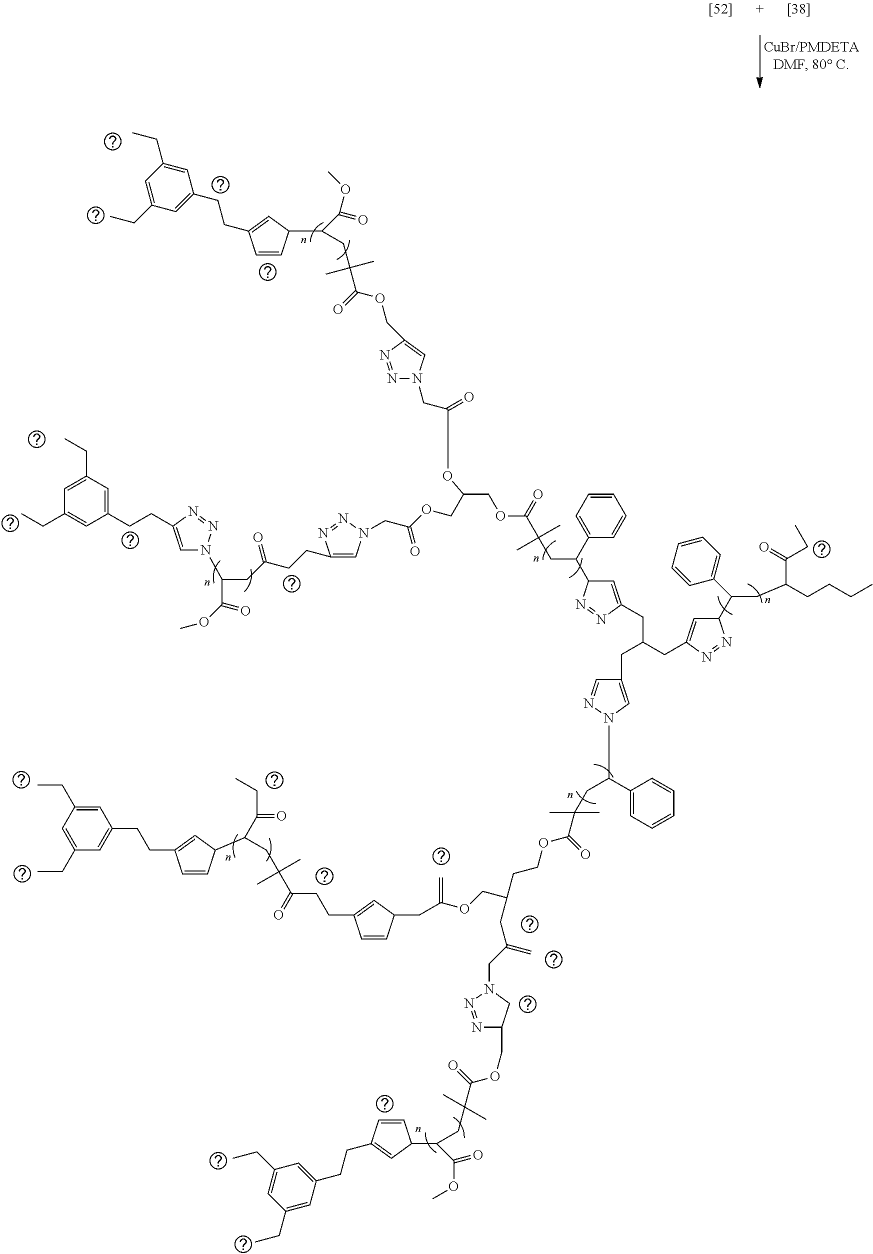

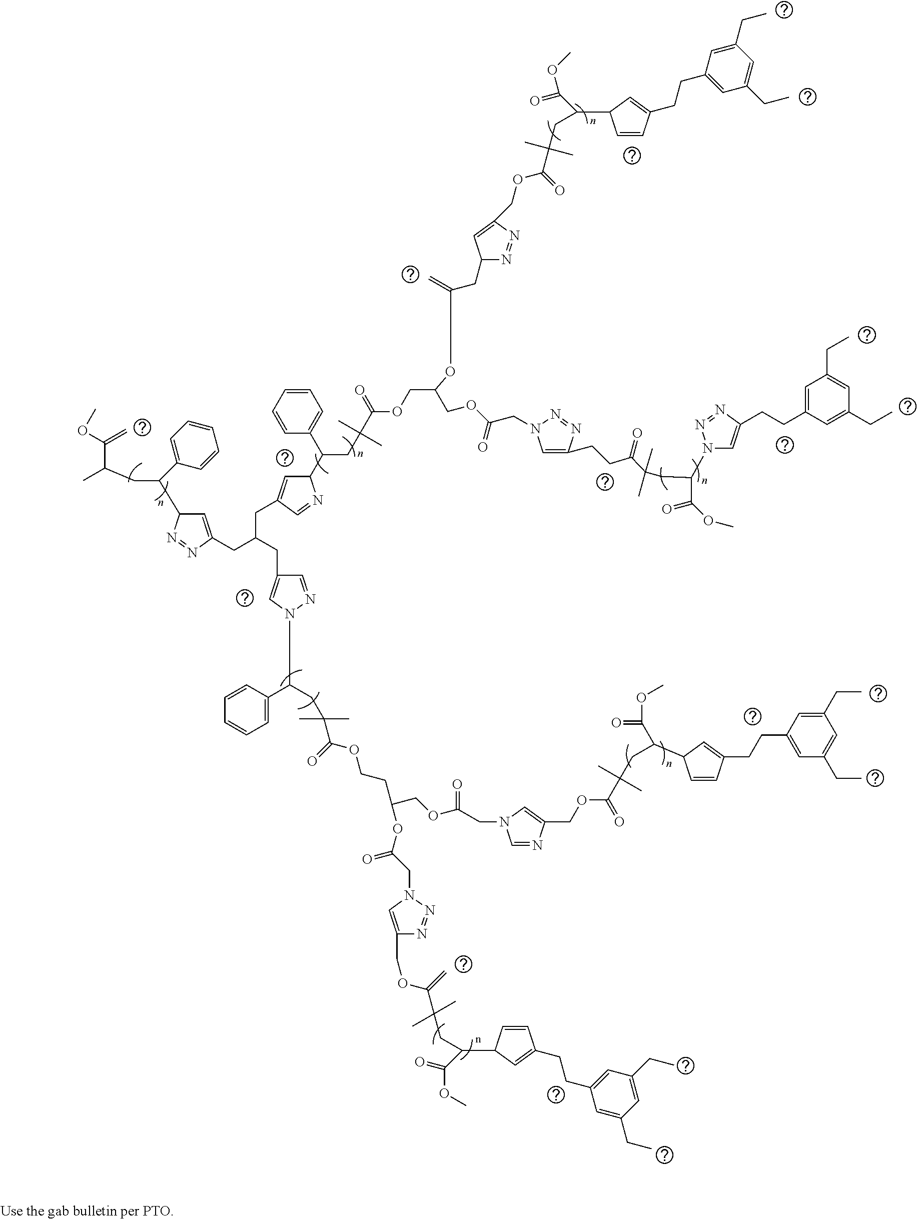

### Degradation of Sym-G₀-G₁-G₂-PSTY-Sol [53]

Where required, the symmetrical dendrimers of Example 7, in particular [53], can be degraded to obtain the constituent arms as follows:

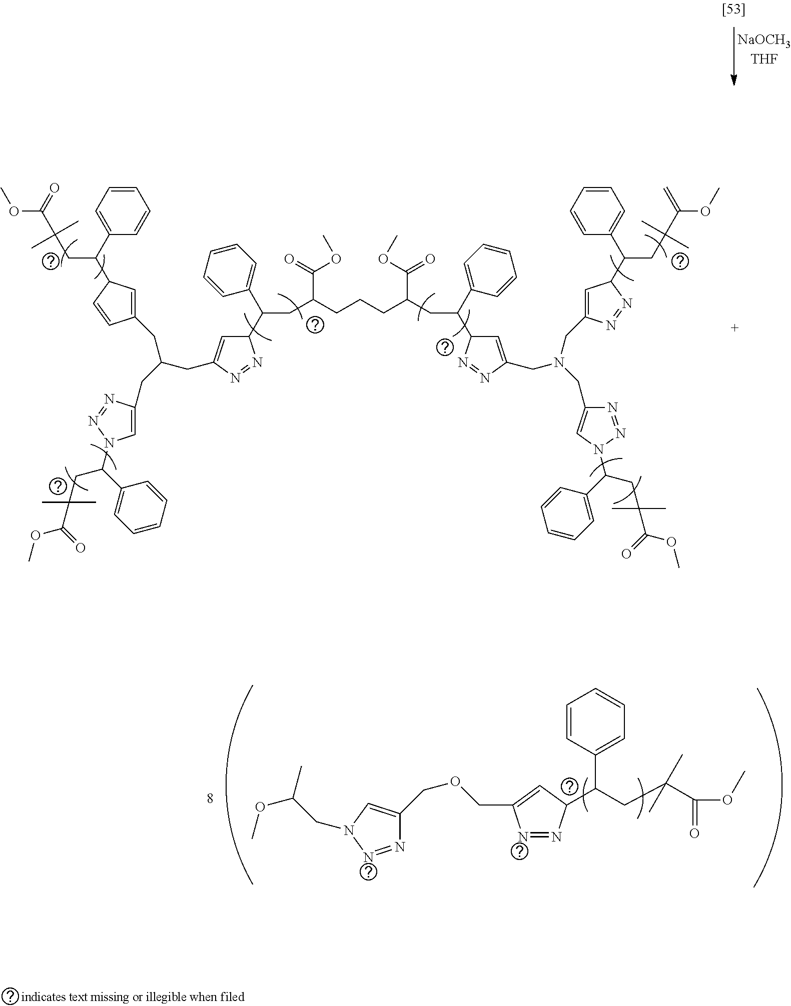

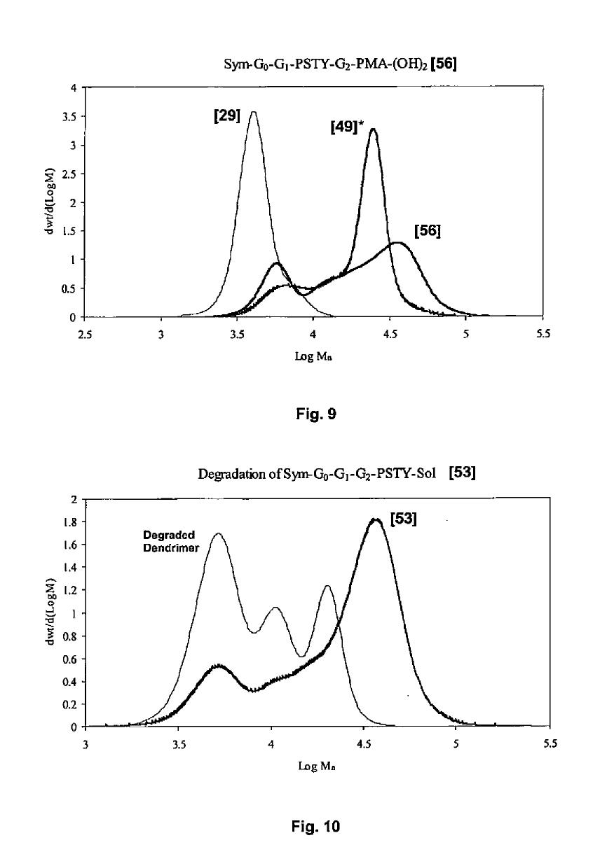

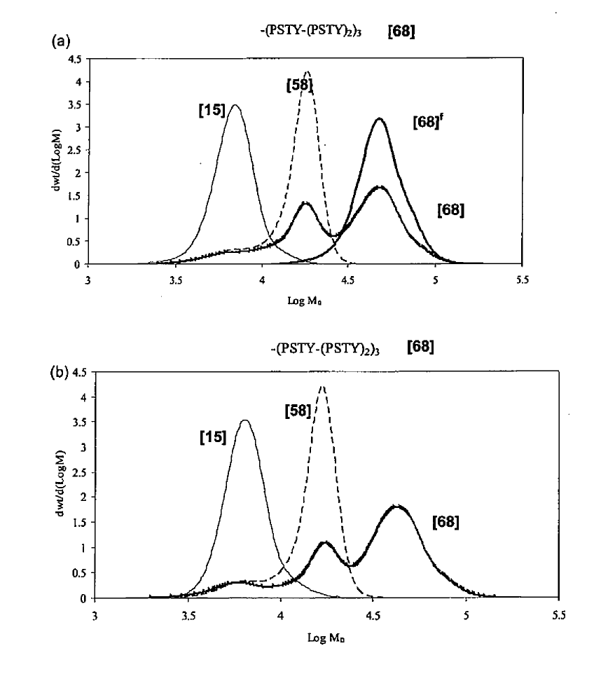

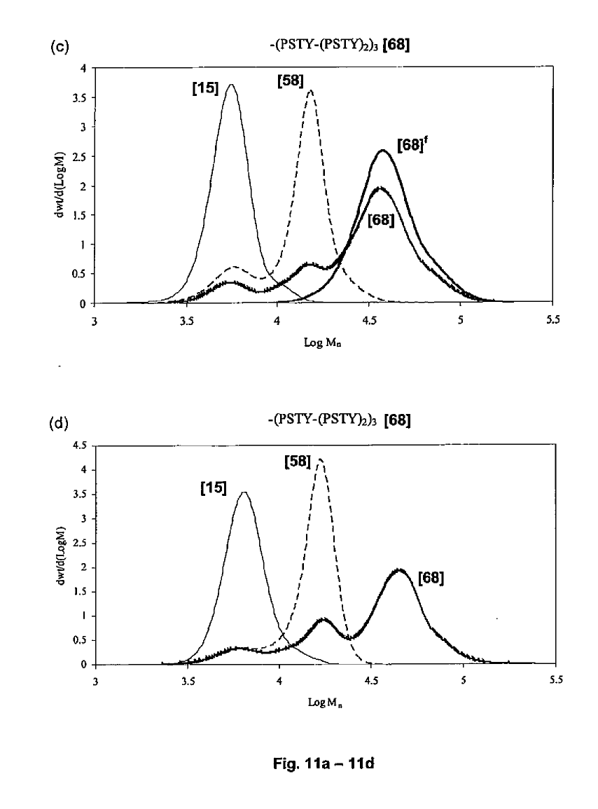

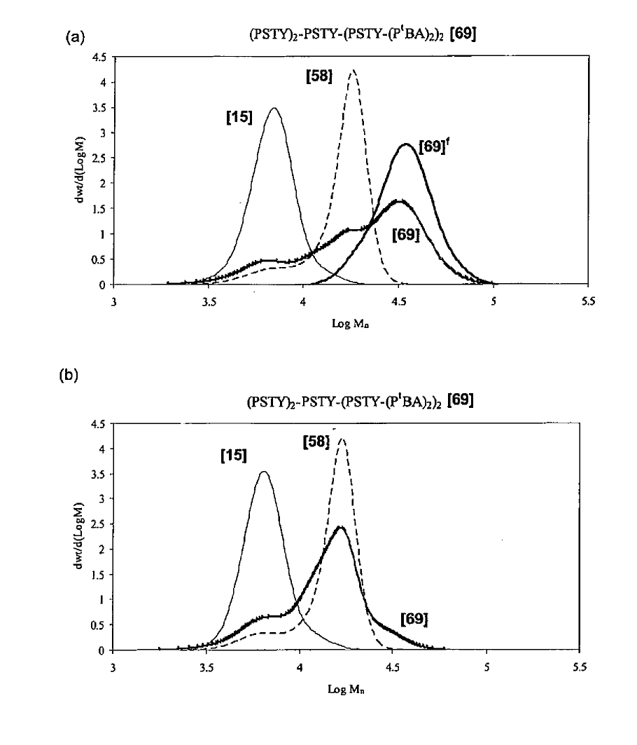

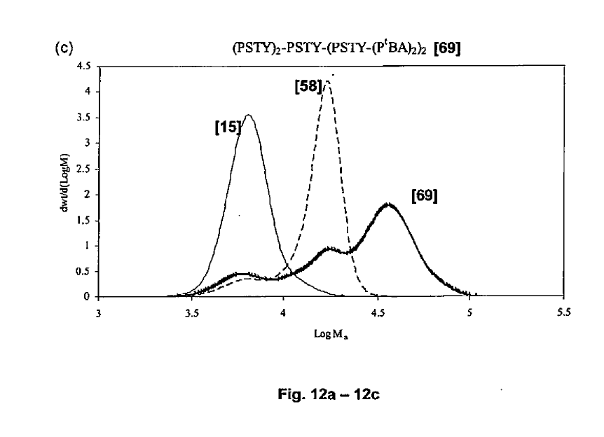

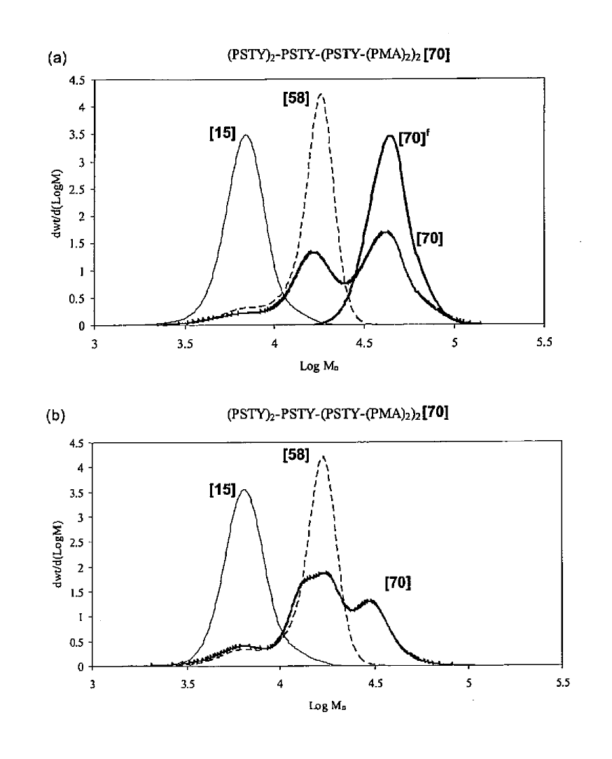

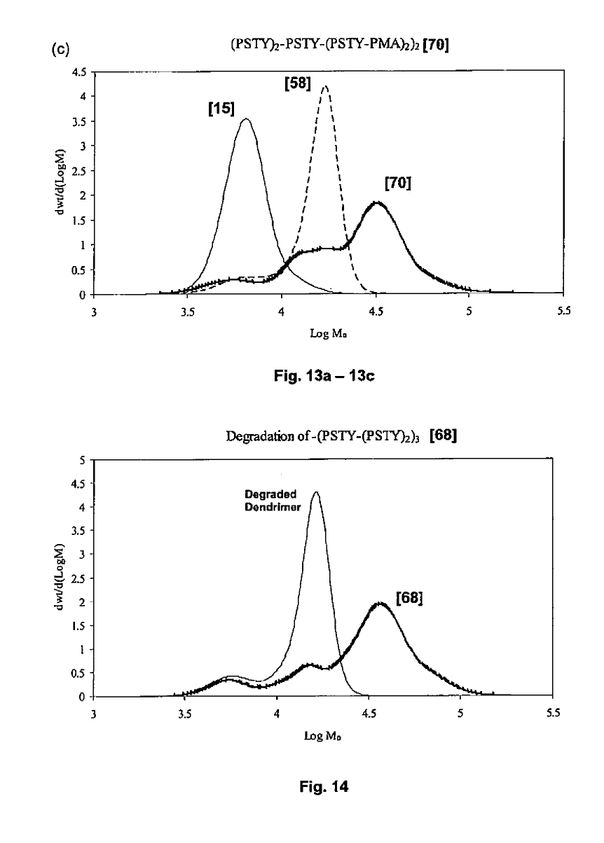

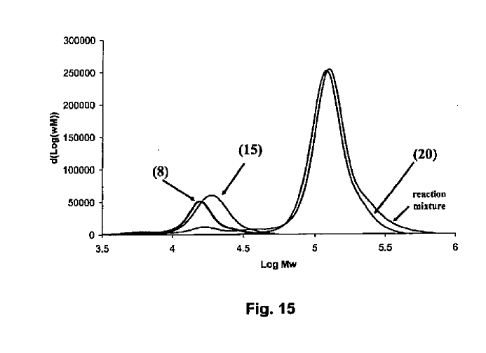

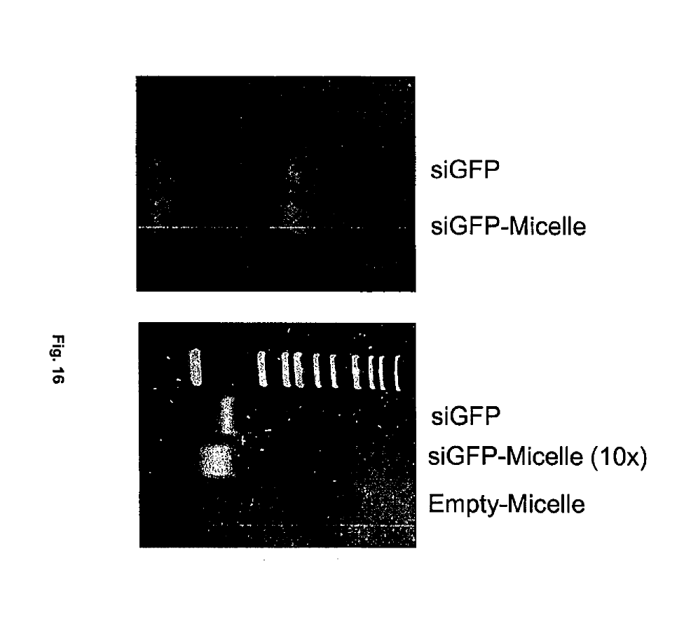

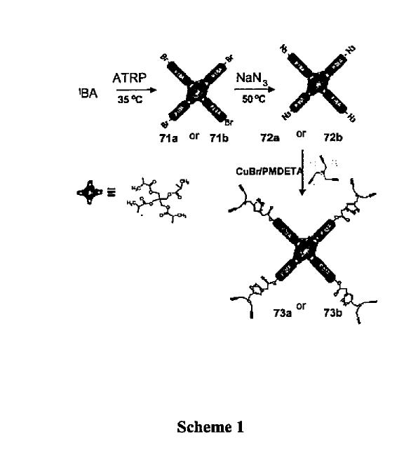

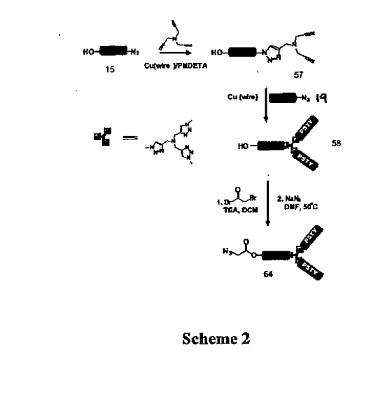

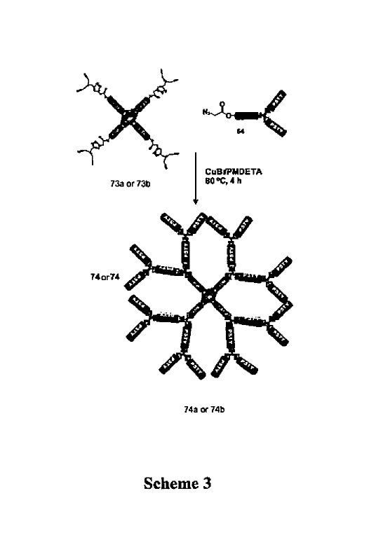

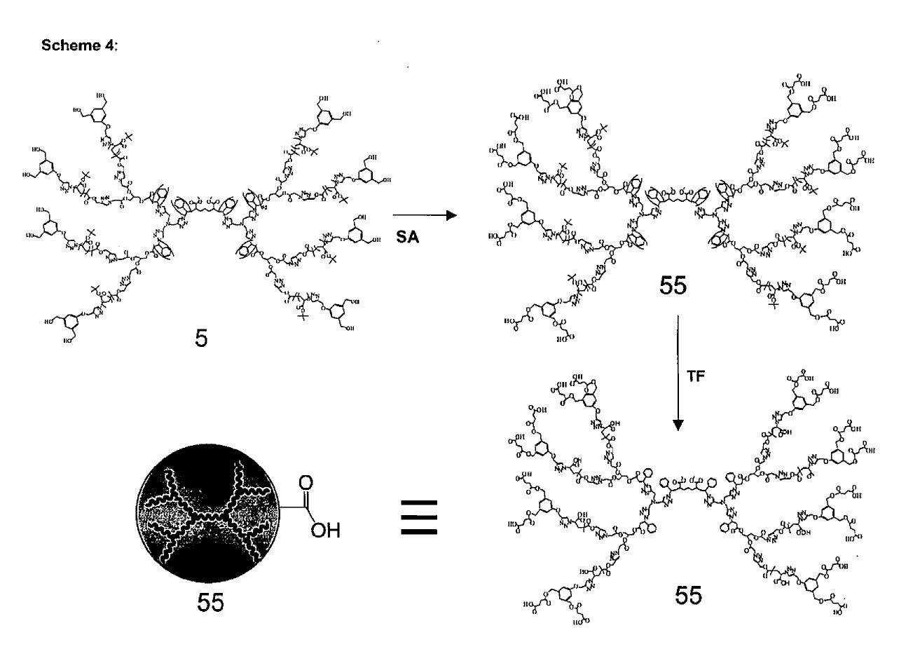

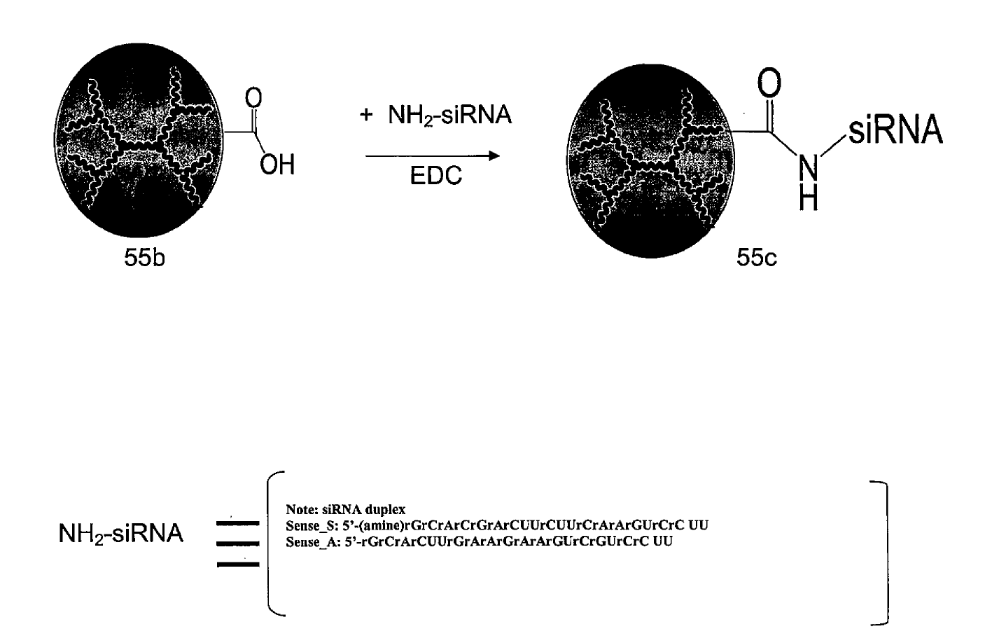

To a 250 μL aliquot of the reaction mixture from the synthesis of Sym-G₀-G₁-G₂-PSTY-Sol [53] was added THF (1 mL) and NaOCH₃ (10 mg, 1.85×10⁻⁴ mol). The mixture was stirred at room temperature for 16 h, then diluted and analysed by SEC as shown in FIG. 10.

The number average molecular weight (Mₙ), polydispersity index (PDI) and the yield of the symmetrical dendrimers of Example 7 are presented in tabular form below. As is clear from the table overleaf, the dendrimers have a narrow polydispersity.

Size Exclusion Chromatography Analyses

| Starting Functional Polymers/Dendrimers | | Dendrimers | Mₙ | PDI | Yield % |
|---|---|---|---|---|---|
| (≡—)₂—PSTY—(—≡)₂ [29] | Sol-PSTY—N₃ [23] | Sym-G₀-G₁-PSTY-Sol [49] | 20535 | 1.08 | 72 |
| (Mₙ = 3560, PDI = 1.11) | (Mₙ = 4650, PDI = 1.09) | Sym-G₀-G₁-PSTY-Sol [49]* | 18865 | 1.09 | 84 |
| Sym-G₀-G₁-PSTY—N₃ [52] | Sol-PSTY—(—≡) [30] | Sym-G₀-G₁-G₂-PSTY-Sol [53] | 33431 | 1.16 | 63 |
| (Mₙ = 18865, PDI = 1.09) | (Mₙ = 4650, PDI = 1.09) | Sym-G₀-G₁-G₂-PSTY-Sol [53]* | 32825 | 1.14 | 70 |
| Sym-G₀-G₁-PSTY—N₃ [52] | ≡—PSTY—(OH)₂ [36] | Sym-G₀-G₁-G₂-PSTY—(OH)₂ [54] | 45684 | 1.19 | 73 |
| (Mₙ = 18865, PDI = 1.09) | (Mₙ = 4651, PDI = 1.09) | | | | |
| Sym-G₀-G₁-PSTY—N₃ [52] | ≡—P^{t}BA—(OH)₂ [37] | Sym-G₀-G₁-PSTY-G₂-P^{t}BA—(OH)₂ [55] | 31426 | 1.14 | 65 |
| (Mₙ = 18865, PDI = 1.09) | (Mₙ = 4200, PDI = 1.11) | | | | |

### Example 8

### Mikto-Arm Star Dendrimers

### Synthesis of Functional Arm HO—PSTY-(—≡)₂ [57]

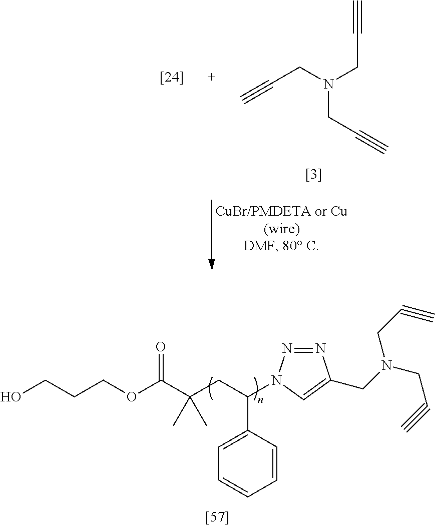

Method A. 10×CuBr/PMDETA

HO—PSTY—N₃ ([24], 1.120 g, 1.78×10⁻⁴ mol), PMDETA (0.284 g, 1.64×10⁻³ mol), and tripropargylamine ([3], 0.431 g, 3.29×10⁻³ mol) in 10 mL of DMF was added to a 10 mL Schlenk flask, and purged with N₂ for 10 min. CuBr (0.233 g, 1.63×10⁻³ mol) was added under a positive flow of N₂, the flask was sealed and purged with N₂ for a further 5 min. The flask was placed in a temperature controlled oil bath at 80° C. for a period of 2 h. The solution was then diluted with THF, and passed through a basic alumina column. The solution was concentrated under N₂ flow and the polymer recovered by precipitation into cold MeOH and then filtered. The polymer [57] was redissolved in DMF (5 mL) and re-precipitated into cold MeOH, filtered and dried under vacuum.

Method B: 0.5×CuBr/PMDETA

HO—PSTY—N₃ ([24], 0.4385 g, 7.01×10⁻⁵ mol), PMDETA (0.0061 g, 3.49×10⁻⁵ mol), and tripropargylamine ([3], 0.184 g, 1.40×10⁻³ mol) in 4.4 mL of DMF was added to a 10 mL Schlenk flask, and purged with N₂ for 10 min. CuBr (0.0051 g, 3.55×10⁻⁵ mol) was added under a positive flow of N₂, the flask was sealed and purged with N₂ for a further 5 min. The flask was placed in a temperature controlled oil bath at 80° C. for a period of 2 h. The polymer was recovered by precipitation into MeOH and then filtered. The polymer [57] was redissolved in DMF (4 mL) and re-precipitated into MeOH, filtered and dried under vacuum.

Method C: Cu (Wire)

HO—PSTY—N₃ ([24], 0.6427 g, 1.17×10⁻⁴ mol), PMDETA (0.0101 g, 5.84×10⁻⁵ mol), tripropargylamine ([3], 0.306 g, 2.33×10⁻⁴ mol) and Cu (wire, 0.30 g) in 6.4 mL of DMF was added to a 10 mL Schlenk flask. The flask was placed in a temperature controlled oil bath at 80° C. for a period of 4 h. The polymer was recovered by precipitation into MeOH and then filtered. The polymer [57] was redissolved in DMF (6 mL) and re-precipitated into MeOH, filtered and dried under vacuum.

### Synthesis of Functional Arm Star HO—PSTY—(PSTY)₂ [58]

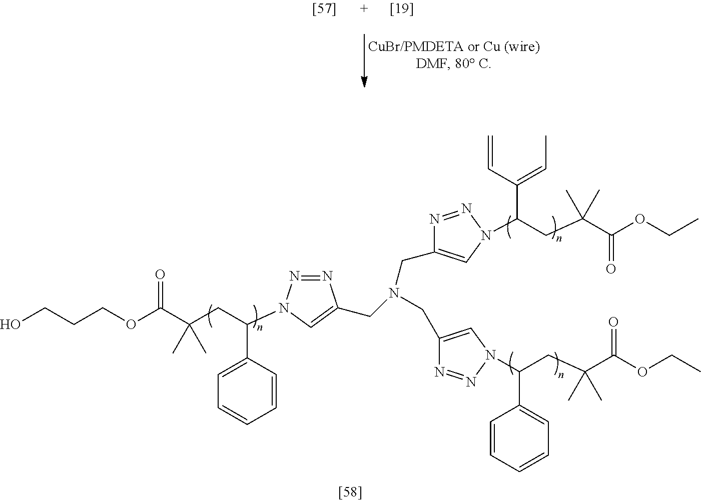

Method (A) 10×CuBr/PMDETA

[57] (prepared by Method A, 0.300 g, 4.79×10⁻⁵ mol), PSTY—N₃ ([19], 0.528 g, 1.03×10⁻⁴ mol), and PMDETA (0.163 g, 9.40×10⁻⁴ mol) in 8 mL of DMF were added to a 10 mL Schlenk flask, and purged with N₂ for 10 min. CuBr (133 mg, 9.27×10⁻⁴ mol) was added under a positive flow of N₂, the flask was then sealed and purged with N₂ for a further 5 min. The flask was placed in a temperature controlled oil bath at 80° C. for 2 h. The solution was diluted with THF and passed through a basic alumina column. The solution was concentrated under N₂ flow, and the polymer [58] precipitated into cold methanol, filtered and dried under vacuum.

The above procedure was repeated for the synthesis of the functional mikto-arm stars HO—PSTY—(P^{t})₂ [59] and HO—PSTY—(PMA)₂ [60] using P^{t}BA-N₃ [21] and PMA-N₃ [20] respectively.

Method (B) 0.5×CuBr/PMDETA

[57] (prepared by Method B, 0.2198 g, 3.55×10⁻⁵ mol), PSTY—N₃ ([19], 0.3961 g, 7.73×10⁻⁵ mol), and PMDETA (0.0066 g, 3.83×10⁻⁵ mol) in 6 mL of DMF were added to a 10 mL Schlenk flask, and purged with N₂ for 10 min. CuBr (0.0055 g, 3.83×10⁻⁵ mol)was added under a positive flow of N₂, the flask was then sealed and purged with N₂ for a further 5 min. The flask was placed in a temperature controlled oil bath at 80° C. for 3 h. The polymer [58] was precipitated into cold methanol, filtered and dried under vacuum.

The above procedure was repeated for the synthesis of the functional mikto-arm stars HO—PSTY—(P^{t}BA)₂ [59] and HO—PSTY—(PMA)₂ [60] using P^{t}BA-N₃ [21] and PMA-N₃ [20] respectively. These polymers were purified by precipitation into water, then filtered and dried under vacuum.

Method (C) Cu (Wire)

[57] (prepared by Method C, 0.198 g, 3.60×10⁻⁵ mol), PSTY—N₃ ([19], 0,4234 g, 7.56×10⁻⁵ mol), and Cu (wire, 1.0 g) in 6 mL of DMF were added to a 10 mL Schlenk flask. The flask was placed in a temperature controlled oil bath at 80° C. for 4 h. The polymer [58] was precipitated into cold methanol, filtered and dried under vacuum.

### Synthesis of Functional Arm Star HO—PSTY—(P^{t}BA)₂ [59]

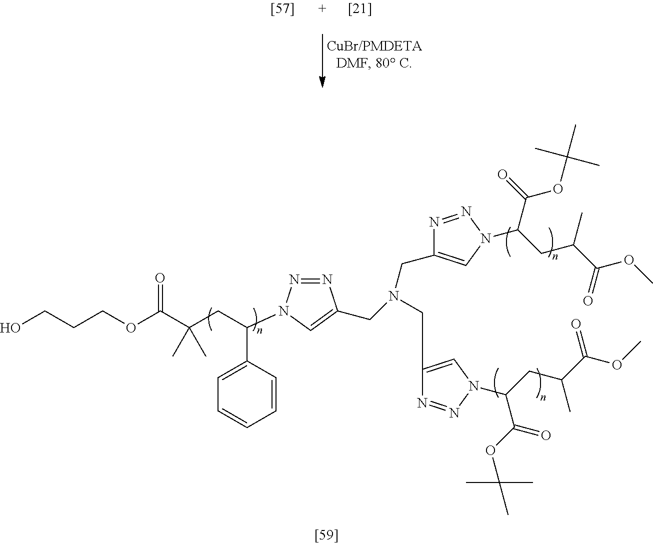

### Synthesis of Functional Arm Star HO—PSTY—(PMA)₂ [60]

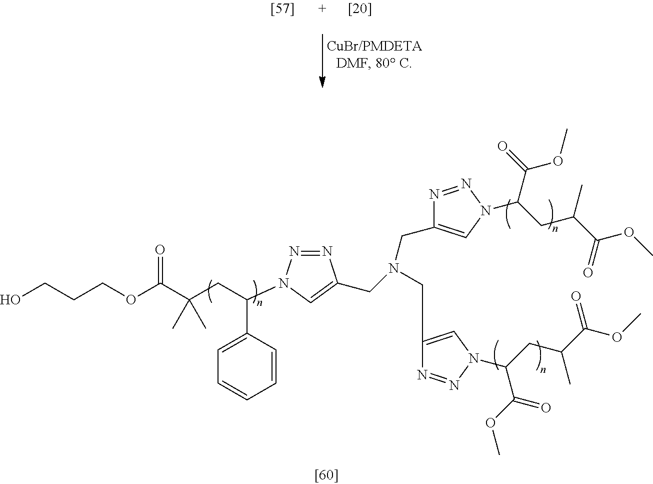

### Synthesis of Functional Arm Star Br—PSTY—(PSTY)₂ [61]

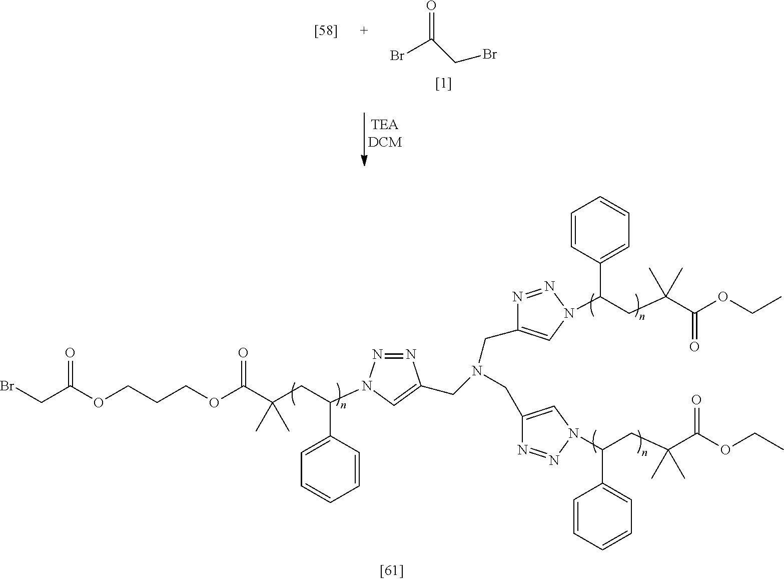

[58] (500 mg, 2.94×10⁻⁵ mol), triethylamine (4.5 μL, 3.2×10⁻⁵ mol), in 1.5 mL of dry DCM was added to a 10 mL Schlenk flask equipped with stirrer bar, under N₂. Bromoacetyl bromide (12.7 μL, 1.47×10⁻⁴ mol) in 0.5 mL of dry DCM was added dropwise to the stirred mixture over a period of 20 min at room temperature. After complete addition the mixture was allowed to stir for 16 h. The polymer was precipitated in MeOH, then filtered and washed 3 times with MeOH (20 mL). The recovered polymer [61] was dried for 24 h under vacuum.

The above procedure was repeated for the synthesis of the functional mikto-arm stars Br—PSTY—(P^{t}BA)₂ [62] and Br—PSTY—(PMA)₂ [63] using HO—PSTY—(P^{t}BA)₂ [59] and HO—PSTY—(PMA)₂ [60] respectively.

### Synthesis of Functional Arm Star Br—PSTY—(P^{t}BA)₂ [62]

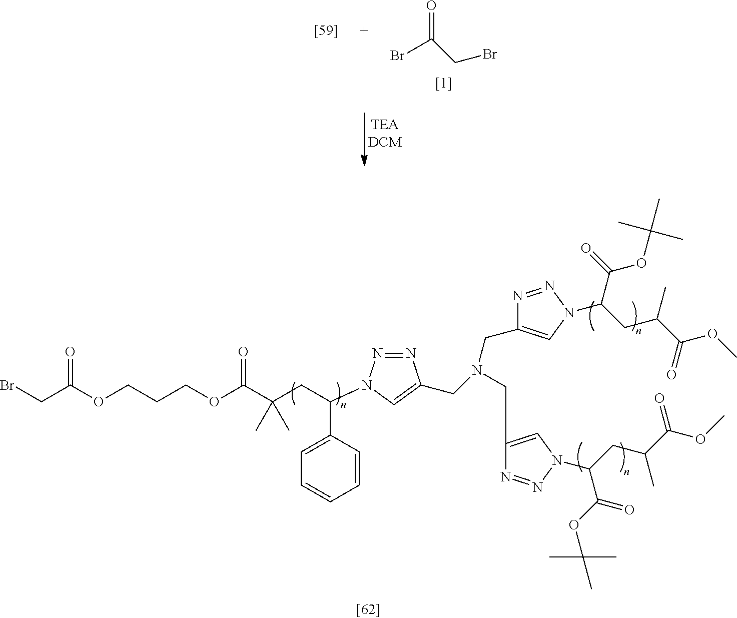

### Synthesis of Functional Arm Star Br—PSTY—(PMA)₂ [63]

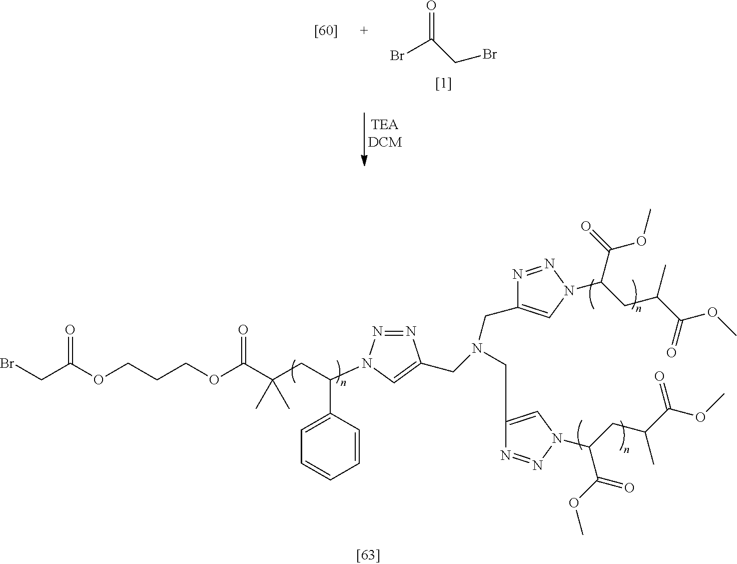

### Synthesis of Functional Arm Star N₃—PSTY—(PSTY)₂ [64]

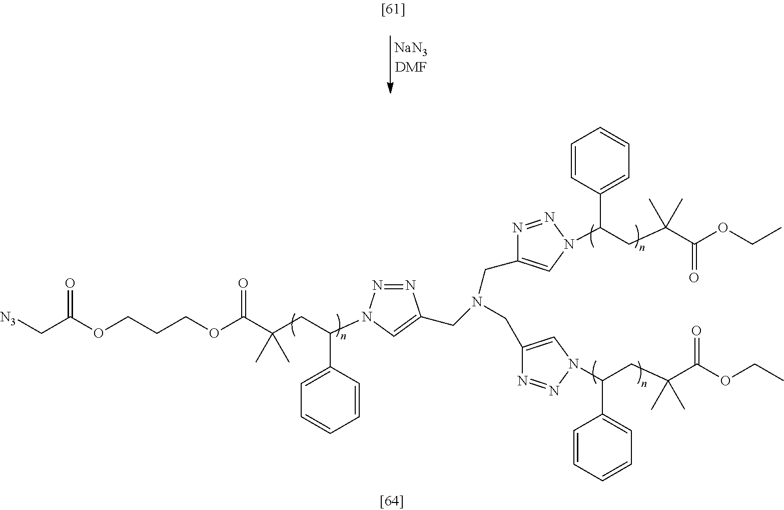

[61] (0.450 g, 2.67×10⁻⁵ mol) was dissolved in 2 mL DME. NaN₃ (0.0166 g, 2.67×10⁻⁴ mol) was added and stirred for 24 h in a temperature controlled oil bath set at 50° C. The polymer [64] was precipitated in water with vigorous stirring, filtered and dried under high vacuum.

The above procedure was used for the synthesis of the functional mikto-arm stars N₃—PSTY—(P^{t}BA)₂ [65] and N₃—PSTY—(PMA)₂ [66] using Br—PSTY—(P^{t}BA)₂ [62] and Br—PSTY—(PMA)₂ [63].

### Synthesis of Functional Arm Star N₃—PSTY—(P^{t}BA)₂ [65]

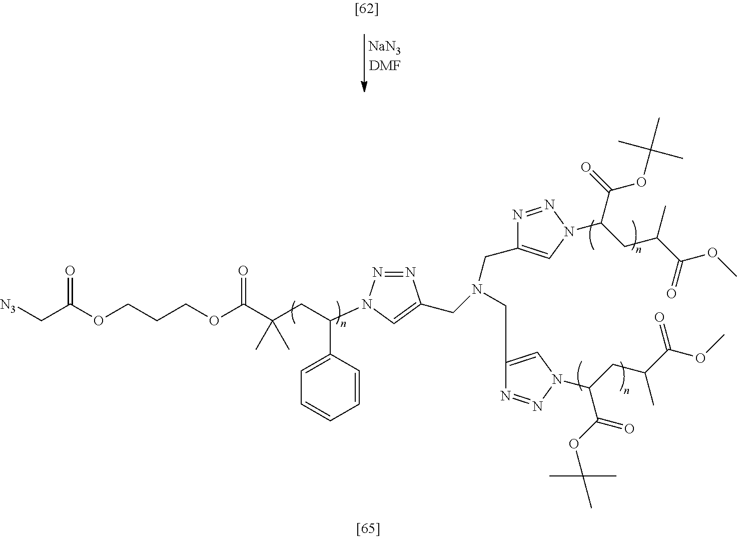

### Synthesis of Functional Arm Star N₃—PSTY—(PMA)₂ [66]

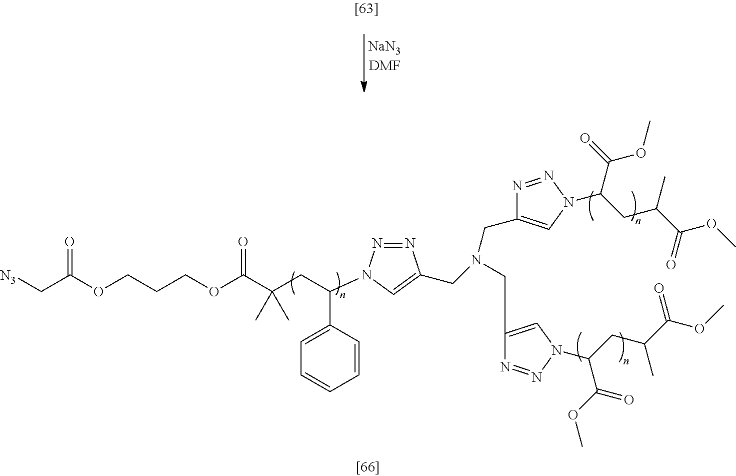

### Synthesis of Functional Centre Star (≡—)₂-PSTY—(PSTY)₂ [67]

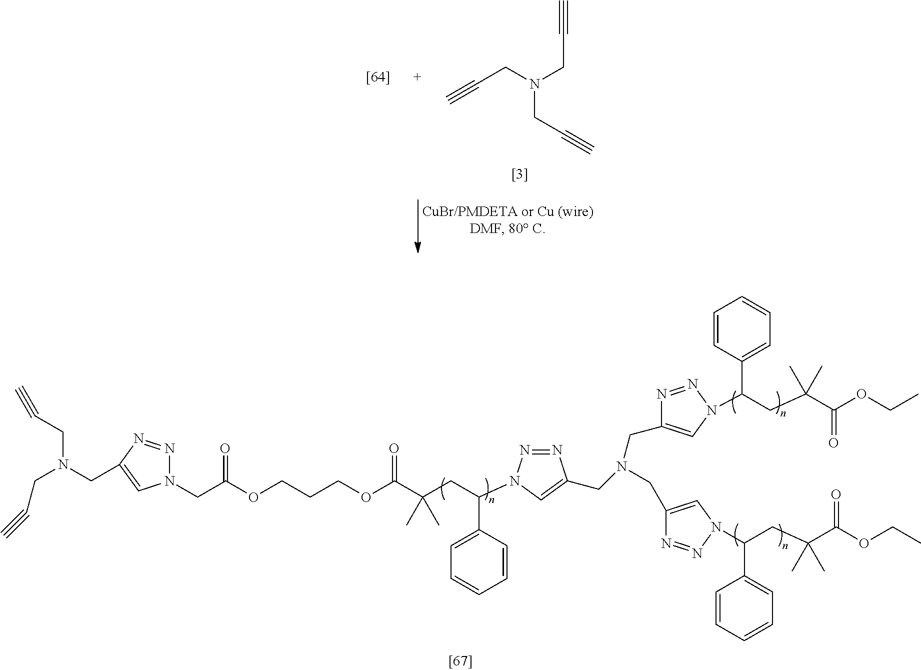

Method A: 10×CuBr/PMDETA

[64] (prepared by Method A, 0.200 g, 1.18×10⁻⁵ mol), PMDETA (25 μL, 1.20×10⁻⁴ mol), and tripropargylamine an 34 μL, 2.35×10⁻⁴ mol) in 2 mL of DMF was added to a 10 mL Schlenk flask, equipped with magnetic stirrer, and purged with N₂ for 20 min. CuBr (0.0168 g, 1.17×10⁻⁴ mol) was added under a positive flow of N₂, the flask was sealed and purged with N₂ for a further 5 min. The flask was placed in a temperature controlled oil bath set at 80° C. for 2 h. The solution was then diluted with THF and passed through a basic alumina column. The solution was concentrated under N₂ flow, and the polymer precipitated into cold methanol then filtered. The polymer [67] was redissolved in DMF (5 mL) and re-precipitated in cold MeOH, filtered and dried under vacuum.

Method B: 0.5×CuBr/PMDETA

[64] (prepared by Method B, 0.1498 g, 9.25×10⁻⁶ mol) and tripropargylamine ([3], 26 μL, 1.84×10⁻⁴ mol) in 1 mL of DMF was added to a 10 mL Schlenk flask, equipped with magnetic stirrer, and purged with N₂ for 20 min. A 100 μL aliquot of a deoxygenated solution containing, PMDETA (49 μL, 2.34×10⁻⁴ mol) and CuBr (0.033 g, 2.30×10⁻⁴ mol) in 5 mL DMF was added to the Schlenk flask, under positive N₂ flow, the flask was sealed and purged with N₂ for a further 5 min. The flask was placed in a temperature controlled oil bath at 80° C. for 3 h. The polymer was precipitated into cold methanol then filtered. The polymer [67] was redissolved in DMF (2 mL) and re-precipitated in cold MeOH, filtered and dried under vacuum.

Method C: Cu (Wire)

[64] (prepared by Method C, 0.2366 g, 1.34×10 mol), tripropargylamine ([3], 38 μL, 2.69×10⁻⁴ mol) and Cu (wire, 0.355 g) in 2.5 mL of DMF was added to a 10 mL Schlenk flask, equipped with magnetic stirrer. The flask was placed in a temperature controlled oil bath at 80° C. for 4 h. The polymer was precipitated into cold methanol then filtered. The polymer [67] was redissolved in DMF (2.5 mL) and re-precipitated in cold MeOH, filtered and dried under vacuum.

### Synthesis of 3-Arm Dendritic Star —(PSTY—(PSTY)₂)₃ [68]

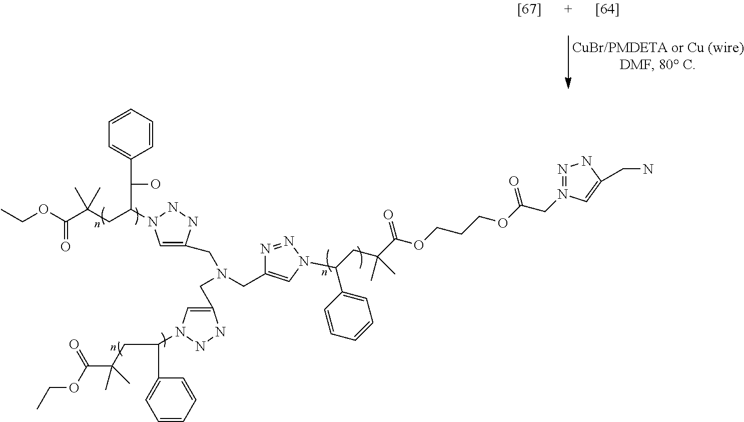

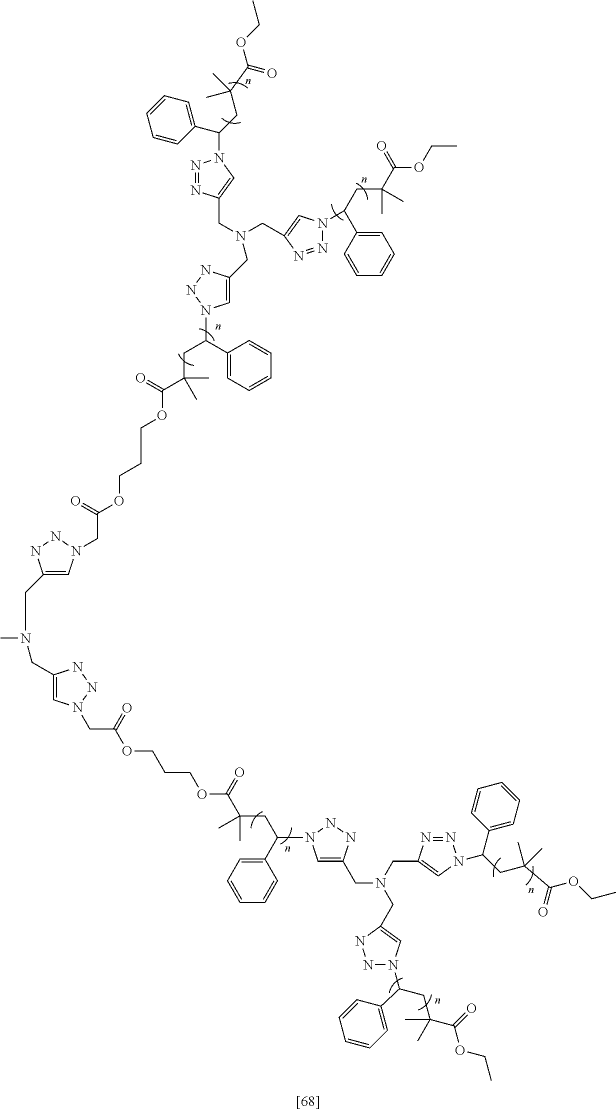

Method A: 10×CuBr/PMDETA

[67] (prepared by Method A, 5.3 mg, 3.12×10⁻⁷ mol), [64] (11.2 mg, 6.59×10⁻⁷ mol), and PMDETA (1.4 μL, 6.47×10⁻⁶ mol) in 0.5 mL of DMF was added to a 10 mL Schlenk flask, and purged with N₂ for 10 min. CuBr (1.3 mg, 9.1×10⁻⁶ mol) was added under a positive flow of N₂, the flask was sealed and purged with N₂ for a further 5 min. The flask was placed in a temperature controlled oil bath at 80° C. for 2 h, then a sample removed for SEC analysis. The dendritic star was then purified from the bulk reaction by fractionation using SEC. The fractionated polymer [68]^{f} was then analysed by SEC.

The above procedure was repeated for the synthesis of the functional mikto-arm dendritic stars (PSTY)₂—PSTY—(PSTY—(P^{t}BA)₂)₂ [69] and [69]^{f} and (PSTY)2—PSTY—(PSTY—(PMA)₂)₂ [70] and [70]^{f} using N₃—PSTY—(P^{t}BA)₂ [65] and N₃—PSTY—(PMA)₂ [66] respectively.

Method B: 0.5×CuBr/PMDETA

[67] (prepared by Method B, 4.9 mg, 3.03×10⁻⁷ mol) and [64] (prepared by Method B, 10.8 mg, 6.67×10⁻⁷ mol) in 0.5 mL of DMF was added to a 10 mL Schlenk flask, and purged with N₂ for 10 min. A 100 μL aliquot of a deoxygenated solution containing PMDETA (7 μL, 3.35×10⁻⁵ mol) and CuBr (4.8 mg, 3.35×10⁻⁵ mol) in 10 mL DMF was added to the Schlenk flask, under positive N₂ flow, the flask was sealed and purged with N₂ for a further 5 min. The flask was placed in a temperature controlled oil bath at 80° C. for 4 h, and then a sample removed for SEC analysis.

The above procedure was repeated for the synthesis of the functional mikto-arm dendritic stars (PSTY)₂—PSTY—(PSTY—(P^{t}BA)₂)₂ [69] and (PSTY)₂—PSTY—(PSTY—(PMA)₂)₂ [70] using N₃—PSTY—(P^{t}BA)₂ [65] and N₃—PSTY—(PMA)₂ [66] respectively. The reactions were maintained at 80° C. for 19 h.

Method C: Cu (Wire)

[67] (prepared by Method C, 5.0 mg, 2.82×10⁻⁷ mol), [64] (10.5 mg, 5.93×10⁻⁷ mol), and Cu (wire, 50.0 mg) in 0.5 mL of DMF was added to a 10 mL Schlenk flask, equipped with magnetic stirrer. The flask was placed in a temperature controlled oil bath at 80° C. for 4 h, and then a sample removed for SEC analysis. The dendritic star was then purified from the bulk reaction by fractionation using SEC. The fractionated polymer [68]^{f} was then analysed by SEC.

The above procedure was repeated for the synthesis of the functional homo and mikto-arm dendritic stars —(PSTY—(PSTY)₂)₃ [68], (PSTY)₂—PSTY—(PSTY—(P^{t}BA)₂)₂ [69] and (PSTY)₂—PSTY—(PSTY—(PMA)₂)₂ [70] using the following polymers prepared by Method B: [67] and [64], [67] and [65] and [67] and [66] respectively.

### Synthesis of (PSTY)₂—PSTY—(PSTY—(P^{t}BA₂))₂ [69]

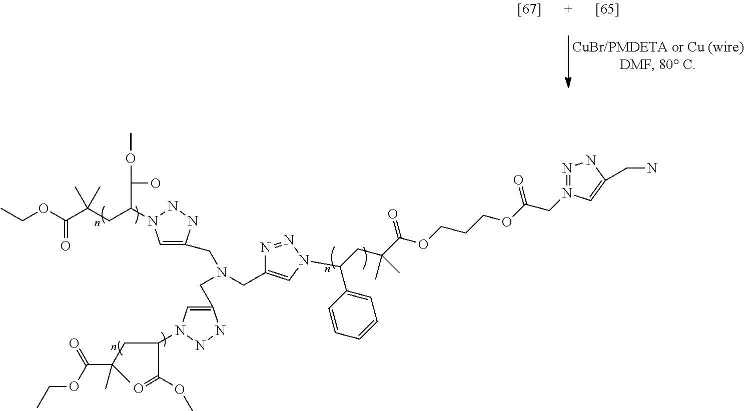

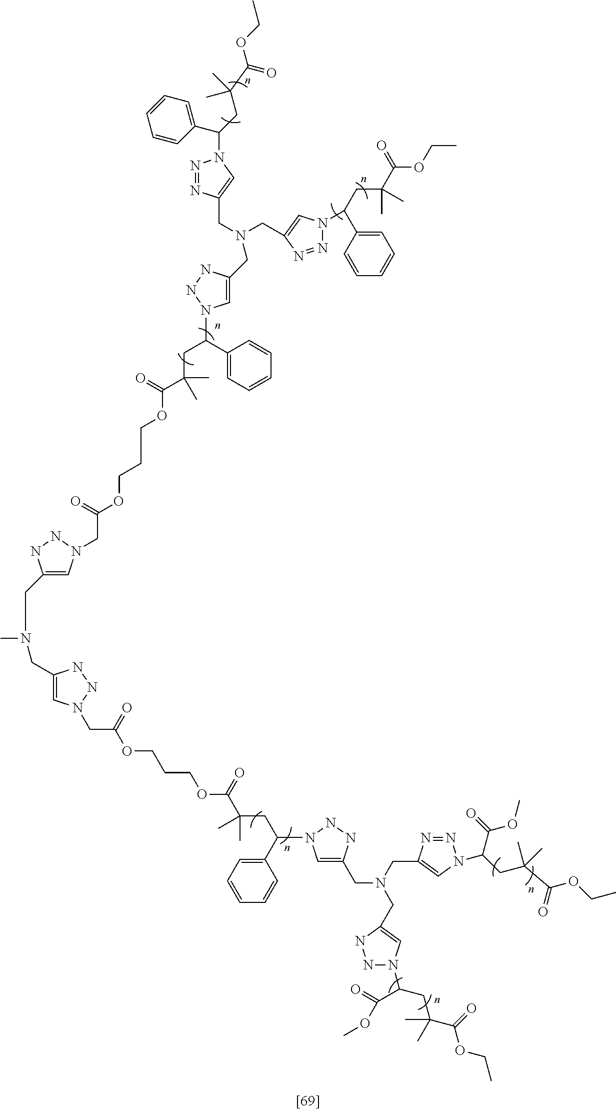

### Synthesis of (PSTY)₂—PSTY—(PSTY—(PMA)₂)₂ [70]

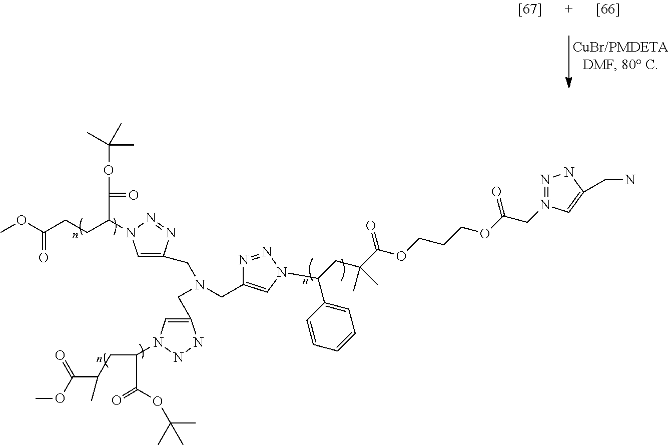

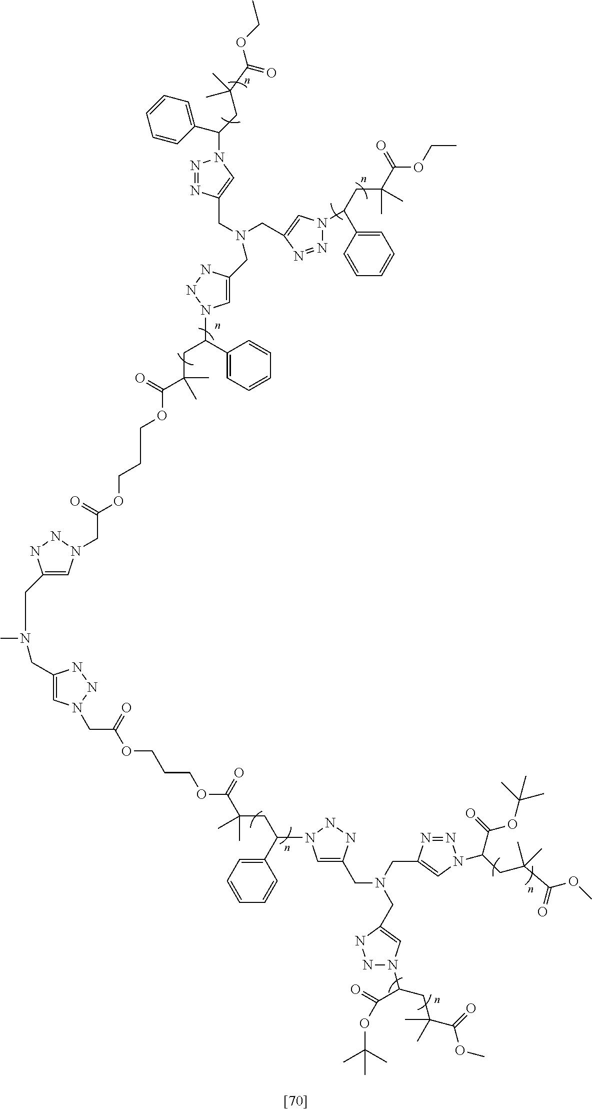

### Degradation of Dendritic Star —(PSTY—(PSTY)₂)₃ [68]

Where required, the mikto-arm star dendrimers of Example 8, in particular [53], can be degraded to obtain the constituent arms as follows:

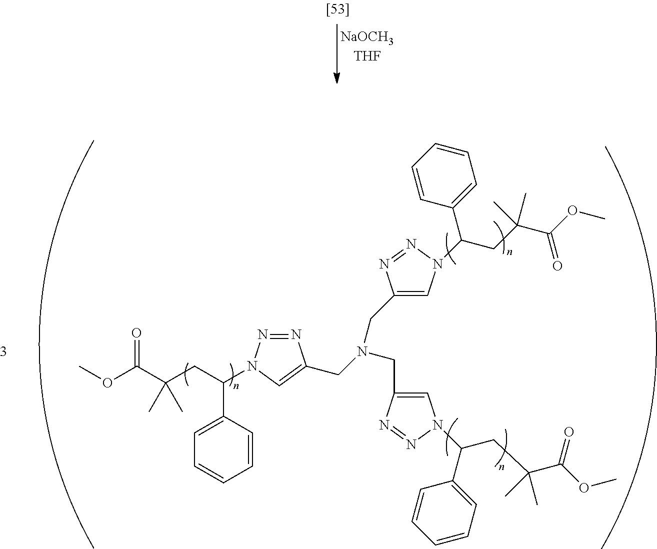

To a 250 μL aliquot of the reaction mixture from the synthesis of —(PSTY—(PSTY)₂)₃ [68] was added THF (1 mL) and NaOCH₃ (10 mg, 1.85×10⁻⁴ mol), The mixture was stirred at room temperature for 16 h, then diluted and analysed by SEC (see FIG. 14),

The number average molecular weight (Mₙ), polydispersity index (PDI) and the yield of the mikto-arm star dendrimers of Example 8 are presented in tabular form overleaf. As is clear from the table, the dendrimers have a narrow polydispersity.

Size Exclusion Chromatography Analyses

Method A: 10×CuBr/PMDETA

| Starting Functional Stars | | Stars/Dendritic Stars | Mₙ | PDI | Yield % |
|---|---|---|---|---|---|
| HO—PSTY—(—≡)₂ [57] | PSTY—N₃ [19] | HO—PSTY—(PSTY)₂ [58] | 16717 | 1.06 | 90 |
| (Mₙ = 6400, PDI = 1.09) | (Mₙ = 5120, PDI = 1.09) | | | | |
| HO—PSTY—(—≡)₂ [57] | P^{t}BA—N₃ [21] | HO—PSTY—(P^{t}BA)₂ [59] | 16315 | 1.08 | 86 |
| (Mₙ = 6400, PDI = 1.09) | (Mₙ = 6180, PDI = 1.10) | | | | |
| HO—PSTY—(—≡)₂ [57] | PMA—N₃ [20] | HO—PSTY—(PMA)₂ [60] | 14224 | 1.05 | 92 |
| (Mₙ = 6400, PDI = 1.09) | (Mₙ = 3900, PDI = 1.08) | | | | |
| (≡—)₂—PSTY—(PSTY)₂ [67] | N₃—PSTY—(PSTY)₂ [64] | —(PSTY—(PSTY)₂)₃ [68] | 45209 | 1.11 | 57 |
| (Mₙ = 16717, PDI = 1.06) | (Mₙ = 16717, PDI = 1.06) | —(PSTY—(PSTY)₂)₃ [68]^{f} | 41153 | 1.11 | — |
| (≡—)₂—PSTY—(PSTY)₂ [67] | N₃—PSTY—(P^{t}BA)₂ [65] | (PSTY)₂—PSTY—(PSTY—(P^{t}BA₂))₂ [69] | 32242 | 1.09 | 55 |
| (Mₙ = 16717, PDI = 1.06) | (Mₙ = 16315, PDI = 1.08) | (PSTY)₂—PSTY—(PSTY—(P^{t}BA₂))₂ [69]^{f} | 31707 | 1.13 | — |
| (≡—)₂—PSTY—(PSTY)₂ [67] | N₃—PSTY—(PMA)₂ [66] | (PSTY)₂—PSTY—(PSTY—(PMA)₂)₂ [70] | 35302 | 1.09 | 41 |
| (Mₙ = 16717, PDI = 1.06) | (Mₙ = 14224, PDI = 1.05) | (PSTY)₂—PSTY—(PSTY—(PMA)₂)₂ [70]^{f} | 42153 | 1.08 | — |

Method B: 0.5×CuBr/PMDETA

| Starting Functional Stars | | Stars/Dendritic Stars | Mₙ | PDI | Yield % |
|---|---|---|---|---|---|
| HO—PSTY—(—≡)₂ [57] | PSTY—N₃ [19] | HO—PSTY—(PSTY)₂ [58] | 14975 | 1.06 | 91 |
| (Mₙ = 6260, PDI = 1.09) | (Mₙ = 5600, PDI = 1.09) | | | | |
| HO—PSTY—(—≡)₂ [57] | P^{t}BA—N₃ [21] | HO—PSTY—(P^{t}BA)₂ [59] | 15000 | 1.08 | 83 |
| (Mₙ = 6260, PDI = 1.09) | (Mₙ = 6180, PDI = 1.10) | | | | |
| HO—PSTY—(—≡)₂ [57] | PMA—N₃ [20] | HO—PSTY—(PMA)₂ [60] | 14894 | 1.08 | 90 |
| (Mₙ = 6260_{,} PDI = 1.09) | (Mₙ = 3900, PDI = 1.08) | | | | |
| (≡—)₂—PSTY—(PSTY)₂ [67] | N₃—PSTY—(PSTY)₂ [64] | —(PSTY—(PSTY)₂)₃ [68] | 41945 | 1.11 | 64 |
| (Mₙ = 14975, PDI = 1.06) | (Mₙ = 14975, PDI = 1.06) | | | | |
| (≡—)₂—PSTY—(PSTY)₂ [67] | N₃—PSTY—(P^{t}BA)₂ [65] | (PSTY)₂—PSTY—(PSTY—(P^{t}BA₂))₂ [69] | — | — | 7 |
| (Mₙ = 14975, PDI = 1.06) | (Mₙ = 15000, PDI = 1.08) | | | | |
| (≡—)₂—PSTY—(PSTY)₂ [67] | N₃—PSTY—(PMA)₂ [66] | (PSTY)₂—PSTY—(PSTY—(PMA)₂)₂ [70] | — | — | 29 |
| (Mₙ = 14975, PDI = 1.06) | (Mₙ = 14894, PDI = 1.08) | | | | |

Method C: Cu (Wire)

| Starting Functional Stars | | Stars/Dendritic Stars | Mₙ | PDI | Yield % |
|---|---|---|---|---|---|
| HO—PSTY—(—≡)₂ [57] | PSTY—N₃ [19] | HO—PSTY—(PSTY)₂ [58] | 15270 | 1.07 | 85 |
| (Mₙ = 5500, PDI = 1.09) | (Mₙ = 5600, PDI = 1.09) | | | | |
| (≡—)₂—PSTY—(PSTY)₂ [67] | N₃—PSTY—(PSTY)₂ [64] | —(PSTY—(PSTY)₂)₃ [68] | 36061 | 1.16 | 76 |
| (Mₙ = 15270, PDI = 1.07) | (Mₙ = 15270, PDI = 1.07) | —(PSTY—(PSTY)₂)₃ [68]^{f} | 36742 | 1.17 | — |
| (prepared by Method B) | | (prepared by Method C) | | | |
| (≡—)₂—PSTY—(PSTY)₂ [67] | N₃—PSTY—(PSTY)₂ [64] | —(PSTY—(PSTY)₂)₃ [68] | 43737 | 1.12 | 66 |
| (Mₙ = 14975, PDI = 1.06) | (Mₙ = 14975, PDI = 1.06) | | | | |
| (≡—)₂—PSTY—(PSTY)₂ [67] | N₃—PSTY—(P^{t}BA)₂ [65] | (PSTY)₂—PSTY—(PSTY—(P^{t}BA₂))₂ [69] | 35598 | 1.10 | 61 |
| (Mₙ = 14975, PDI = 1.06) | (Mₙ = 15000, PDI = 1.08) | | | | |
| (≡—)₂—PSTY—(PSTY)₂ [67] | N₃—PSTY—(PMA)₂ [66] | (PSTY)₂—PSTY—(PSTY—(PMA)₂)₂ [70] | 34163 | 1.15 | 62 |
| (Mₙ = 14975, PDI = 1.06) | (Mₙ = 14894, PDI = 1.08) | | | | |

### Example 9

### Synthesis of Functional Arm Star Polymers

HO—PSTY—Br [15], HO—PSTY—N₃ [24] functional arm HO—PSTY-(—≡)₂ [57] were synthesised as discussed previously.

### Synthesis of Functional Arm Star HO—PSTY—(PSTY)₂ [58]

PSTY—N₃ [19] and P^{t}BA-N₃ [21] were prepared from the corresponding PSTY—Br [10] (Mn=5000, PDI=1.10) and P^{t}BA-Br [12] (Mn=6200, PDI=1.10) as previously discussed. HO—PSTY-(—≡)₂ [57] (0.198 g, 3.60×10⁻⁵ mol), PSTY—N₃ (19, 0.4234 g, 7.56×10⁻⁵ mol), Cu (wire, 1.0 g) and 6 ml, of DMF were added to a 10 mL Schlenk flask equipped with a magnetic stirrer. The reaction mixture was stirred for 4 h at 80° C. in a temperature controlled oil bath. The functional arm star [58] i.e. G₂[G₁ PSTY—OH, G₂PSTY₂] was precipitated into cold methanol, filtered and dried under high vacuum at 25° C.

The above procedure was repeated for the synthesis of the functional mikto-arm, HO—PSTY—P^{t}BA)₂ [59] i.e. G₂[G₁PSTY—OH, G₂P^{t}BA-N₂] using P^{t}BA-N₃ [21]. These polymers were purified by precipitation into water, then filtered and dried under high vacuum at 25° C.

### Synthesis of Functional Arm Star Br—PSTY—(PSTY)2 [61]

58 (0.50 g, 2.94×10⁻⁵ mol), TEA (4.5 μL, 3.2×10⁻⁵ mol) and 1.5 mL of dry DCM were added to a 10 mL Schlenk flask equipped with stirrer bar while purged with N₂. Bromoacetyl bromide [1] (12.7 μL, 1.47×10⁻⁴ mol) in 0.5 mL of dry DCM was added dropwise under N₂ to the stirred mixture over a period of 20 min. at room temperature. After complete addition the mixture was allowed to stir for a further 16 h at room temperature. The polymer was precipitated in MeOH, then filtered and washed 3 times with MeOH (20 mL). The recovered polymer G₂[G₁PSTY—Br, G₂PSTY₂] i.e. [61] was dried under high vacuum at 25° C.

The above procedure was used for the synthesis of the functional mikto-arm stars G₂[G₁PSTY—Br, G₂P^{t}BA₂] or [62] using G₂[G₁PSTY—OH, G₂P^{t}BA₂] i.e. [59].

### Synthesis of Functional Arm Star N3-PSTY—(PSTY)₂ 64]

NaN₃ (0.0166 g, 2.67×10⁻⁴ mol) was added to a stirred solution of functional arm star G₂[G₁PSTY—Br, G₂PSTY₂] [61] (0.450 g, 2.67×10⁻⁵ mol) in 2 mL DMF. The reaction mixture was stirred for 24 h at 50° C. in a temperature controlled oil bath. The polymer G₂[G₁PSTY—N₃, G₂PSTY₂] [64] was precipitated in water with vigorous stirring, filtered and dried under high vacuum at 25° C.

The above procedure was used for the synthesis of the functional mikto-arm stars G₂[G₁PSTY—N₃, G₂P^{t}BA₂] 65, using G₂[G₁PSTY—Br, G₂P^{t}BA₂] 62.

### Example 10

### Synthesis of Click Multifunctional Star Cores

The 4-arm star multi-functional initiator pentaerythritol tetrakis(2-bromopropionate) (4BrPr) and 3-hydroxypropyl 2-bromo-2-methylpropanoate were synthesized according to published procedures (Matyjaszewski, K.; Miller, P. J.; Pyun, J.; Kickelbick, G.; Diamanti, S. Macromolecules 1999, 32, 6526-6535).

Star P(tBA-Br)₄: [^{t}BA]: [4BrPr]:[Cu(1)]:[Cu(2)]:[PMDETA]=[250]:[1]:[4]:[0.4]:[4.4] [71]

Freshly purified ^{t}BA (2.20 g, 1.72×10⁻² mol), PMDETA (0.0525 g, 3.50×10⁻⁴ mol), pre-formed CuBr₂ (0.0062 g, 2.76×10⁻⁵ mol), and pentaerythritol tetrakis(2-bromopropionate) (4BrPr, 0.0465 g, 6.89×10⁻⁵ mol) were added to a 10 mL Schlenk flask equipped with a magnetic stirrer and purged with N₂ for 15 min. CuBr (0.0395 g, 2.76×10⁻⁴ mol) was then carefully added under positive N₂ flow and then purged with N₂ for a further 10 min. The flask was placed in a temperature controlled oil bath at 35° C. for 2 h. The reaction was terminated by quenching in liquid nitrogen and then exposure to air. The polymerization mixture was diluted with THF then the copper salts removed by passage through an activated basic alumina column. The solution was concentrated by airflow and the polymer recovered by precipitation into methanol/water (50:50 vol), filtered and dried for 48 h under high vacuum at 25° C. The polymer was characterized by LS-SEC. (Mₙ=19000 and PDI=1.09) to yield Star P(^{t}BA₃₇-Br)₄ [71a]

In a similar manner Star P(^{t}BA₁₁₇-Br)₄ [71b], was synthesized (Mn=60 000 and PDI=1.11) with [^{t}BA]:[4BrPr]:[Cu(1)]:[Cu(2)]:[PMDETA]=[2800]:[1]:[4]:[0.4]:[4.4].

### Synthesis of Star P(^{t}BA₃₇-N₃)₄ [72]

NaN₃ (0133 g, 2.1×10⁻³ mol) was added to a stirred solution of [71a] (1.00 g, 5.26×10⁻³ mol) in 5 mL DMF. The reaction mixture was stirred for 24 h at 50° C. in a temperature controlled oil bath. into methanol/water (50:50 vol), then filtered and dried under high vacuum at 25° C. to obtain 172a].

In a similar manner Star P(^{t}BA₁₁₇-N₃)₄ [72b] was synthesized from [71b]

### Synthesis of Star P(tBA₃₇-(≡)₂)₄ [73]

Star P(tBA₃₇-N₃)₄ (72a, 0.500 g, 2.63×10⁻⁵ mol), PMDETA (0.183 g, 1.05×10⁻³ mol), TPA (0.275 g, 2.1×10⁻³ mol), CuBr (0.150 g, 1.05×10⁻³ mol)and 5 mL of DMF was added to a 10 mL Schlenk flask equipped with a magnetic stirrer. The reaction mixture was stirred for 4 h at 80° C. in a temperature controlled oil bath. The polymer was recovered by precipitation into an acidified MeOH/water (50:50 vol) mixture and then filtered. The polymer was redissolved in DMF (6 mL) and re-precipitated into an acidified MeOH/water (50:50 vol) mixture, recovered by filtration and washed exhaustively with water. The polymer [73a] was dried under high vacuum at 25° C.

In a similar manner Star P(^{t}BA₁₁₇-(≡)₂)₄ [73b] was synthesized from [72b].

### Synthesis of G₃[G₁P(^{t}BA₃₇)₄, G₂PSTY₈, G₃PSTY₁₆] [74]

Star P(^{t}BA₃₇-(≡)₂)₄ ([73], 0.005 g, 2.63×10⁻⁷ mol), PMDETA (0.0037 g, 2.11×10⁻⁵ mol), G₂[G₁PSTY—N₃, G₂PSTY₂] ([64], 0.0394 g, 2.32×10⁻⁶ mol) and 1 mL of DMF was added to a 10 mL Schlenk flask equipped with a magnetic stirrer. The solution was degassed by bubbling with N2 gas for 15 min then CuBr (0.003 g, 2.11×10⁻⁵ mol) was added under a nitrogen blanket. The reaction mixture was stirred for 4 h at 80° C. in a temperature controlled oil bath. The solution was taken to dryness under an air stream and taken up into 1 mL of THF. A sample was removed for GPC analysis and after the product [74a] was identified it was recovered from the mixture by preparative GPC.

In a similar manner G₃[G₁P(^{t}BA₁₁₇)₄, G₂PSTY₈, G₃PSTY₁₆] [74b], G₃[G₁P(^{t}BA₃₇)₄, G₂PSTY₈, G₃PS^{t}BA₁₆] [75], G₃[G₁P(^{t}BA₄, G₂PSTY₈, G₃PS^{t}BA₁₆] [76] were prepared.

Examples 9 and 10 will now be discussed in further detail.

Two 4-arm P^{t}BA stars [71a] and [71b] of different molecular weight were prepared using CuBr, a CuBr2/PMDETA complex and pentaerythritol(2-bromopropionate) at 35° C. in bulk. Polymer [71a] reached 60% conversion after 2 h with a number-average molecular weight (Mₙ) of 19000 and polydispersity index (PDI) of 1.09. The second polymer [71b] also gave ideal ‘living’ behavior (Mₙ=60000 and PDI=1.11) after 2 h, reaching a conversion of 18%. The Br end-groups on the stars were then converted to azide by reacting [71a] or [71b] with NaN₃ in DMF for 24 h at 50° C. to form [72a or 72b], respectively, and further converted to dialkynes through a ‘click’ reaction of [72a or 72b] with tripropagyl amine [3] in DMP for 4 h at 80° C. to form [73a or 73b] (see Scheme 1).

Scheme 2 shows the methodology to make the reactive 2^{nd} generation polystyrene dendrons. The initial starting PSTY telechelic chain, [15] (Mₙ=6258 and PDI=1.10), was made by ATRP using an initiator with an alcohol and the Br chain-end converted to an azide [24]. Tripropagyl amine [3] was then coupled onto [24] to give the reactive dipropagyl [57]. Another PSTY—Br (Mₙ=5000, PDI=1.10) [10] was made by ATRP and the Br group converted to an azide [19]. This polymer was then coupled to [57] to form a 3-arm star [58] (Mₙ=14000, PDI=1.10), in which the OH group was converted in a two step process to an azide, [64] as described earlier. However, copper wire was used in the absence of added ligand when a triazol ring in the polymer structure was present. The Mₙ for [58] is close to the expected value for attaching two PSTY chains onto [57], supporting the formation of 3-arm stars. The low PDI value suggests we have made [64] in high yields and high purity. Importantly, the use of copper wire resulted in excellent coupling, and provided a constant source of copper. The main advantage is that copper can be separated from the dendron by simply removing the copper wire from the reaction mixture.

Coupling the 4-arm star (73a or 73b) to 64 produced a 3^{rd} generation dendrimer (74a and 74b in Scheme 3) where the 1^{st} generation consists of P^{t}BA and the 2^{nd} and 3^{rd} generational layers consist of PSTY. The ‘click’ reaction was carried out at for 4 h at 80° C. in the presence of PMDETA and CuBr. The purity of the dendrimers were high and close to 80%. The dendrimers were further purified through fractionation through SEC to obtain the dendrimers [74a] and [74b] in pure form. The SEC data for 11 and 12 after fractionation gave Mₙ's of 145000 and 195000, respectively, which are close to the calculated values.

This together with the low PDIs of 1.14 and 1.10 show that pure 3^{rd} generation dendrimers can be made from linking polymeric building blocks in a convergent method.

The number average hydrodynamic diameter, Dₕ, was determined to be 23 nm by Dynamic Laser Scattering (DLS).

The examples demonstrate the synthesis of high order polymer architectures (3^{rd} generation dendrimers) by coupling reactive dendrons onto a 4-arm star (made by ATRP). This is a unique method to make such architectures and open the way for a wide range of architectural control.

### Example 11

### Polymer Micelle Formation

In structures according to the invention, it is possible to convert the acrylate polymers to acid groups by deprotection as follows.

Deprotection of the ^{t}BA blocks to yield acrylic acid blocks was carried out using published literature procedures (Whittaker, Michael R.; Urbani, Carl N.; Monteiro, Michael J. JACS. 2006, 128(35), 11360-11361).

to give G₃[G₁P(AA₃₇)₄, G₂PSTY₈, G₃PSTY₁₆] [77a], G₃[G₁P(AA₁₁₇)₄, G₂PSTY₈, G₃PSTY₁₆] [77b], G₃[G₁P(AA₃₇)₄, G₂PSTY₈, G₃PAA₁₆] [78] and

G₃[G₁P(AA₁₁₇)₄, G₂PSTY₈, G₃PAA₁₆] [79] from 174a], [74b], [75] and [76] respectively.

Amphiphilic polymer micelles were obtained by the gradual addition (0.025 mL/min) of nonsolvent (Millipore H₂O, total volume 2.4 mls) for the hydrophobic poly(PSTY) blocks to 0.1 ml of the 10 mg/mL polymer DMF solutions prepared from either the amphiphilic block polymers with gentle stirring. The final total volume was 2.5 mL of aqueous micelle solution giving final concentration of 0.4 mg/mL of polymer. The micelles were exhaustively dialysed against Millipore water ph=6.8 using presoaked and rinsed dialysis bags (Pierce Snakeskin, MWCO 3K). The polymer micelles were characterised by dynamic light scattering (DLS) and transmission electron microscopy (TEM).

### Example 12

### Coupling of siRNA onto Dendrimer

This example demonstrates that it is possible to couple an active molecule to a dendrimer according to the invention.

Capping of OH Groups with Succinic Anhydride (SA)

Sym-G₀-G₁-G₂-P^{t}BA-(OH)₂ [55] (44 mg, 6.77×10⁻⁷ moles, 1.08×10⁻⁵ moles OH groups) was dissolved into 1 mL of anhydrous DMF. To this solution was added succinic anhydride (10.8 mg, 1.08×10⁻⁴ moles, 10 equiv. to OH groups) and the solution stirred at room temperature for 3 days. The solution was quenched with the addition of 0.2 mL Millipore water and stirred for a further 3 days at room temperature. The solution was diluted with 50 mL chloroform and the organic phase extracted with water (*2, 100 mL) and brine (*1, 100 mL). The organic phase was dried with anhydrous magnesium sulfate, filtered and the product recovered by rotary evaporation. The capped Sym-G₀-G₁-PSTY-G₂-P^{t}BA-(COOH)₂ [55a] was then exhaustively dried at room temperature under high vacuum for 48 hr.

Deprotection of tBA Units with TFA to Form Acrylic Acid (AA) Units

The dendrimer Sym-G₀-G₁-PSTY-G₂-P^{t}BA-(COOH)₂ [55a] above (22 mg, 3.39×10⁻⁷ moles, 8.66×10⁻⁵ moles ‘BA) was dissolved into 0.45 mL dry DCM. To this solution was added trifluoroacetic acid (TFA) (40 mg, 4.33×10⁴ moles, 5 equiv. to tert-butyl acrylate units) and the solution stirred overnight at room temperature. The reaction mixture was taken to dryness with a nitrogen stream then exhaustively dried at room temperature under high vacuum for 48 hr to give amphiphilic dendrimer Sym-G₀-G₁-PSTY-G₂-PAA-(COOH)₂ [55b]

Coupling siRNA to 155b]

Note: siRNA Duplex

Sense_S:
5′-(amine)rGrCrArCrGrArCUUrCUUrCrArArGUrCrC UU
Sense_A:
5′-rGrCrArCUUrGrArArGrArArGUrCrGUrCrC UU

A stock solution of the amphiphilic capped dendrimer [55b] was prepared by taking it up into 3.31 mL of anhydrous dimethyl formamide (DMF) to give a final concentration of 1.02×10⁻⁷ moles/mL DMF. To 0.5 mL of this solution (which contains 5.12×10⁻⁸ moles [55b] or 1.31×10⁻⁵ moles of total acrylic acid groups present in dendrimer) was added EDC (12.5 mg, 6.55×10⁻⁵ moles, 5 equiv. to total acrylic acid groups) and the solution stirred for 30 min under nitrogen.

To the above solution was added NH₂-siRNA duplex (2.56×10⁻⁸ moles, 0.5 eqiv. to dendrimer 155b]) dispersed into 0.5 mL DMF. The solution was stirred for 1 hr after which 1 ml of freshly glass distilled RNAse free water was added to improve solubility of the siRNA. The solution was stirred under nitrogen for a further 2 days. The reaction solution was then diluted with a further 5 mL of freshly glass distilled RNAse free water and dialyzed against freshly glass distilled RNAse free water for 3 days using snakeskin dialysis tubing (10 k mol. Wt. cut off). The dendrimer-siRNA conjugate Sym-G₀-G₁-G₂-PAA-(COOH)₂-siRNA [55c] was recovered by freeze drying.

Micellisation of the Dendrimer-siRNA Conjugate [55c]

A stock micellisation solution was prepared by taking the dendrimer-siRNA conjugate [55c] into DMF (2.9 mg in 0.570 mL of DMF) resulting in a 0.5% w/w solution. To 0.1 mL of this solution was added 1 mL of freshly glass distilled RNAse free water drop-wise at 0.013 mL/min while stirring. After the complete addition of water the micelle solution was dialysed against freshly glass distilled RNAse free water for 2 days using snakeskin dialysis tubing (10 k mol. wt. cut off). Number average hydrodynamic diameter, Dₕ, was determined to be 85 nm by DLS. [siRNA]=4459 nM. It should be noted that Dₕ was approximately 18 nm of amphiphilic dendrimer Sym-G₀-G₁-G₂-PAA-(COOH)₂ [55b].

Micellisation of the Dendrimer [55b], Control Experiment

To a solution of [55b] in DMF (1 mg in 0.2 mL DMF 0.5% w/w solution) was added 2 mL of freshly glass distilled RNAse free water drop-wise at 0.013 mL/min while stirring. After the complete addition of water the micelle solution (was dialysed against freshly glass distilled RNAse free water for 2 days using snakeskin dialysis tubing (10 k mol. wt. cut off).

Example 12 is represented in Scheme 4.

1-37. (canceled)

38. A dendron including:
a first polymer;
two or more first generation polymers bound to the first polymer; and
wherein the first generation polymers include a functional group having at least one active site capable of bonding to a complementary functional group having at least one active site of a predetermined number of further generation polymers, and wherein the first polymer includes a functional group having at least one active site capable of bonding to a complementary functional group having at least one active site of another dendron.

39. A dendron according to claim 38 wherein the first polymer includes a functional group having at least one active site capable of bonding to a complementary functional group having at least one active site of another dendron either directly or indirectly through a linking group and wherein the linking group is formed by a compound reacted with the dendrons simultaneously or sequentially.

40. A dendron according to claim 38 wherein the terminal generation polymer(s) comprise a functional group having an active site capable of bonding to one or more polymers thereby to form a further generation and wherein the functional group is a single functional group or multiple functional groups.

41. A dendron according to claim 38 wherein the functional group is terminal or located along the length of the generation polymer.

42. A dendron according to claim 38 wherein the functional group has one or more active sites.

43. A dendritic molecule comprising two or more dendrons wherein each of the dendrons is according to claim 38.

44. A dendritic molecule comprising two or more dendrons bound together by a common multifunctional group, wherein each of the dendrons is according to claim 38.

45. A dendritic molecule comprising at least three dendrons wherein each of the dendrons is according to claim 38.

46. A dendritic molecule according to claim 43 wherein at least two of the dendrons are different.

47. A dendritic molecule according to claim 46 wherein at least one of the dendrons comprises a first polymer having a functional group that has two or more active sites.

48. A dendritic molecule according to claim 47 wherein the at least one dendron is coupled to two or more dendrons, each of these dendrons comprising a first polymer having a functional group with one active site.

49. A dendritic molecule according to claim 43 wherein the dendrons are represented as G₂[G₁Pₐ—X, G₂P_{b}] where G₁, and G₂ are a first generation and a second generation, Pₐ is the first generation polymer comprising X, a functional group having an active site at its proximal end and P_{b} is the second generation polymer.

50. A dendritic molecule according to claim 49 wherein Pₐ and P_{b} are the same or different.

51. A dendritic molecule according to claim 50 that is a mikto-arm dendrimer wherein P_{b} of a first dendron is different from P_{b} of a second dendron.

52. A dendritic molecule comprising:
a core or first polymer that is a star polymer comprising three or more arms, at least one arm comprising a functional group having an active site; and
one or more generation polymers or one or more dendrons bound to the active site.

53. A dendritic molecule according to claim 52 wherein the star polymer has one or more first generation polymers bonded to each of the arms and each generation polymer is bonded to a predetermined number of further generation polymers extending outwardly from the first generation polymer.

54. A dendritic molecule according to claim 52 wherein the star polymer has one or more dendrons bonded to each of the arms.

55. A dendritic molecule according to claim 52 wherein the star polymer is prepared from a multifunctional initiator.

56. A dendritic molecule according to claim 52 which is a third generation dendrimer represented as G₃[G₁Pₐ, G₂P_{b}, G₃P_{c}] wherein G₁, G₂ and G₃ represent a first, second and third generation respectively and Pₐ, P_{b} and P_{c} are a first, second and third generation polymer respectively that may be the same or different.

57. A dendritic molecule according to claim 43 wherein the dendrons are symmetric.

58. A dendritic molecule according to claim 43 wherein the dendrons are asymmetric.

59. A dendron according to claim 38 that has degradable linkages.

60. A dendritic molecule according to claim 43 that has degradable linkages.

61. A dendron according to claim 38 which is an amphiphilic molecule.

62. A dendritic molecule according to claim 43 that is an amphiphilic molecule.

63. A dendron according to claim 38 which is functionalised by the bonding of one or more chemical moieties to the outermost polymers of the dendron or dendritic molecule, the bonding of one or more chemical moieties to the intermediate polymers, and/or encapsulating one or more small molecules within the cavities within the dendron or dendritic molecule.

64. A method of forming a dendron comprising the steps of:
(a) forming a first polymer comprising a functional group having at least one active site;
(b) bonding at least one first generation polymer to the at least one active site of the first polymer to form a first generation macromolecule, said first generation polymer comprising at least one functional group having at least one active site;
(c) bonding at least one further generation polymer to the at least one active site of the first generation polymer to form a second generation macromolecule; and
(d) wherein said further generation polymer includes at least one functional group having at least one active site capable of bonding to at least a further generation polymer.

65. A method of forming a dendron according to claim 38 for the formation of a dendritic molecule comprising the steps of:
(a) forming a first polymer;
(b) bonding a functional group having at least one active site to the first polymer;
(c) bonding at least one generation polymer to the at least one active site of the first polymer to form a first generation macromolecule;
(d) bonding a functional group having at least one active site to at least one generation polymer of the macromolecule to provide at least one active site on the macromolecule; and
(e) bonding at least one further generation polymer to the at least one active site on the macromolecule; and
(f) repeating steps (d) and (e) until a predetermined number of generation polymers have been added.

66. A method of forming a dendritic molecule, wherein the dendritic molecule comprises two or more dendrons prepared by the method of claim 64.

67. A method of forming a dendritic molecule comprising the step of coupling two or more dendrons according to claim 38.

68. A method of convergently forming a dendritic molecule comprising the steps of:
(a) forming a plurality of dendrons, each dendron being formed by the steps of
(1) forming a first polymer,
(2) bonding a functional group having at least one active site to the polymer,
(3) bonding at least one generation polymer to the at least one active of the polymer to form a first generation macromolecule,
(4) bonding a functional group having at least one active site to the at least one generation polymer end of the macromolecule,
(5) bonding at least one further generation polymer to the at least one active site of the macromolecule to provide an active site on the macromolecule, and
(6) repeating steps (4) and (5) until a predetermined number of generation polymers have been added, and
(b) bonding a multifunctional group having two or more active sites to the non-functionalised end of the first polymer and bonding two or more dendrons to the active sites of the multifunctional group bonded to the first polymer.

69. A method of forming a dendritic molecule according to claim 68 wherein a first dendron comprising a functional group having two or more active sites bonded to a non-functionalised end of the first polymer is coupled to two or more dendrons.

70. A method of forming a dendritic molecule according to claim 69 wherein the dendrons are represented as G₂[G₁Pₐ—X, G₂P_{b}].

71. A method of forming a dendritic molecule comprising the steps of:
forming a first polymer comprising two or more functional groups having at least one active site;
bonding two or more first generation polymers with the active sites to form a first generation macromolecule thereby forming a first generation macromolecule wherein the first generation polymer comprises two or more functional groups having at least one active site; and
iteratively bonding further generation polymers to the active site on the first generation macromolecule, each iterative step resulting in a generation macromolecule having a functional group with an active site until termination.

72. A method of divergently forming a dendritic molecule comprising the steps of:
(a) forming a first polymer
(b) bonding two or more functional groups having at least one active site to the first polymer;
(c) bonding two or more generation polymers to the active sites on the first polymer to form a first generation macromolecule;
(d) bonding one or more functional groups having at least one active site to a plurality of sites on the first generation macromolecule;
(e) repeating steps (c) and (d) until a predetermined number of generation polymers have been added.

73. A method of forming a dendritic molecule according to claim 71 wherein the two functional groups are at terminal ends of the first polymer.

74. A method of forming a dendritic molecule according to claim 71 wherein the first polymer is a star polymer, each of whose arms has one or more functional groups having an active site.

75. A method of forming a dendritic molecule comprising the steps of forming a star polymer each of whose arms comprises a functional group having an active site and bonding one or more dendrons to the active site.

76. A dendritic molecule obtained by the method of claim 75 that is symmetrical.
